(19) United States
(12) Patent Application Publication
(10) Pub. No.: US 2023/0273519 A1
(43) Pub. Date: Aug. 31, 2023

(54) RADIATION-SENSITIVE RESIN COMPOSITION AND PATTERN FORMATION METHOD

(71) Applicant: JSR CORPORATION, Tokyo (JP)

(72) Inventor: Ken MARUYAMA, Tokyo (JP)

(73) Assignee: JSR CORPORATION, Tokyo (JP)

(21) Appl. No.: 18/024,309

(22) PCT Filed: Aug. 17, 2021
(86) PCT No.: PCT/JP2021/029984

§ 371 Date: Mar. 2, 2023

(30) Foreign Application Priority Data
Nov. 12, 2020 (JP) 2020-188515
Feb. 12, 2021 (JP) 2021-020781

(51) Int. Cl. G03F 7/038; C08F 220/28; G03F 7/039

(52) U.S. Cl. CPC G03F 7/038; C08F 220/28; G03F 7/039

(57) ABSTRACT

Provided is a radiation-sensitive resin composition capable of exhibiting sensitivity and CDU performance at a sufficient level when a next-generation technology is applied, and a pattern formation method. A radiation-sensitive resin composition containing: a radiation-sensitive acid generating resin comprising a repeating unit A having an acid-dissociable group represented by the following formula (1) and a repeating unit B including an organic acid anion moiety and a sulfonium cation moiety containing an aromatic ring structure having a fluorine atom; and a solvent; in the formula (1), R^{T} is a hydrogen atom, a fluorine atom, a methyl group, or a trifluoromethyl group; R^{X} is a monovalent hydrocarbon group having 2 to 20 carbon atoms; and Cy represents an alicyclic structure having 3 to 20 ring members and formed together with a carbon atom to which this is bonded.

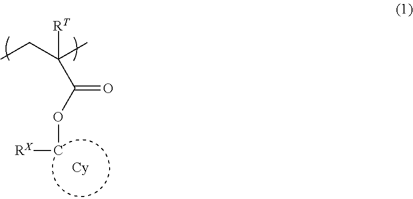

### TECHNICAL FIELD

The present invention relates to a radiation-sensitive resin composition and a pattern formation method.

### BACKGROUND ART

A photolithography technology using a resist composition has been used for the formation of a fine circuit in a semiconductor device. As a representative procedure, for example, a resist pattern is formed on a substrate by generating an acid by irradiating a coating film of the resist composition with radiation through a mask pattern, and then reacting in the presence of the acid as a catalyst to generate a difference in the solubility of a resin into an alkaline or organic solvent-based developer between an exposed area and an unexposed area.

In the photolithography technology, pattern miniaturization is promoted by using short-wavelength radiation, such as ArF excimer laser or by combining such radiation with an immersion exposure method (liquid immersion lithography). As a next-generation technology, further short-wavelength radiation, such as an electron beam, an X-ray, and an extreme ultraviolet ray (EUV) is being utilized, and a resist material containing an acid generator with a benzene ring having enhanced radiation absorption efficiency is also being studied. (Patent Document 1)

### PRIOR ART DOCUMENTS

### Patent Document

Patent Document 1: JP-A-2014-2359

### SUMMARY OF THE INVENTION

### Problems to be Solved by the Invention

Even in the above-described next generation technology, various resist performances equivalent to or higher than conventional performances are required in sensitivity and critical dimension uniformity (CDU) performance, which is an index of uniformity of a line width and a hole diameter, and the like.

An object of the present invention is to provide a radiation-sensitive resin composition capable of exhibiting sensitivity and CDU performance at a sufficient level when a next-generation technology is applied, and a pattern formation method.

### Means for Solving the Problems

As a result of intensive studies to solve the present problems, the present inventors have found that the above object can be achieved by adopting the following configurations, and have accomplished the present invention.

The present invention relates to, in one embodiment,
a radiation-sensitive resin composition comprising:
a radiation-sensitive acid generating resin containing a repeating unit A having an acid-dissociable group represented by the following formula (1) and a repeating unit B comprising an organic acid anion moiety and a sulfonium cation moiety containing an aromatic ring structure having a fluorine atom; and
a solvent,

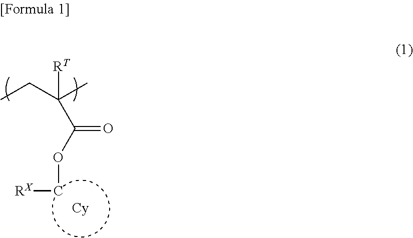

(in the formula (1),
R^{T} is a hydrogen atom, a fluorine atom, a methyl group, or a trifluoromethyl group;
R^{X} is a monovalent hydrocarbon group having 2 to 20 carbon atoms; and
Cy represents an alicyclic structure having 3 to 20 ring members and formed together with a carbon atom to which this is bonded.)

In another embodiment, the present invention relates to:
a radiation-sensitive resin composition comprising:
a radiation-sensitive acid generating resin containing a repeating unit A having an acid-dissociable group represented by the following formula (1) and a repeating unit C having an organic acid anion moiety and an onium cation moiety,
an onium salt containing an organic acid anion moiety and a sulfonium cation containing an aromatic ring structure having a fluorine atom, and
a solvent.

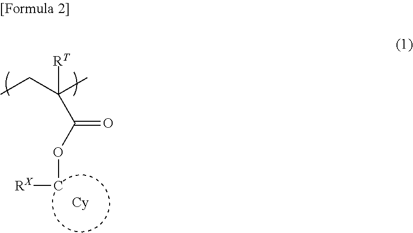

(in the formula (1),
R^{T} is a hydrogen atom, a fluorine atom, a methyl group, or a trifluoromethyl group;
R^{X} is a monovalent hydrocarbon group having 2 to 20 carbon atoms; and
Cy represents an alicyclic structure having 3 to 20 ring members and formed together with a carbon atom to which this is bonded.)

With the radiation-sensitive resin composition, a resist film satisfying sensitivity and CDU performance can be constructed. The reason for this is not clear, but can be expected as follows. Absorption of radiation such as EUV having a wavelength of 13.5 nm by fluorine atoms is very large, and this makes the radiation-sensitive resin composition highly sensitive. In addition, since the acid-dissociable group of the structural unit A in the resin has high acid-dissociation efficiency by exposure, the contrast between an exposed area and an unexposed area is increased, and superior pattern-forming performance is exhibited. It is presumed that the resist performances can be exerted due to these combined actions.

In another embodiment, the present invention relates to a method for forming a pattern, the method comprising:
a step of directly or indirectly applying the radiation-sensitive resin composition to a substrate to form a resist film,
a step of exposing the resist film to light, and
a step of developing the exposed resist film with a developer.

Since the above-described radiation-sensitive resin composition superior in sensitivity and CDU performance is used in the method for forming a pattern, a high-quality resist pattern can efficiently be formed by the method.

### MODE FOR CARRYING OUT THE INVENTION

Hereinbelow, embodiments of the present invention will specifically be described, but the present invention is not limited to these embodiments.

### <Radiation-Sensitive Resin Composition>

A radiation-sensitive resin composition (hereinafter also simply referred to as “composition”) according to the present embodiment comprises a radiation-sensitive acid generating resin and a solvent. The composition may further comprise other optional component as long as the effects of the present invention are not impaired. When the radiation-sensitive resin composition contains the prescribed radiation-sensitive acid generating resin, the radiation-sensitive resin composition can impart high levels of sensitivity and CDU performance to a resulting resist film.

### <Radiation-Sensitive Acid Generating Resin>

The radiation-sensitive acid generating resin (hereinafter also simply referred to as “resin”) is an aggregate (G1) of a polymer containing a repeating unit A having an acid-dissociable group represented by the following formula (1) and a repeating unit B containing an organic acid anion moiety and a sulfonium cation moiety containing an aromatic ring structure having a fluorine atom, or an aggregate (G2) of a polymer containing a repeating unit A having an acid-dissociable group represented by the following formula (1) and a repeating unit C having an organic acid anion moiety and an onium cation moiety, or an aggregate comprising both the aggregate (G1) and the aggregate (G2) (hereinafter, the polymer (G1) and the polymer (G2) are each also referred to as “base resin”). The base resin may contain, in addition to the structural units A, B, and C, a structural unit D having a phenolic hydroxy group, a structural unit E containing a lactone structure, or the like. Hereinbelow, each of the structural units will be described.

### (Structural Unit A)

The structural unit A (hereinafter also referred to as “repeating unit A”) is represented by the following formula (1).

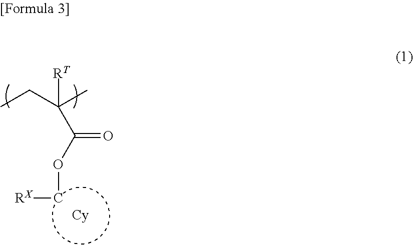

(in the formula (1),
R^{T} is a hydrogen atom, a fluorine atom, a methyl group, or a trifluoromethyl group;
R^{X} is a monovalent hydrocarbon group having 2 to 20 carbon atoms; and
Cy represents an alicyclic structure having 3 to 20 ring members and formed together with a carbon atom to which this is bonded.)

Examples of the monovalent hydrocarbon group having 2 to 20 carbon atoms represented by the R^{X} include chain hydrocarbon groups having 2 to 10 carbon atoms, monovalent alicyclic hydrocarbon groups having 3 to 20 carbon atoms, and monovalent aromatic hydrocarbon groups having 6 to 20 carbon atoms.

Examples of the chain hydrocarbon group having 2 to 10 carbon atoms include linear or branched saturated hydrocarbon groups having 2 to 10 carbon atoms and linear or branched unsaturated hydrocarbon groups having 2 to 10 carbon atoms.

Examples of the alicyclic hydrocarbon group having 3 to 20 carbon atoms include a monocyclic or polycyclic saturated hydrocarbon group and a monocyclic or polycyclic unsaturated hydrocarbon group. Preferred examples of the monocyclic saturated hydrocarbon groups include a cyclopentyl group, a cyclohexyl group, a cycloheptyl group, and a cyclooctyl group. Preferred examples of the polycyclic cycloalkyl groups include bridged alicyclic hydrocarbon groups such as a norbornyl group, an adamantyl group, a tricyclodecyl group, and a tetracyclododecyl group. The bridged alicyclic hydrocarbon group refers to a polycyclic alicyclic hydrocarbon group in which two carbon atoms that constitute an alicyclic ring and not adjacent to each other are bonded by a bonding chain containing one or more carbon atoms.

Examples of the monovalent aromatic hydrocarbon group having 6 to 20 carbon atoms include aryl groups, such as a phenyl group, a tolyl group, a xylyl group, a naphthyl group, and an anthryl group; and aralkyl groups, such as a benzyl group, a phenethyl group, and a naphthylmethyl group.

As the R^{X}, linear or branched saturated hydrocarbon groups having 2 to 5 carbon atoms and alicyclic hydrocarbon groups having 3 to 12 carbon atoms are preferable.

The alicyclic structure having 3 to 20 ring atoms in Cy is not particularly limited as long as it has an alicyclic structure, and may have a monocyclic, bicyclic, tricyclic, tetracyclic or more polycyclic structure, and may be any of a bridged ring structure, a spiro ring structure, a ring assembly structure in which a plurality of rings are directly bonded by a single bond or a double bond, or a combination thereof. In particular, it preferably has a monocyclic, bicyclic, tricyclic, or tetracyclic bridged ring structure, and it is more preferable to be a ring structure of cyclopentane, cyclohexane, norbornane, adamantane, tricyclo[5.2.1.0^{2,6}]decane, tetracyclo[4.4.0.1^{2,5}.1^{7,10}]dodecane, perhydronaphthalene, or perhydroanthracene, or a derivative thereof.

The structural unit A is preferably represented by the following formulas (A-1) to (A-8), for example.

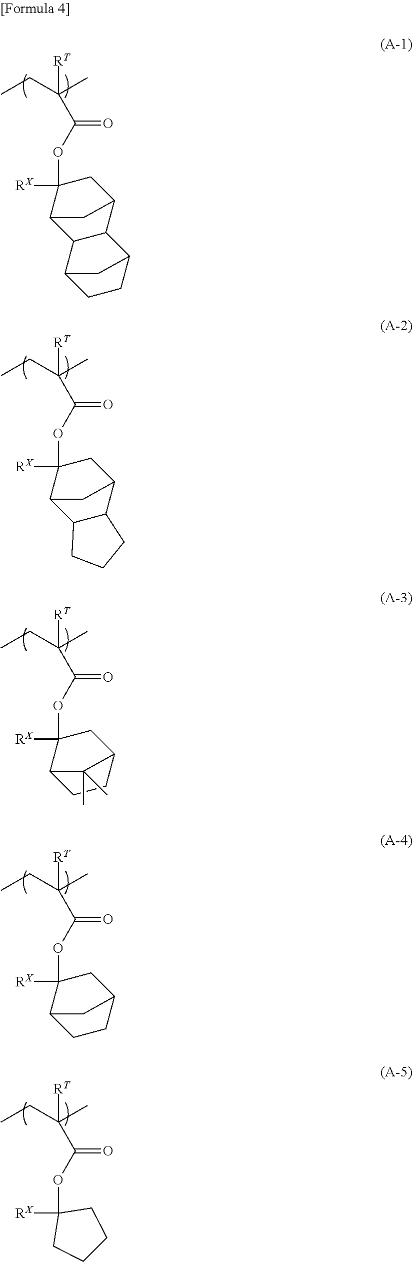

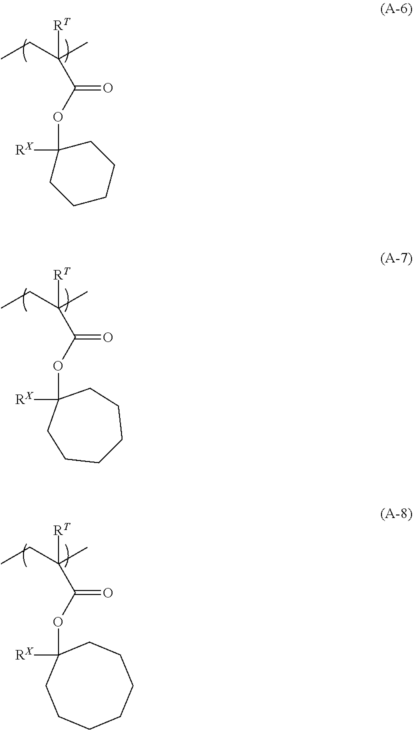

In the formulas (A-1) to (A-8), R^{T} and R^{X} have the same meanings as in the above formula (1). In particular, the structural unit A is preferably represented by, for example, the formula (A-1), (A-4), (A-5), (A-6), or (A-8).

The content of the structural unit A in the resin (when there are a plurality of types of structural unit A, the total content thereof is taken) is preferably 10 mol % or more, more preferably 20 mol % or more, and still more preferably 30 mol % or more based on all structural units constituting the resin. The content is preferably 80 mol % or less, more preferably 70 mol % or less, and still more preferably 60 mol % or less. When the content of the structural unit A is adjusted to within the above range, the sensitivity and CDU performance of the radiation-sensitive resin composition can be further improved.

### (Structural Unit B)

The structural unit B (hereinafter also referred to as “repeating unit B”) is a repeating unit containing an organic acid anion moiety and a sulfonium cation moiety containing an aromatic ring structure having a fluorine atom.

The structural unit B is a repeating unit derived from a monomer having a structure that is decomposed through exposure to light to generate an acid.

The structural unit B is preferably, for example, a repeating unit derived from a monomer represented by the following formula (2) or a monomer represented by the following formula (3).

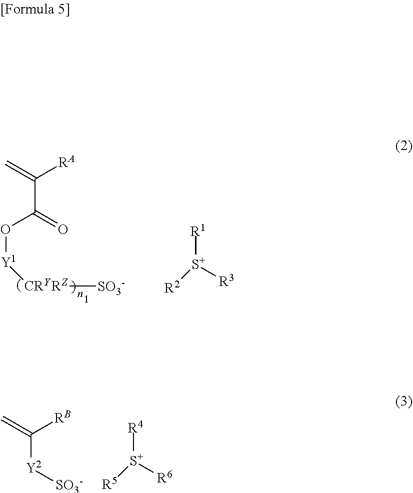

(In the formulas (2) and (3),
R^{A} and R^{B} are each a hydrogen atom, a fluorine atom, a methyl group, or a trifluoromethyl group;
R^{Y} and R^{Z} are independently a hydrogen atom, a fluorine atom, or a fluorinated hydrocarbon group, and at least one of R^{Y} and R^{Z} is a fluorine atom or a fluorinated hydrocarbon group; when there are a plurality of R^{Y}s and R^{Z}s, they may be the same or different;
n₁ is an integer of 1 to 20;
R¹ to R³ are independently a monovalent hydrocarbon group, and at least one of R¹ to R³ is an aromatic ring having a fluorine atom;
R⁴ to R⁶ are independently a monovalent hydrocarbon group, and at least one of R⁴ to R⁶ is an aromatic ring having a fluorine atom;
Y¹ is a single bond or —Y¹¹—C(═O)—O—; Y¹¹ is a divalent hydrocarbon group having 1 to 20 carbon atoms and optionally containing a heteroatom;
Y² is a single bond, a methylene group, an ethylene group, a phenylene group, a fluorinated phenylene group, —O—Y²¹—, —C(═O)—O—Y²¹—, or —C(═O)—NH—Y²¹—; Y²¹ is an alkanediyl group having 1 to 6 carbon atoms, an alkenediyl group having 2 to 6 carbon atoms, or a phenylene group, and may contain a carbonyl group, an ester linkage, an ether linkage, or a hydroxy group; the alkanediyl group having 1 to 6 carbon atoms, the alkenediyl group having 2 to 6 carbon atoms, and the phenylene group each may be substituted with a fluorine atom.)

In the formulas (2) and (3), R^{Y} and R^{Z} are independently a hydrogen atom, a fluorine atom, or a monovalent fluorinated hydrocarbon group having 1 to 20 carbon atoms, and at least one of R^{Y} and R^{Z} is a fluorine atom or a fluorinated hydrocarbon group. The hydrocarbon group constituting the monovalent fluorinated hydrocarbon group may be linear, branched, or cyclic, and examples thereof include alkyl groups such as a methyl group, an ethyl group, a n-propyl group, an isopropyl group, a n-butyl group, and a tert-butyl group; cycloalkyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups such as a vinyl group, an allyl group, a propenyl group, a butenyl group, a hexenyl group, and a cyclohexenyl group; aryl groups such as a phenyl group, a naphthyl group, and a thienyl group; and aralkyl groups such as a benzyl group, a 1-phenylethyl group, and a 2-phenylethyl group. Examples of the monovalent fluorinated hydrocarbon group include those in which some or all of hydrogen atoms of these hydrocarbon groups are replaced by a fluorine atom-containing group. when there are a plurality of R^{Y}s and R^{Z}s, they may be the same or different;

In the formulas (2) and (3), R¹ to R³ are independently a monovalent hydrocarbon group, provided that at least one of R¹ to R³ is an aromatic ring having a fluorine atom, and R⁴ to R⁶ are independently a monovalent hydrocarbon group, provided that at least one of R⁴ to R⁶ is an aromatic ring having a fluorine atom. In the present description, the “aromatic ring having a fluorine atom” refers to a structure in which some or all of hydrogen atoms contained in the aromatic ring are replaced by a fluorine atom or a fluorinated hydrocarbon group (preferably a perfluorohydrocarbon group). The monovalent hydrocarbon group may be linear, branched, or cyclic, and examples thereof include those the same as those disclosed as examples of the hydrocarbon group constituting the fluorinated hydrocarbon group in R^{Y} and R^{Z}, and an aryl group is preferable. Some of the hydrogen atoms of these groups may be replaced by a group containing a heteroatom such as an oxygen atom, a sulfur atom, a nitrogen atom, or a halogen atom. Any two or more of R¹ to R³ may be bonded to each other to form a ring together with the sulfur atom to which they are bonded, and any two or more of R⁴ to R⁶ may be bonded to each other to form a ring together with the sulfur atom to which they are bonded.

In the formula (2), when Y¹ is —Y¹¹—C(═O)—O—, examples of the divalent hydrocarbon group having 1 to 20 carbon atoms and optionally containing a heteroatom represented by Y¹¹ include, but are not limited to, those shown below.

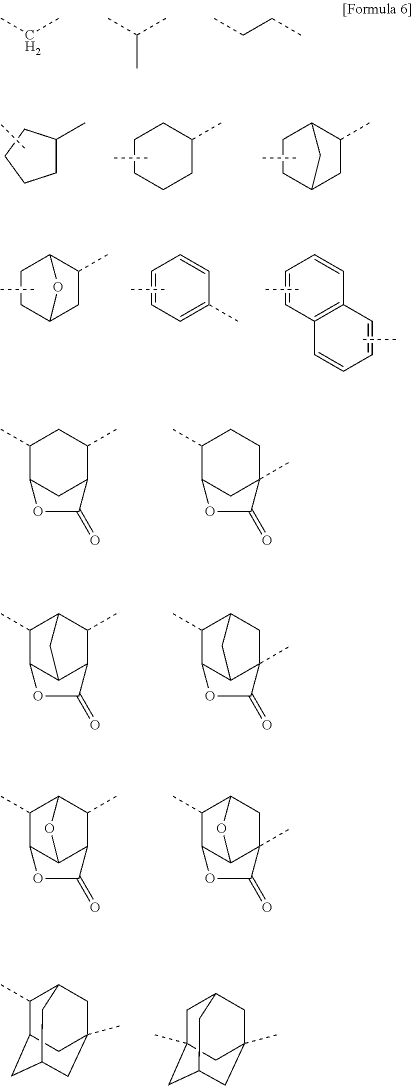

(In the formulas, the broken lines are bonds.)

In the formulas (2) and (3), any two of R¹ to R³ may be bonded to each other to form a ring together with the sulfur atom to which they are bonded, and any two of R⁴ to R⁶ may be bonded to each other to form a ring together with the sulfur atom to which they are bonded.

In the formulas (2) and (3), examples of specific structures of the sulfonium cation include, but are not limited to, those shown below.

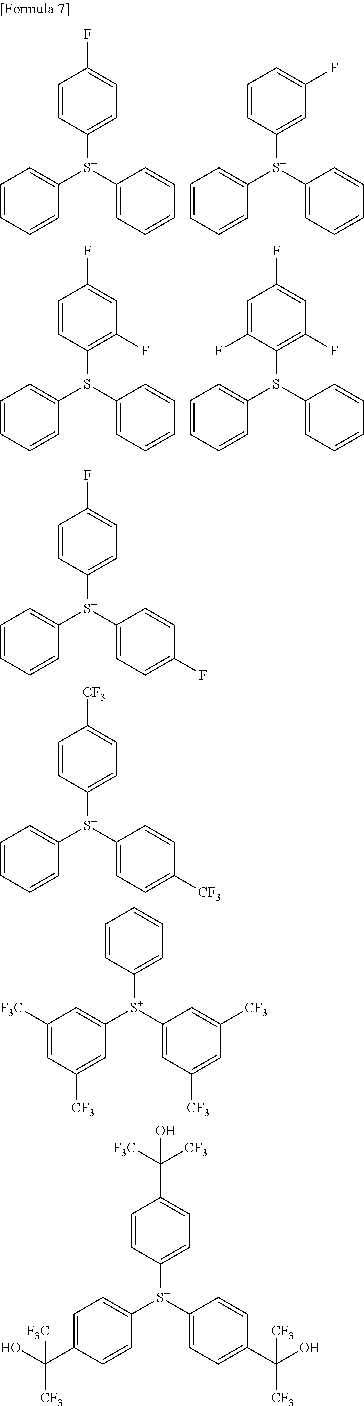

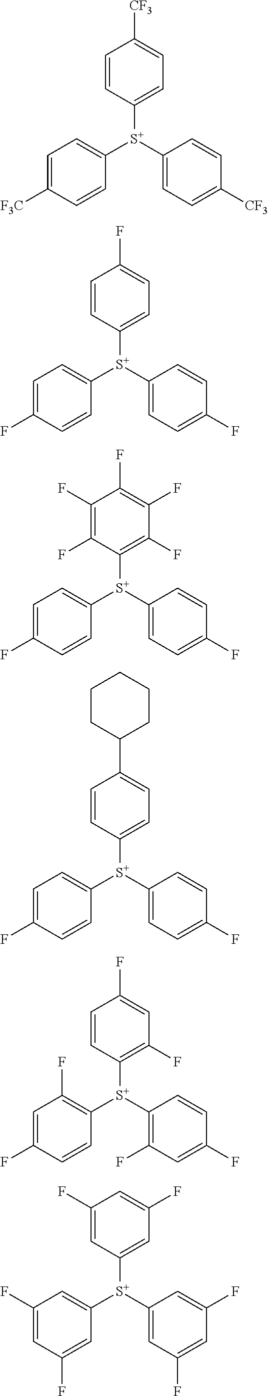 [SMILES: FC(F)(F)c1ccc([S+](c2ccc(C(F)(F)F)cc2)c2ccc(C(F)(F)F)cc2)cc1 ; Fc1ccc([S+](c2ccc(F)cc2)c2ccc(F)cc2)cc1 ; Fc1ccc([S+](c2ccc(F)cc2)c2c(F)c(F)c(F)c(F)c2F)cc1 ; Fc1ccc([S+](c2ccc(F)cc2)c2ccc(C3CCCCC3)cc2)cc1 ; Fc1ccc([S+](c2ccc(F)cc2F)c2ccc(F)cc2F)c(F)c1 ; Fc1cc(F)cc([S+](c2cc(F)cc(F)c2)c2cc(F)cc(F)c2)c1]

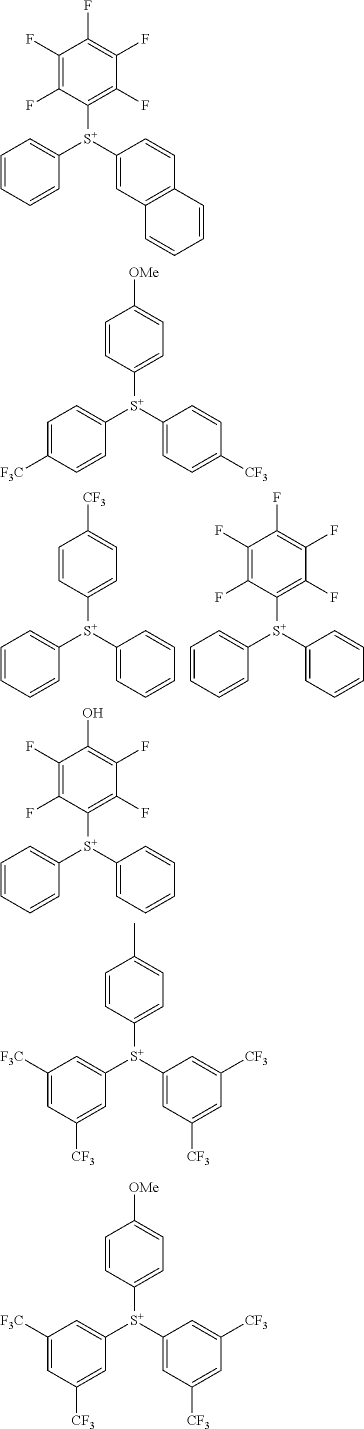

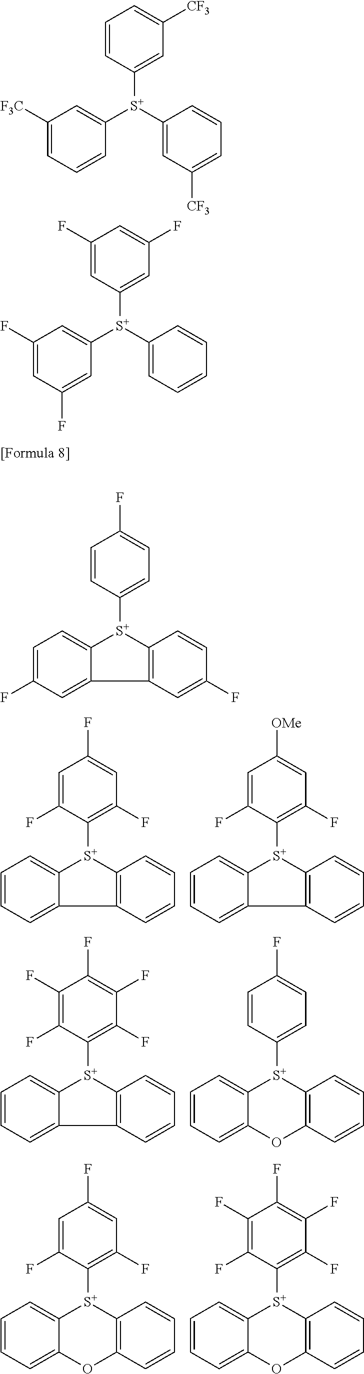

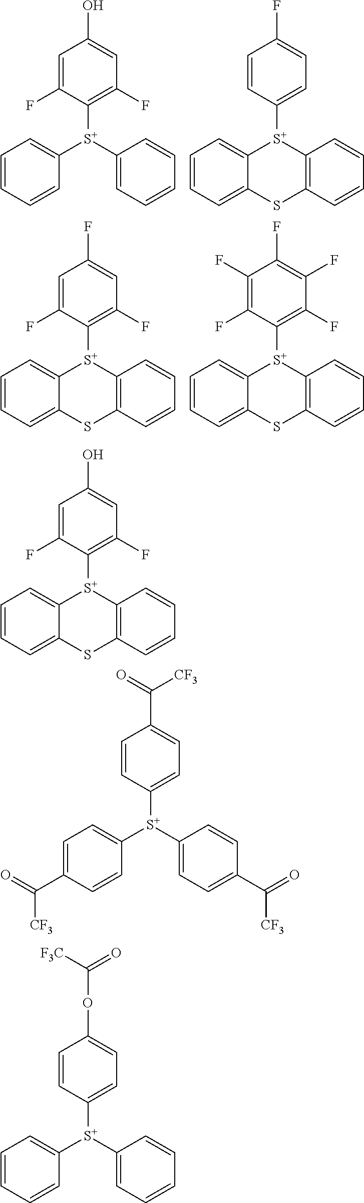

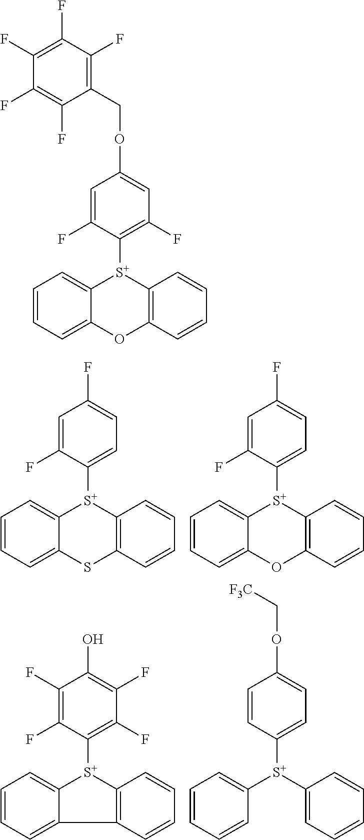 [SMILES: Fc1cc(OCc2c(F)c(F)c(F)c(F)c2F)cc(F)c1[S+]1c2ccccc2Oc2ccccc21 ; Fc1ccc([S+]2c3ccccc3Sc3ccccc32)c(F)c1 ; Fc1ccc([S+]2c3ccccc3Oc3ccccc32)c(F)c1 ; Oc1c(F)c(F)c(-[s+]2c3ccccc3c3ccccc32)c(F)c1F ; FC(F)(F)COc1ccc([S+](c2ccccc2)c2ccccc2)cc1]

The content of the structural unit B in the resin (when there are a plurality of types of structural unit B, the total content thereof is taken) is preferably 2 mol % or more, more preferably 3 mol % or more, still more preferably 4 mol % or more, and particularly preferably 5 mol % or more based on all structural units constituting the resin. The content is preferably 30 mol % or less, more preferably 25 mol % or less, still more preferably 20 mol % or less, and particularly preferably 15 mol % or less. When the content is adjusted to within the above range, a function as an acid generator can be sufficiently exhibited.

### (Structural Unit C)

The structural unit C (hereinafter also referred to as “repeating unit C”) is a repeating unit having an organic acid anion moiety and an onium cation moiety (however, it is different from the repeating unit B).

The structural unit C preferably contains, for example, a structural unit represented by the following formula (c1) (hereinafter also referred to as “structural unit c1”) or a structural unit represented by the following formula (c2) (hereinafter also referred to as “structural unit c2”).

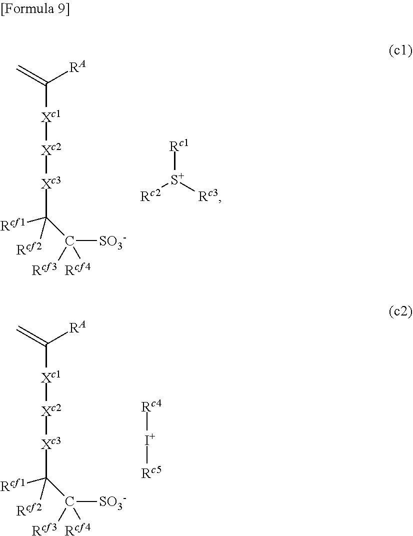

In the formulas, R^{A} is a hydrogen atom or a methyl group.

In the formulas, X^{c1} is a single bond or an ester group. X^{c2} is an alkylene group having 1 to 12 carbon atoms, a cycloalkylene group having 3 to 12 carbon atoms, or an arylene group having 6 to 10 carbon atoms. Some of the methylene groups constituting the alkylene group may be replaced by an ether group, an ester group, or a lactone ring-containing group. Some of the methylene groups constituting the cycloalkylene group may be replaced by an ether group or an ester group. At least one hydrogen atom contained in X^{c2} may be replaced by an iodine atom. X^{c3} is a single bond, an ether group, an ester group, an alkylene group having 1 to 12 carbon atoms, or a cycloalkylene group having 3 to 12 carbon atoms. Some of the methylene groups constituting the alkylene group and the cycloalkylene group may be replaced by an ether group or an ester group.

In the formulas, R^{cf1} to R^{cf4} are each independently a hydrogen atom, a fluorine atom, or a trifluoromethyl group, and at least one of R^{cf1} to R^{cf4} is a fluorine atom or a fluorinated hydrocarbon group.

In the formulas, R^{c1} to R^{c5} are each independently a monovalent hydrocarbon group having 1 to 20 carbon atoms and optionally containing a heteroatom, and R^{c1} and R^{c2} may be bonded to each other to form a ring together with the sulfur atom to which they are bonded.

Examples of the monovalent hydrocarbon group having 1 to 20 carbon atoms and optionally containing a heteroatom in R^{c1} to R^{c5} include the same monovalent hydrocarbon groups as those constituting R¹ to R⁶ in the formulas (2) and (3).

The repeating unit C is preferably, for example, a repeating unit derived from a monomer represented by the following formulas.

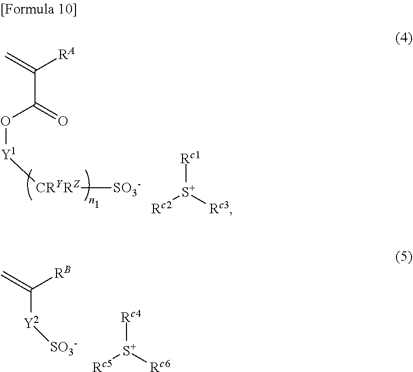

(In the formulas (4) and (5),
R^{A} and R^{B} are each a hydrogen atom, a fluorine atom, a methyl group, or a trifluoromethyl group;
R^{Y} and R^{Z} are independently fluorine atom or a fluorinated hydrocarbon group, and at least one of R^{Y} and R^{Z} is a fluorine atom or a fluorinated hydrocarbon group; when there are a plurality of R^{Y}s and R^{Z}s, they may be the same or different;
n₁ is an integer of 1 to 20;
R^{c1} to R^{c3} are independently a monovalent hydrocarbon group;
R^{c4} to R^{c6} are independently a monovalent hydrocarbon group;
Y¹ is a single bond or —Y¹¹—C(═O)—O—; Y¹¹ is a divalent hydrocarbon group having 1 to 20 carbon atoms and optionally containing a heteroatom;
Y² is a single bond, a methylene group, an ethylene group, a phenylene group, a fluorinated phenylene group, —O—Y²¹—, —C(═O)—O—Y²¹—, or —C(═O)—NH—Y²¹—; Y²¹ is an alkanediyl group having 1 to 6 carbon atoms, an alkenediyl group having 2 to 6 carbon atoms, or a phenylene group, and may contain a carbonyl group, an ester linkage, an ether linkage, or a hydroxy group.)

In the formulas (4) and (5), R^{c1} to R^{c3} are independently a monovalent hydrocarbon group, and R^{c4} to R^{c6} are independently a monovalent hydrocarbon group. The monovalent hydrocarbon group may be linear, branched, or cyclic, and examples thereof include those the same as those disclosed as examples of the hydrocarbon group constituting the fluorinated hydrocarbon group in R^{Y} and R^{Z}, and an aryl group is preferable. Some of the hydrogen atoms of these groups may be replaced by a group containing a heteroatom such as an oxygen atom, a sulfur atom, a nitrogen atom, or a halogen atom. Any two or more of R^{c1} to R^{c3} may be bonded to each other to form a ring together with the sulfur atom to which they are bonded, and any two or more of R^{c4} to R^{c6} may be bonded to each other to form a ring together with the sulfur atom to which they are bonded.

In the formulas (4) and (5), other moieties have the same meanings as the formulas (2) and (3).

The structural unit c1 and the structural unit c2 are preferably represented by the following formulas (c1-1) and (c2-1), respectively.

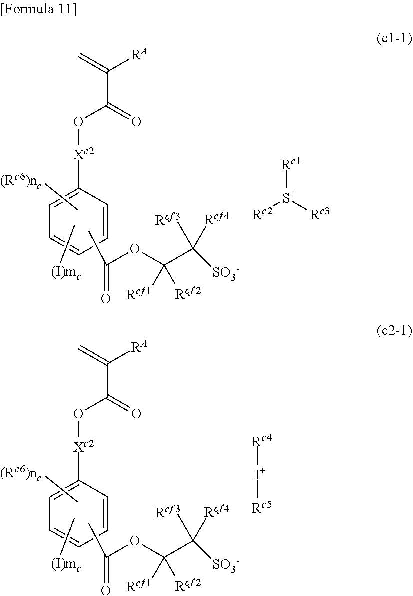

In the formulas, R^{A}, R^{c1} to R^{c5}, R^{cf1} to R^{cf4}, and X^{c2} have the same meanings as the formula (c1) or (c2).

In the formulas, R^{c6} is a linear, branched or cyclic alkyl group having 1 to 4 carbon atoms, a halogen atom other than iodine, a hydroxy group, a linear, branched or cyclic alkoxy group having 1 to 4 carbon atoms, or a linear, branched or cyclic alkoxycarbonyl group having 2 to 5 carbon atoms.

In the formulas, m_{c} is an integer of 0 to 4.

In the formulas, n_{c} is an integer of 0 to 3.

Examples of the organic acid anion moiety of the monomer that affords the structural unit c1 or the structural unit c2 include, but are not limited to, those shown below. While all of those shown below are organic acid anion moieties having an iodine-substituted aromatic ring structure, organic acid anion moieties having no iodine-substituted aromatic ring structure that can be suitably employed include structures in which the iodine atoms in the formulas shown below are replaced by an atom or group other than an iodine atom such as a hydrogen atom or other substituent.

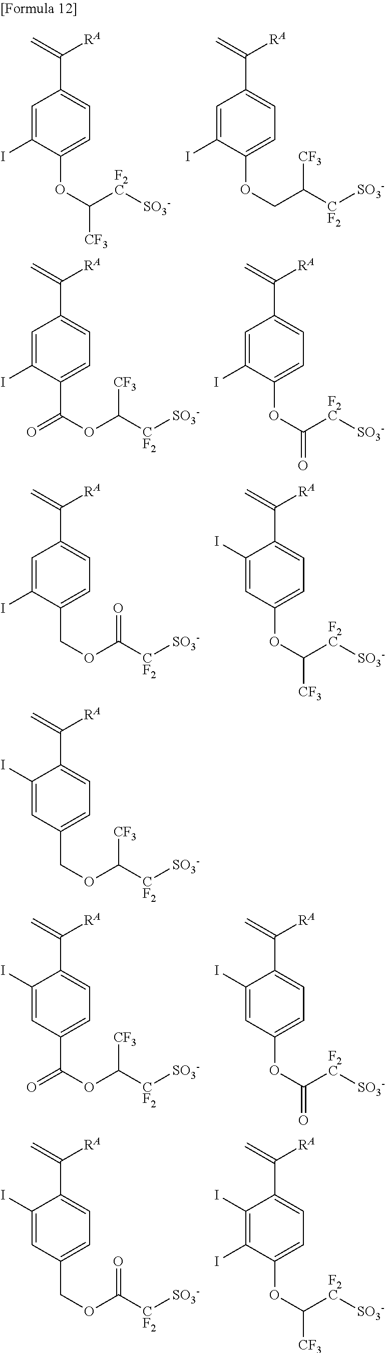

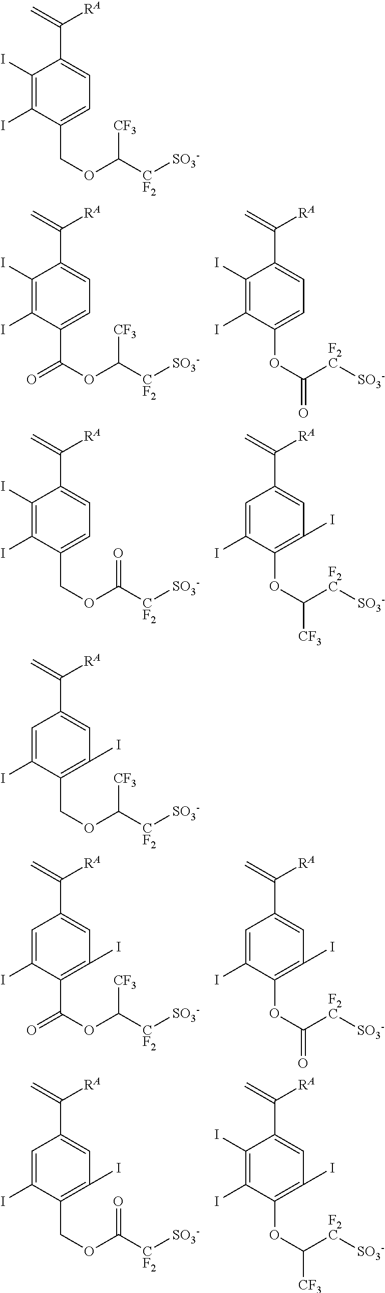

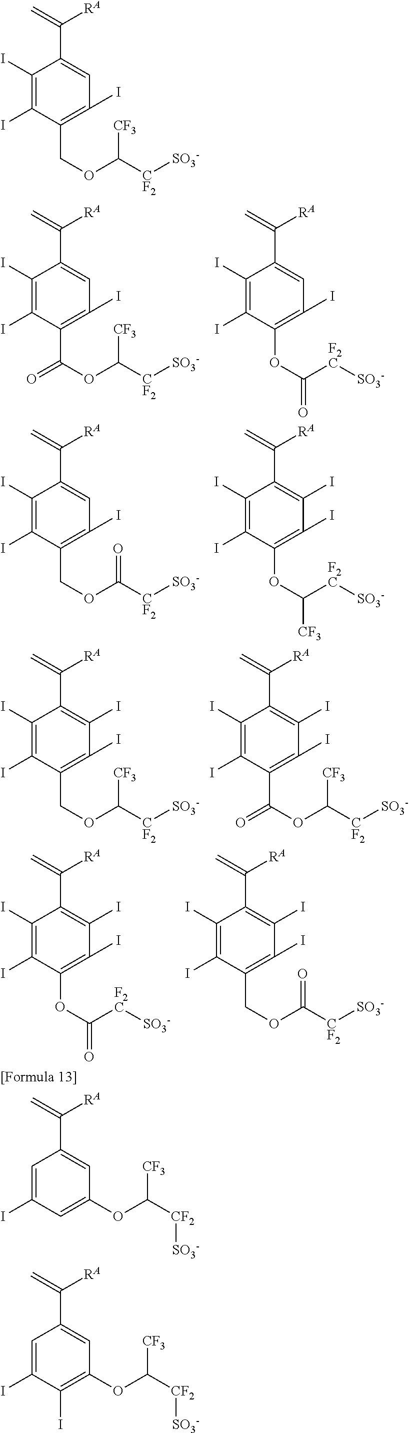

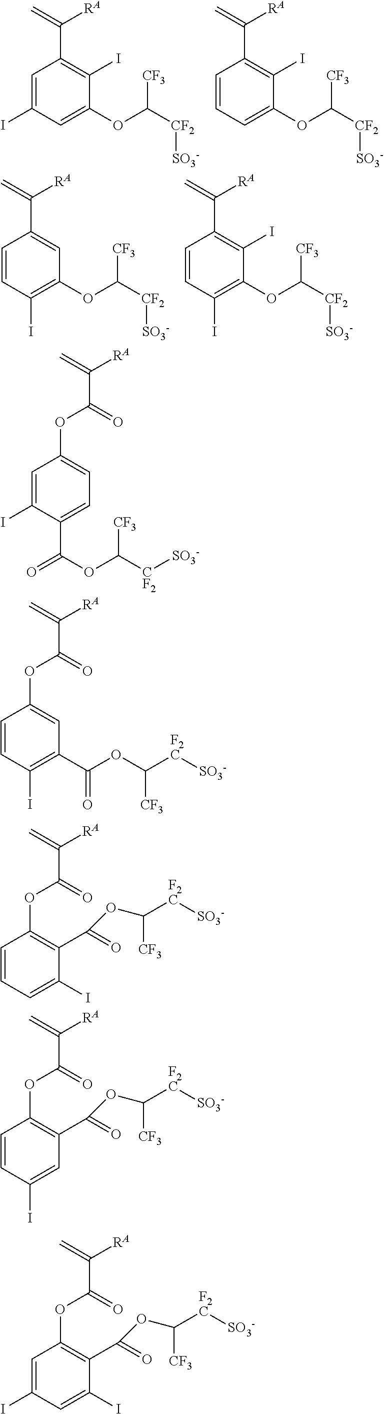

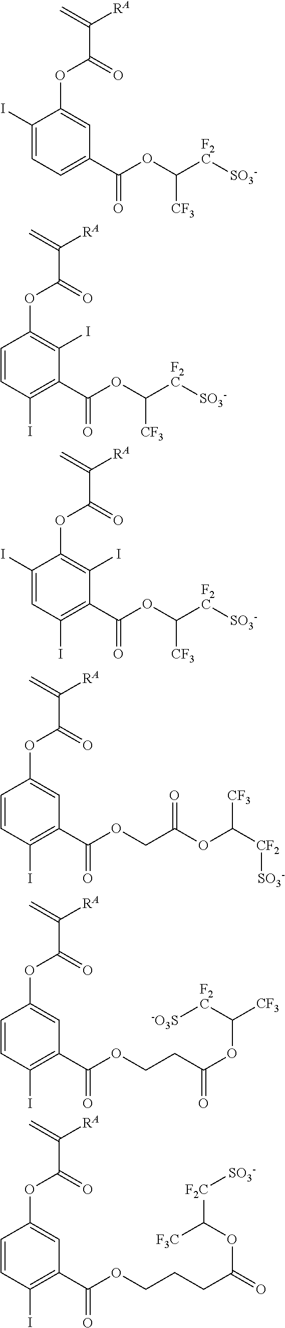

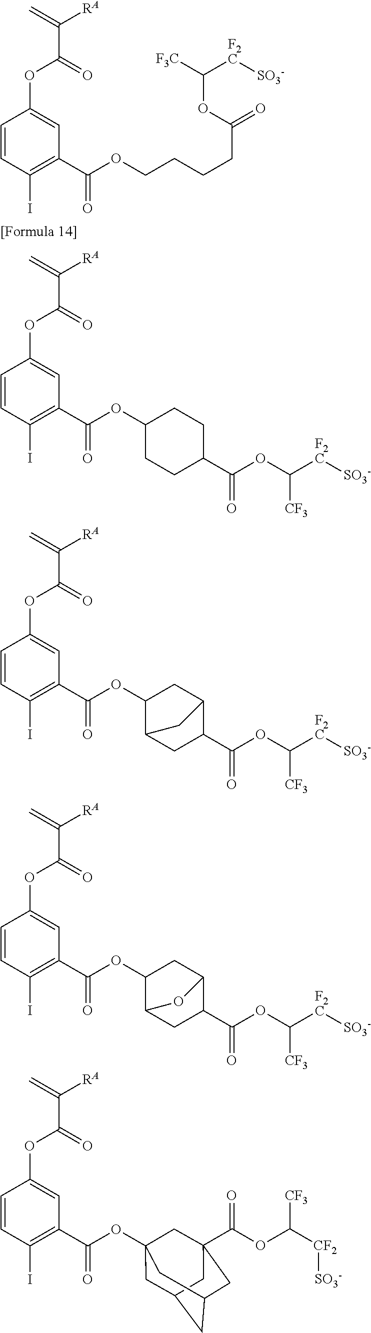

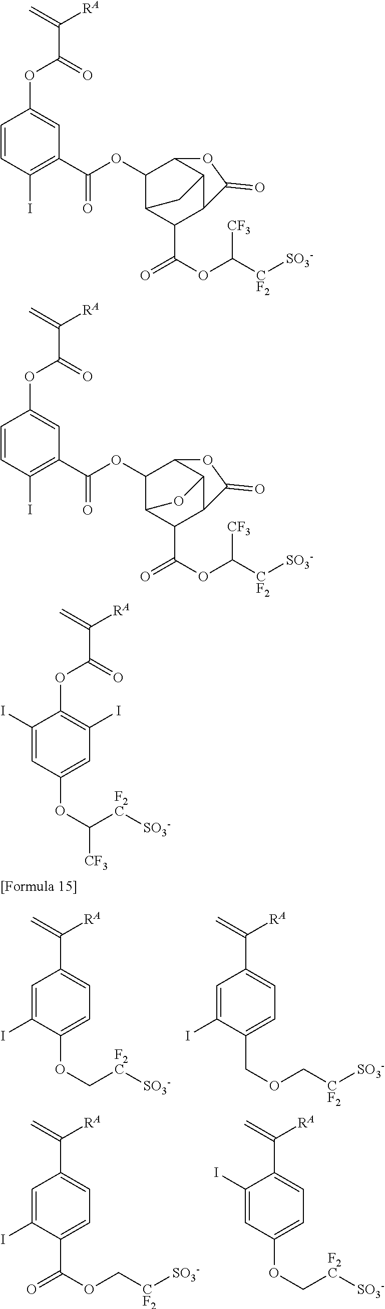

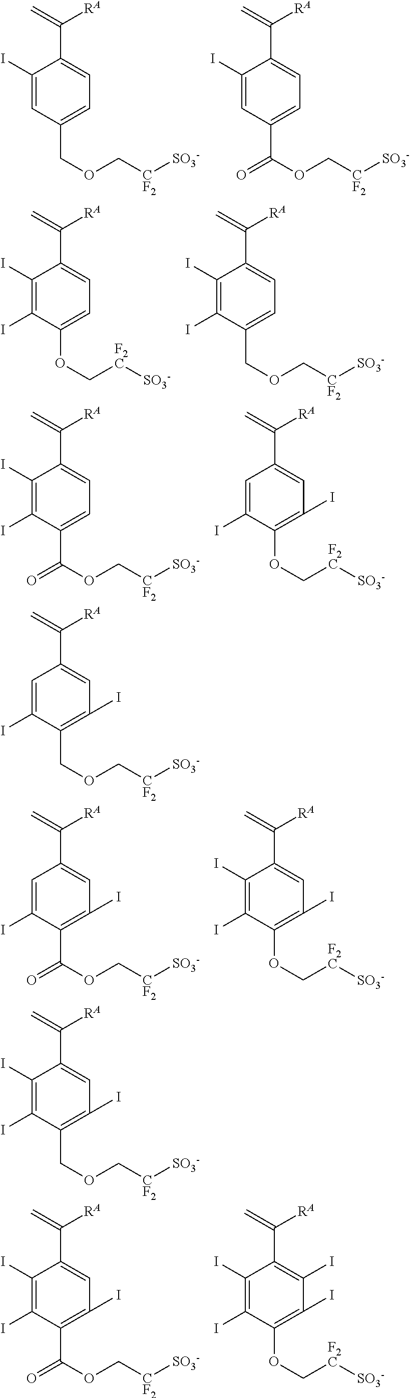

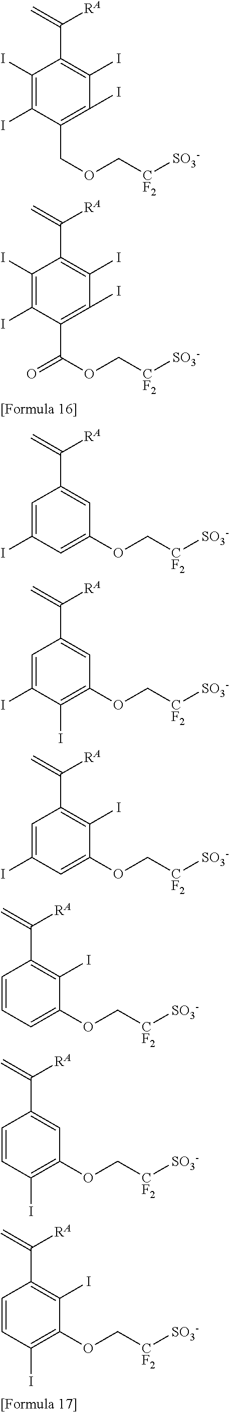

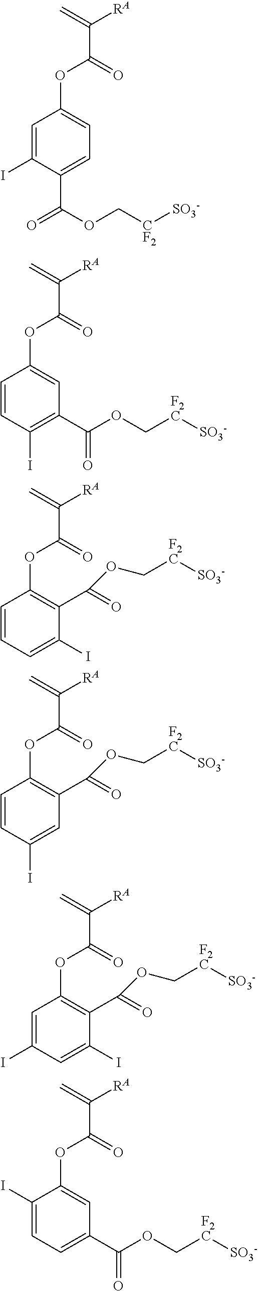

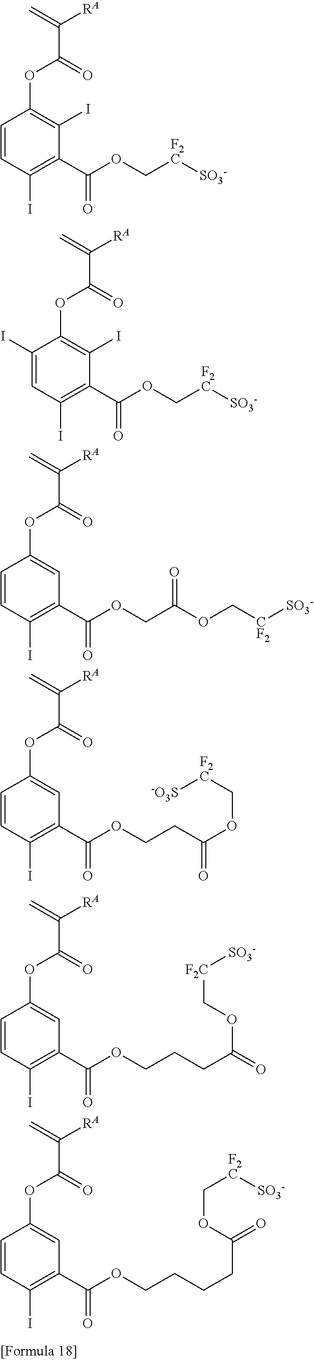

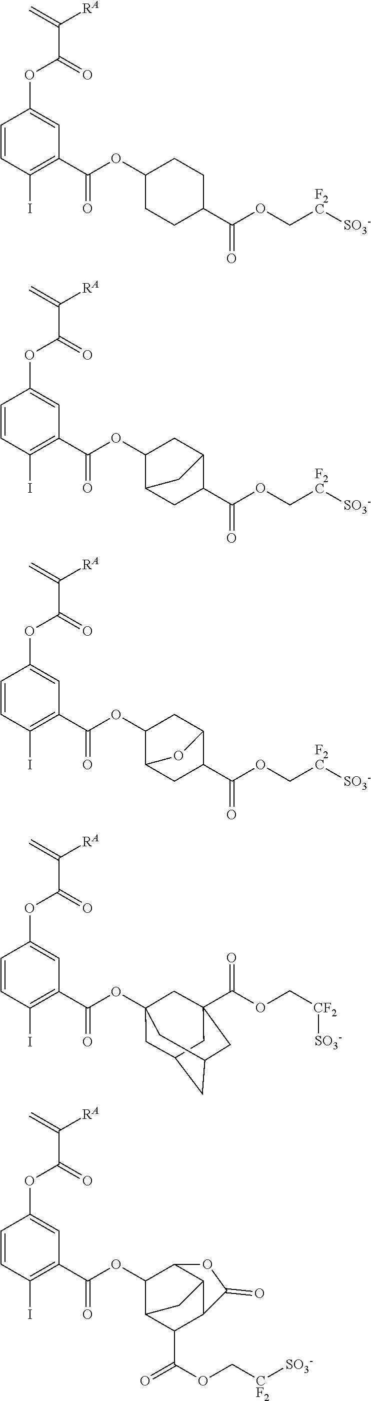

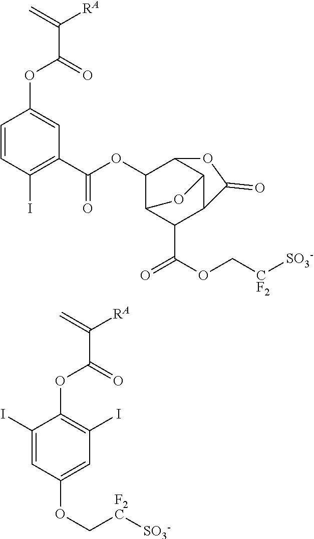

The onium cation moiety of the structural unit c1 is preferably represented by the following formula (Q-1).

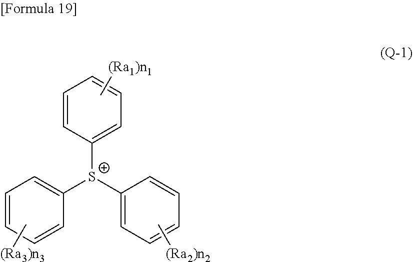

In the formula (Q-1), Ra1 and Ra2 each independently represent a substituent. n1 represents an integer of 0 to 5, and when n1 is 2 or more, the plurality of Ra1s may be the same or different. n2 represents an integer of 0 to 5, and when n2 is 2 or more, the plurality of Ra2s may be the same or different. n3 represents an integer of 0 to 5, and when n3 is 2 or more, the plurality of Ra3s may be the same or different. Ra3 represents a fluorine atom or a group having one or more fluorine atoms. Ra1 and Ra2 may be linked to each other to form a ring. When n1 is 2 or more, a plurality of Ra1's may be linked to each other to form a ring. When n2 is 2 or more, a plurality of Ra2's may be linked to each other to form a ring. When n1 is 1 or more and n2 is 1 or more, Ra1 and Ra2 may be linked to each other to form a ring (namely, a heterocyclic ring containing a sulfur atom).

The substituent represented by Ra1 and Ra2 is preferably an alkyl group, a cycloalkyl group, an alkoxy group, a cycloalkyloxy group, an alkoxycarbonyl group, an alkylsulfonyl group, a hydroxy group, a halogen atom, or a halogenated hydrocarbon group.

The alkyl group as Ra1 and Ra2 may be either linear or branched. As the alkyl group, those having 1 to 10 carbon atoms are preferable, and examples thereof include those disclosed as examples of the hydrocarbon group constituting the fluorinated hydrocarbon group in R^{Y} and R^{Z}. Among them, a methyl group, an ethyl group, a n-butyl group, and a t-butyl group are particularly preferable.

Examples of the cycloalkyl group as Ra1 and Ra2 include monocyclic or polycyclic cycloalkyl groups (preferably cycloalkyl groups having 3 to 20 carbon atoms), and examples thereof include a cyclopropyl group, a cyclobutyl group, a cyclopentyl group, a cyclohexyl group, a cycloheptyl group, a cyclooctyl group, a cyclododecanyl group, a cyclopentenyl group, a cyclohexenyl group, and a cyclooctadienyl group. Among these, a cyclopropyl group, a cyclopentyl group, a cyclohexyl group, a cyclohexyl group, a cycloheptyl group, and a cyclooctyl group are particularly preferable.

Examples of the alkyl group moiety of the alkoxy group as Ra1 and Ra2 include those listed above as the alkyl group as Ra1 and Ra2. As the alkoxy group, a methoxy group, an ethoxy group, a n-propoxy group, and a n-butoxy group are particularly preferable.

Examples of the cycloalkyl group moiety of the cycloalkyloxy group as Ra1 and Ra2 include those listed above as the cycloalkyl group as Ra1 and Ra2. As the cycloalkyloxy group, a cyclopentyloxy group and a cyclohexyloxy group are particularly preferable.

Examples of the alkoxy group moiety of the alkoxycarbonyl group as Ra1 and Ra2 include those listed above as the alkoxy group as Ra1 and Ra2. As the alkoxycarbonyl group, a methoxycarbonyl group, an ethoxycarbonyl group, and a n-butoxycarbonyl group are particularly preferable.

Examples of the alkyl group moiety of the alkylsulfonyl group as Ra1 and Ra2 include those listed above as the alkyl group as Ra1 and Ra2. Examples of the cycloalkyl group moiety of the cycloalkylsulfonyl group as Ra1 and Ra2 include those listed above as the cycloalkyl group as Ra1 and Ra2. As the alkylsulfonyl group or the cycloalkylsulfonyl group, a methanesulfonyl group, an ethanesulfonyl group, a n-propanesulfonyl group, a n-butanesulfonyl group, a cyclopentanesulfonyl group, and a cyclohexanesulfonyl group are particularly preferable.

Each of the groups Ra1 and Ra2 may further have a substituent. Examples of the substituent include a halogen atom such as a fluorine atom (preferably a fluorine atom), a hydroxy group, a carboxy group, a cyano group, a nitro group, an alkoxy group, a cycloalkyloxy group, an alkoxyalkyl group, a cycloalkyloxyalkyl group, an alkoxycarbonyl group, a cycloalkyloxycarbonyl group, an alkoxycarbonyloxy group, and a cycloalkyloxycarbonyloxy group.

Examples of the halogen atom as Ra1 and Ra2 include a fluorine atom, a chlorine atom, a bromine atom, and an iodine atom, and a fluorine atom is preferable.

As the halogenated hydrocarbon group as Ra1 and Ra2, a halogenated alkyl group is preferable. Examples of the alkyl group and the halogen atom constituting the halogenated alkyl group include those described above. Among them, a fluorinated alkyl group is preferable, and CF₃ is more preferable.

As described above, Ra1 and Ra2 may be linked to each other to form a ring (namely, a heterocyclic ring containing a sulfur atom). In this case, it is preferable that Ra1 and Ra2 are bonded to each other to form a single bond or a divalent linking group. Examples of the divalent linking group include —COO—, —OCO—, —CO—, —O—, —S—, —SO—, —SO₂—, an alkylene group, a cycloalkylene group, an alkenylene group, and combinations of two or more thereof, and those having of 20 or less carbon atoms in total are preferable. When Ra1 and Ra2 are linked to each other to form a ring, it is preferable that Ra1 and Ra2 are bonded to each other to form —COO—, —OCO—, —CO—, —O—, —S—, —SO—, —SO₂—, or a single bond. Among them, it is more preferable to form —O—, —S—, or a single bond, and it is particularly preferable to form a single bond. When n1 is 2 or more, a plurality of Ra1's may be linked to each other to form a ring, and when n2 is 2 or more, a plurality of Ra2's may be linked to each other to form a ring. Examples thereof include an embodiment in which two Ra1's are linked to each other to form a naphthalene ring together with a benzene ring to which they are bonded.

Ra3 is a fluorine atom or a group having one or more fluorine atoms. Examples of the group having a fluorine atom include groups in which an alkyl group, a cycloalkyl group, an alkoxy group, a cycloalkyloxy group, an alkoxycarbonyl group, and an alkylsulfonyl group as Ra1 and Ra2 are substituted with a fluorine atom. Among them, fluorinated alkyl groups are suitable, CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉, and CH₂CH₂C₄F₉ are more suitable, and CF₃ is particularly suitable.

Ra3 is preferably a fluorine atom or CF₃, and more preferably a fluorine atom.

n1 and n2 are each independently preferably an integer of 0 to 3, and preferably an integer of 0 to 2.

n3 is preferably an integer of 1 to 3, and more preferably 1 or 2.

(n1+n2+n3) is preferably an integer of 0 to 15, more preferably an integer of 1 to 9, still more preferably an integer of 2 to 6, and particularly preferably an integer of 3 to 6. When (n1+n2+n3) is 1, it is preferable that n3=1 and Ra3 is a fluorine atom or CF₃. When (n1+n2+n3) is 2, a combination in which n1=n3=1 and Ra1 and Ra3 are each independently a fluorine atom or CF₃ and a combination in which n3=2 and Ra3 is a fluorine atom or CF₃ are preferable. When (n1+n2+n3) is 3, a combination in which n1=n2=n3=1 and Ra1 to Ra3 are each independently a fluorine atom or CF₃ is preferable. When (n1+n2+n3) is 4, a combination in which n1=n3=2 and Ra1 and Ra3 are each independently a fluorine atom or CF₃ is preferable. When (n1+n2+n3) is 5, a combination in which n1=n2=1 and n3=3 and Ra1 to Ra3 are each independently a fluorine atom or CF₃, a combination in which n1=n2=2 and n3=1 and Ra1 to Ra3 are each independently a fluorine atom or CF₃, and a combination in which n3=5 and Ra3 are each independently a fluorine atom or CF₃ are preferable. When (n1+n2+n3) is 6, a combination in which n1=n2=n3=2 and Ra1 to Ra3 are each independently a fluorine atom or CF₃ is preferable.

Examples of the onium cation moiety represented by the formula (Q-1) include structures in which a fluorine atom, a fluorinated hydrocarbon group, and a monovalent group having a fluorine atom in the structures of the sulfonium cations disclosed above as examples of the formulas (2) and (3) are replaced by a hydrogen atom or other substituent.

When the onium cation moiety of the structural unit c2 contains an aromatic ring structure having a fluorine atom, the onium cation moiety is preferably a diaryliodonium cation having one or more fluorine atoms. Among them, the onium cation moiety is preferably represented by the following formula (Q-2).

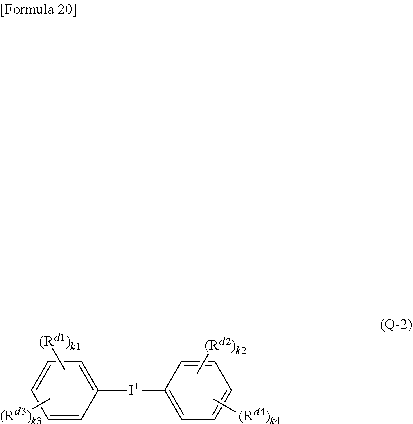

In the formula, R^{d1} and R^{d2} are each independently a substituted or unsubstituted linear or branched alkyl group, alkoxy group or alkoxycarbonyl group having 1 to 12 carbon atoms, a substituted or unsubstituted aromatic hydrocarbon group having 6 to 12 carbon atoms, or a nitro group; R^{d3} and R^{d4} are each independently a fluorine atom or a group having a fluorine atom; k1 and k2 are each independently an integer of 0 to 5, k3 and k4 are each independently an integer of 0 to 5, provided that (k1+k3) and (k2+k4) are each 5 or less, and (k3+k4) is an integer of 1 to 10; and when there are a plurality of R^{d1}'s to R^{d4}'s, the plurality of R^{d1}'s to R^{d4}'s may be the same or different, respectively.

Examples of the alkyl group, the alkoxy group, and the alkoxycarbonyl group represented by R^{d1} and R^{d2} and the group having a fluorine atom represented by R^{d3} and R^{d4} include the same groups as those represented by the above formula (Q-1).

Examples of the monovalent aromatic hydrocarbon groups having 6 to 12 carbon atoms include aryl groups such as a phenyl group, a tolyl group, a xylyl group, and a naphthyl group; and aralkyl groups such as a benzyl group and a phenethyl group.

Examples of the substituent of the respective groups include halogen atoms such as a fluorine atom, a chlorine atom, a bromine atom, and an iodine atom; a hydroxy group; a carboxy group; a cyano group; a nitro group; an alkyl group, an alkoxy group, an alkoxycarbonyl group, an alkoxycarbonyloxy group, an acyl group, an acyloxy group, or a group in which a hydrogen atom of these groups has been substituted with a halogen atom; and an oxo group (═O).

k1 and k2 are each preferably 0 to 2, and more preferably 0 or 1. k3 and k4 are each preferably 1 to 3, and more preferably 1 or 2. (k3+k4) is an integer of 1 to 10, preferably an integer of 1 to 6, more preferably an integer of 1 to 4, and still more preferably 1 or 2.

Examples of such an onium cation moiety represented by the formula (Q-2) include those shown below. While all of those shown below are iodonium cation moieties containing an aromatic ring structure having a fluorine atom, structures in which a fluorine atom or CF₃ in the following formulas is replaced by an atom or group other than a fluorine atom such as a hydrogen atom or other substituent can be suitably employed as an onium cation moiety containing no aromatic ring structure having a fluorine atom.

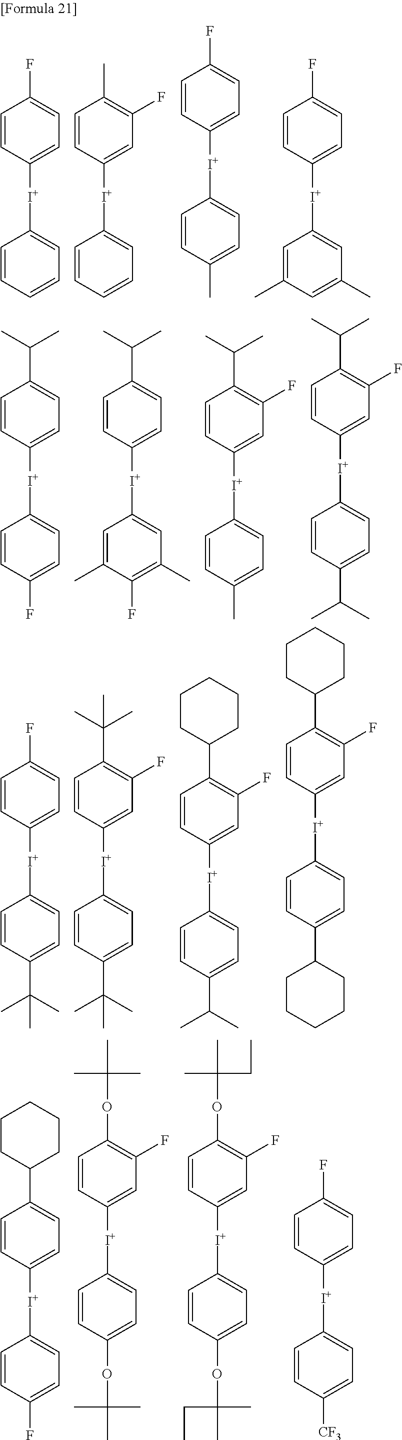

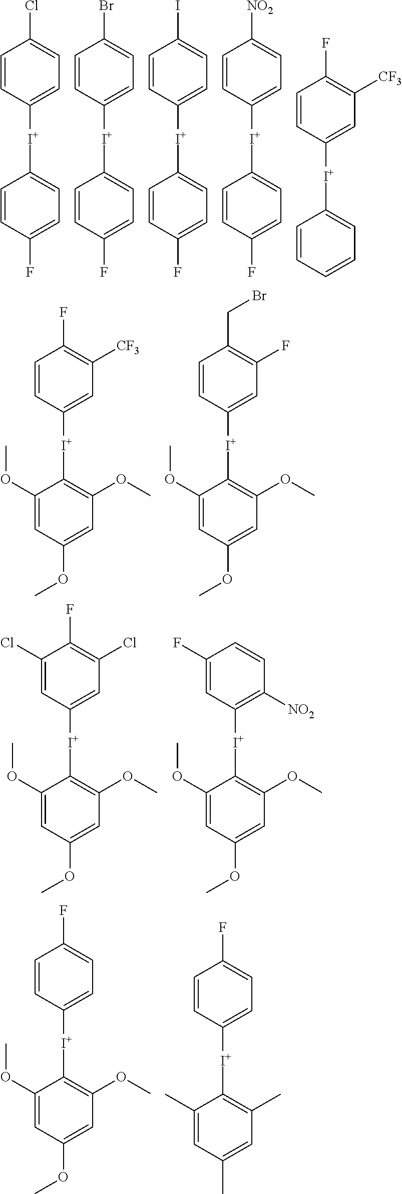

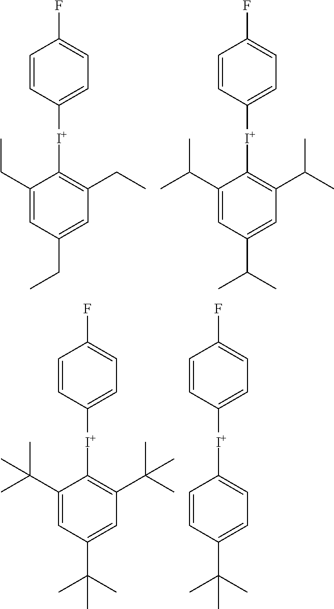 [SMILES: CCc1cc(CC)c([I+]c2ccc(F)cc2)c(CC)c1 ; CC(C)c1cc(C(C)C)c([I+]c2ccc(F)cc2)c(C(C)C)c1 ; CC(C)(C)c1cc(C(C)(C)C)c([I+]c2ccc(F)cc2)c(C(C)(C)C)c1 ; CC(C)(C)c1ccc([I+]c2ccc(F)cc2)cc1]

The content of the structural unit C in the resin (when there are a plurality of types of structural unit C, the total content thereof is taken) is preferably 2 mol % or more, more preferably 3 mol % or more, still more preferably 4 mol % or more, and particularly preferably 5 mol % or more based on all structural units constituting the resin. The content is preferably 30 mol % or less, more preferably 25 mol % or less, still more preferably 20 mol % or less, and particularly preferably 15 mol % or less. When the content is adjusted to within the above range, a function as an acid generator can be sufficiently exhibited.

### (Structural Unit D)

The structural unit D is a structural unit having a phenolic hydroxy group or a structural unit that affords a phenolic hydroxy group due to the action of an acid. In the present invention, a phenolic hydroxy group generated through deprotection due to the action of an acid generated by exposure to light is also included as the phenolic hydroxy group of the structural unit D. When the resin contains the structural unit D, the solubility thereof in a developer can be more appropriately adjusted, and as a result, the sensitivity and the like of the radiation-sensitive resin composition can be further improved. When KrF excimer laser light, EUV, electron beam or the like is used as radiation to be applied in an exposure step in a method for forming a resist pattern, the structural unit D contributes to improvement in etching resistance and improvement in the difference in solubility in a developer (namely, dissolution contrast) between an exposed area and an unexposed area. In particular, the resin can be suitably applied to pattern formation using exposure with radiation having a wavelength of 50 nm or less such as electron beam or EUV. The structural unit D is preferably represented by the following formula (D).

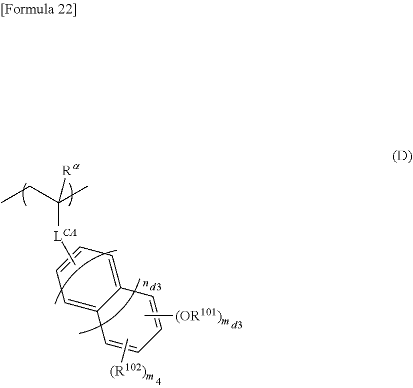

(in the formula (D),
R^{α} is a hydrogen atom, a fluorine atom, a methyl group, or a trifluoromethyl group;
L^{CA} is a single bond, —COO—*, or —O—; * is a bond on the aromatic ring side;
R¹⁰¹ is a hydrogen atom or a protective group that is deprotected by the action of an acid; when there are a plurality of R¹⁰¹'s, the plurality of R¹⁰¹'s are the same or different from each other;
R¹⁰² is a cyano group, a nitro group, an alkyl group, a fluorinated alkyl group, an alkoxycarbonyloxy group, an acyl group, or an acyloxy group;
n_{d3} is an integer of 0 to 2, m_{3d} is an integer of 1 to 8, and m_{d4} is an integer of 0 to 8, provided that 1≤m_{d3}+m_{d4}≤2n_{d3}+5 is satisfied.)

The R^{α} is preferably a hydrogen atom or a methyl group from the viewpoint of the copolymerizability of a monomer that affords the structural unit D.

L^{CA} is preferably a single bond or —COO—*.

Examples of the protecting group that is deprotected due to the action of the acid represented by R¹⁰¹ include groups represented by the following formulas (AL-1) to (AL-3).

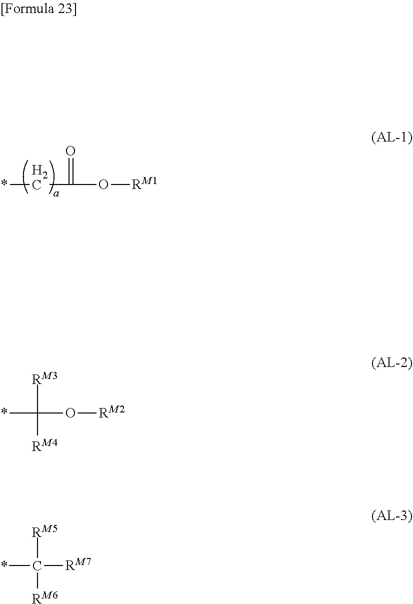

In the formulas (AL-1) and (AL-2), R^{M1} and R^{M2} are monovalent hydrocarbon groups, and may contain a heteroatom such as an oxygen atom, a sulfur atom, a nitrogen atom, or a fluorine atom. The monovalent hydrocarbon group may be linear, branched, or cyclic, and is preferably an alkyl group having 1 to 40 carbon atoms, and more preferably an alkyl group having 1 to 20 carbon atoms. In the formula (AL-1), a is an integer of 0 to 10, and preferably an integer of 1 to 5. In the formulas (AL-1) to (AL-3), * is a bond to another moiety.

In the formula (AL-2), R^{M3} and R^{M4} are each independently a hydrogen atom or a monovalent hydrocarbon group, and may contain a heteroatom such as an oxygen atom, a sulfur atom, a nitrogen atom, or a fluorine atom. The monovalent hydrocarbon group may be linear, branched, or cyclic, and is preferably an alkyl group having 1 to 20 carbon atoms. Any two of R^{M2}, R^{M3}, and R^{M4} may be bonded to each other to form a ring having 3 to 20 carbon atoms together with the carbon atom or the carbon atom and the oxygen atom to which they are bonded. The ring is preferably a ring having 4 to 16 carbon atoms, and particularly preferably an alicyclic ring.

In the formula (AL-3), R^{M5}, R^{M6}, and R^{M7} are each independently a monovalent hydrocarbon group, and may contain a heteroatom such as an oxygen atom, a sulfur atom, a nitrogen atom, or a fluorine atom. The monovalent hydrocarbon group may be linear, branched, or cyclic, and is preferably an alkyl group having 1 to 20 carbon atoms. Any two of R^{M5}, R^{M6}, and R^{M7} may be bonded to each other to form a ring having 3 to 20 carbon atoms together with the carbon atom to which they are bonded. The ring is preferably a ring having 5 to 16 carbon atoms, and particularly preferably an alicyclic ring.

Among them, the protecting group that is deprotected due to the action of an acid is preferably a group represented by the formula (AL-3).

Examples of the alkyl group in R¹⁰² include linear or branched alkyl groups having 1 to 8 carbon atoms such as a methyl group, an ethyl group, and a propyl group. Examples of the fluorinated alkyl group include linear or branched fluorinated alkyl groups having 1 to 8 carbon atoms such as a trifluoromethyl group and a pentafluoroethyl group. Examples of the alkoxycarbonyloxy group include chain or alicyclic alkoxycarbonyloxy groups having 2 to 16 carbon atoms such as a methoxycarbonyloxy group, a butoxycarbonyloxy group, and an adamantylmethyloxycarbonyloxy group. Examples of the acyl group include aliphatic or aromatic acyl groups having 2 to 12 carbon atoms such as an acetyl group, a propionyl group, a benzoyl group, and an acryloyl group. Examples of the acyloxy group include aliphatic or aromatic acyloxy groups having 2 to 12 carbon atoms such as an acetyloxy group, a propionyloxy group, a benzoyloxy group, and an acryloyloxy group.

The n_{d3} is preferably 0 or 1, and more preferably 0.

The m₃ is preferably an integer of 1 to 3, and more preferably 1 or 2.

The m₄ is preferably an integer of 0 to 3, and more preferably an integer of 0 to 2.

As the structural unit D, structural units represented by the following formulas (D-1) to (D-10) (hereinafter also referred to as “structural units (D-1) to (D-10)”) and the like are preferable.

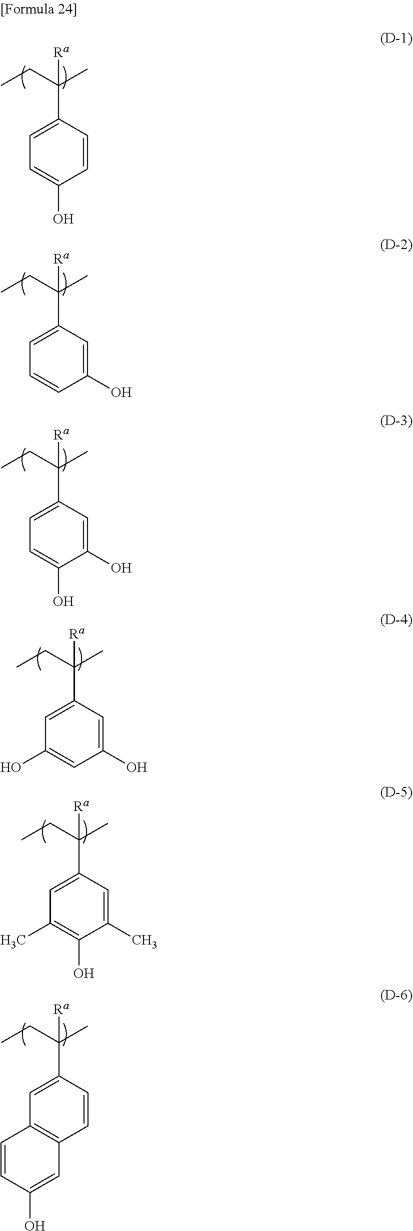

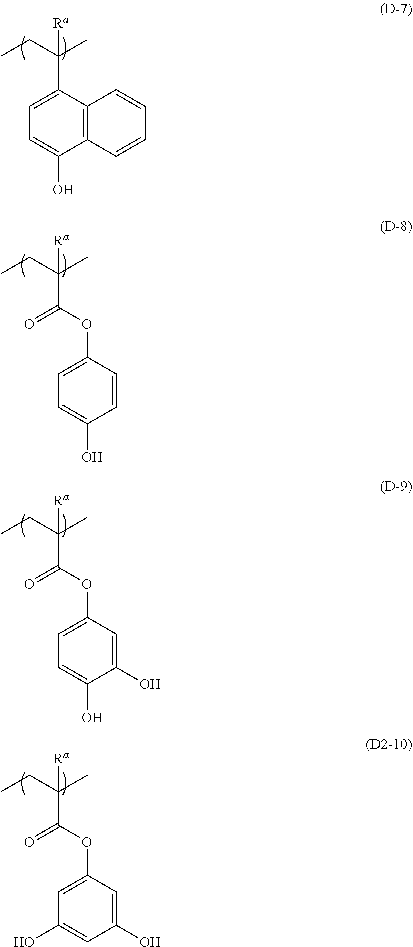

In the formulas (D-1) to (D-10), R^{α} is the same as in the above formula (D).

Among them, the structural units (D-1) to (D-4), (D-6) and (D-8) are preferable.

The content of the structural unit D (when there are a plurality of types of structural unit D, the total content thereof is taken) is preferably 5 mol % or more, more preferably 8 mol % or more, still more preferably 10 mol % or more, and particularly preferably 15 mol % or more based on all structural units constituting the resin. The content is preferably 60 mol % or less, more preferably 50 mol % or less, still more preferably 40 mol % or less, and particularly preferably 35 mol % or less. When the content of the structural unit C is adjusted to within the above range, the sensitivity, CDU performance, and resolution of the radiation-sensitive resin composition can be further improved.

In the case of polymerizing a monomer having a phenolic hydroxy group such as hydroxystyrene, it is preferable to polymerize the monomer with the phenolic hydroxy group protected by a protecting group such as an alkali-dissociable group and then deprotect it by hydrolysis to obtain a structural unit D.

### (Structural Unit E)

The structural unit E is a structural unit containing at least one structure selected from the group consisting of a lactone structure, a cyclic carbonate structure, and a sultone structure. When the base resin further has the structural unit E, the solubility of the base resin in a developer can be adjusted, and as a result, the lithographic performance, such as resolution, of the radiation-sensitive resin composition can be improved. In addition, the adhesion between a resist pattern formed from the base resin and a substrate can be improved.

Examples of the structural unit E include structural units represented by the following formulas (T-1) to (T-10).

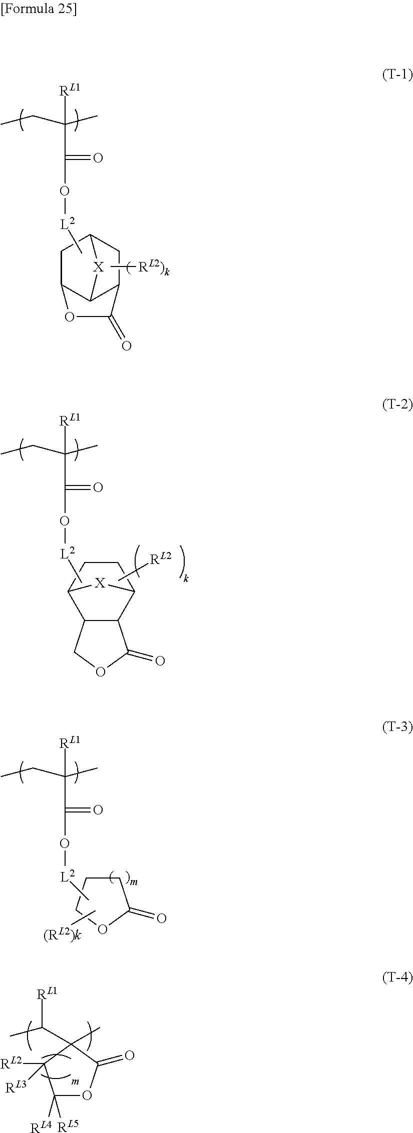

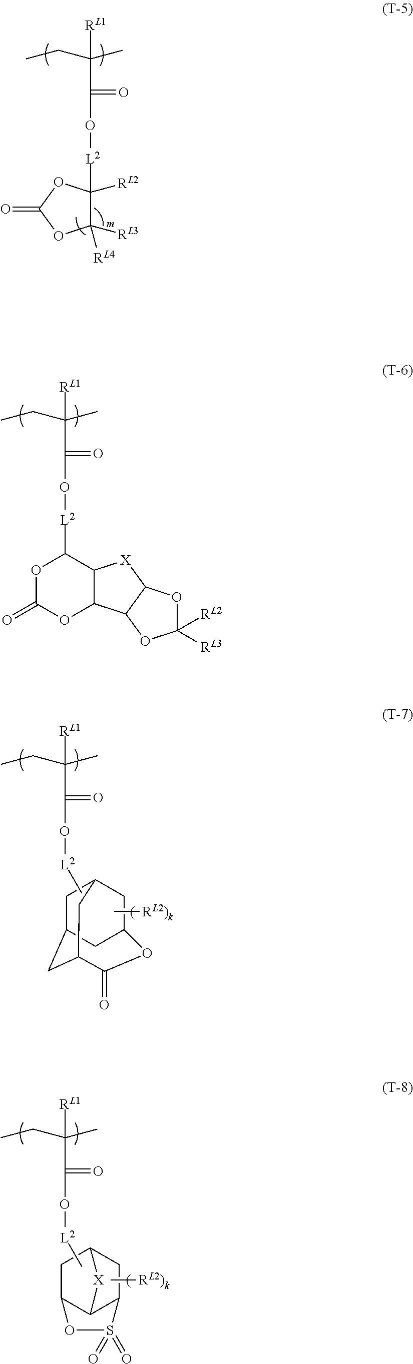

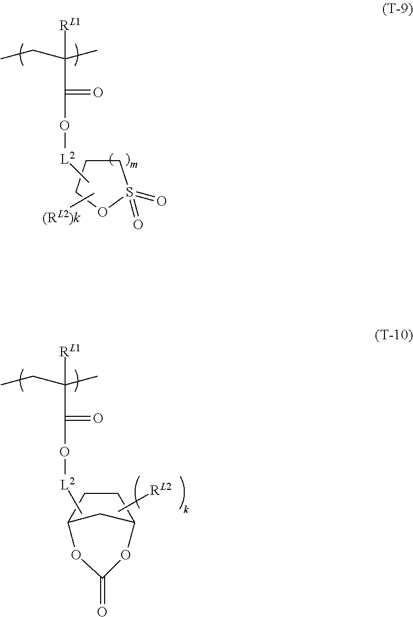

In the above formula, R^{L1} is a hydrogen atom, a fluorine atom, a methyl group, or a trifluoromethyl group. R^{L2} to R^{L5} are each independently a hydrogen atom, an alkyl group having 1 to 4 carbon atoms, a cyano group, a trifluoromethyl group, a methoxy group, a methoxycarbonyl group, a hydroxy group, a hydroxymethyl group, or a dimethylamino group. R^{L4} and R^{L5} may be combined with each other and constitute a divalent alicyclic group having 3 to 8 carbon atoms together with the carbon atom to which they are bonded. L² is a single bond or a divalent linking group. X is an oxygen atom or a methylene group. k is an integer of 0 to 3. m is an integer of 1 to 3.

The divalent alicyclic group having 3 to 8 carbon atoms composed of the R^{L4} and the R^{L5} combined together as well as the carbon atoms to which the R^{L4} and the R^{L5} are bonded is not particularly limited as long as it is a group formed by removing two hydrogen atoms from the same carbon atom contained in a carbon ring of a monocyclic or polycyclic alicyclic hydrocarbon having the aforementioned number of carbon atoms. The group may be either a monocyclic hydrocarbon group or a polycyclic hydrocarbon group, and the polycyclic hydrocarbon group may be either a bridged alicyclic hydrocarbon group or a fused alicyclic hydrocarbon group, and may be either a saturated hydrocarbon group or an unsaturated hydrocarbon group. It is to be noted that the fused alicyclic hydrocarbon group refers to a polycyclic alicyclic hydrocarbon group in which two or more alicyclic rings share a side (a bond between two adjacent carbon atoms).

Among the monocyclic alicyclic hydrocarbon groups, as the saturated hydrocarbon group, a cyclopentanediyl group, a cyclohexanediyl group, a cycloheptanediyl group, a cyclooctanediyl group, and the like are preferable, and as the unsaturated hydrocarbon group, a cyclopentenediyl group, a cyclohexenediyl group, a cycloheptenediyl group, a cyclooctenediyl group, a cyclodecenediyl group, and the like are preferable. As the polycyclic alicyclic hydrocarbon group, bridged alicyclic saturated hydrocarbon groups are preferable, and for example, a bicyclo[2.2.1]heptane-2,2-diyl group (norbornane-2,2-diyl group), a bicyclo[2.2.2]octane-2,2-diyl group, and a tricyclo[3.3.1.1^{3,7}]decane-2,2-diyl group (adamantane-2,2-diyl group) are preferable. One or more hydrogen atoms on the alicyclic group may be replaced by a hydroxy group.

Examples of the divalent linking group represented by L² include a divalent linear or branched hydrocarbon group having 1 to 10 carbon atoms, a divalent alicyclic hydrocarbon group having 4 to 12 carbon atoms, and a group composed of one or more among these hydrocarbon groups and at least one group among —CO—, —O—, —NH—, and —S.

Among them, the structural unit E is preferably a structural unit containing a lactone structure, more preferably a structural unit containing a norbornane lactone structure, and still more preferably a structural unit derived from norbornane lactone-yl (meth)acrylate.

The structural unit E is preferably a structural unit represented by the following formula (6):

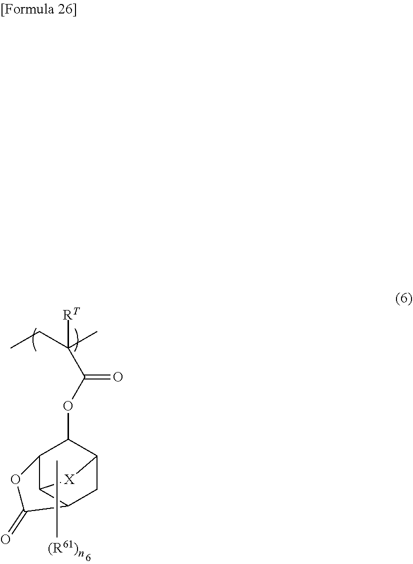

(In the formula (6),
X is a methanediyl group substituted or unsubstituted with R⁶¹, or —O—;
R^{T} is a hydrogen atom, a fluorine atom, a methyl group, or a trifluoromethyl group;
R⁶¹ is a monovalent hydrocarbon group having 1 to 20 carbon atoms; when there are a plurality of R⁶¹s, they may be the same or different; and
n₆ is an integer of 0 to 3.)

In the formula (6), R⁶¹ is independently in each occurrence a monovalent hydrocarbon group. The monovalent hydrocarbon group may be linear, branched, or cyclic, and examples thereof include the same groups as those disclosed as examples of the hydrocarbon group constituting the fluorinated hydrocarbon group in R^{Y} and R^{Z}, and an alkyl group is preferable. Some of the hydrogen atoms of these groups may be replaced by a group containing a heteroatom such as an oxygen atom, a sulfur atom, a nitrogen atom, or a halogen atom.

Examples of the formula (6) include structural units represented by the following formulas (6-1) to (6-2).

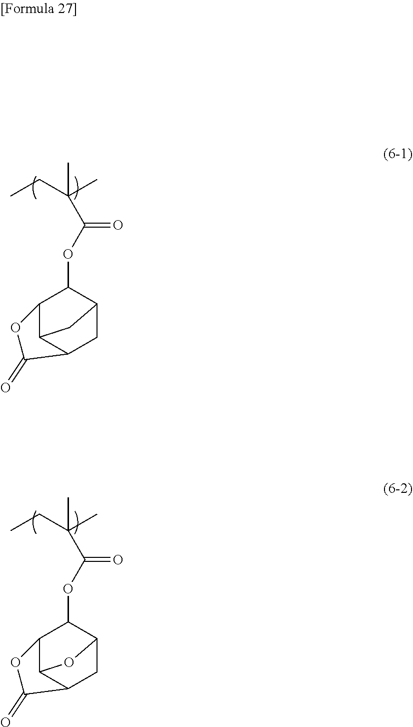

The content of the structural unit E is preferably 5 mol % or more, more preferably 10 mol % or more, and still more preferably 20 mol % or more based on all structural units constituting the base resin. The content is preferably 60 mol % or less, more preferably 50 mol % or less, and still more preferably 40 mol % or less. When the content of the structural unit E is adjusted to within the range, the lithographic performance, such as resolution, of the radiation-sensitive resin composition and the adhesion between a resist patter to be formed and a substrate can be further improved.

### (Method for Synthesizing Resin)

The resin as a base resin can be synthesized by, for example, subjecting monomers that will afford structural units to a polymerization reaction in an appropriate solvent using a publicly known radical polymerization initiator or the like.

The molecular weight of the resin as a base resin is not particularly limited, and the weight average molecular weight (Mw) as determined by Gel Permeation Chromatography (GPC) relative to standard polystyrene is preferably 1,000 or more, more preferably 2,000 or more, still more preferably 3,000 or more, and particularly preferably 4,000 or more. The Mw of the high fluorine-containing resin is preferably 50,000 or less, more preferably 30,000 or less, still more preferably 15,000 or less, and particularly preferably 12,000 or less. When the Mw of the resin is within the above range, a resulting resist film is good in heat resistance and developability.

The ratio (Mw/Mn) of Mw to the number average molecular weight (Mn) of the resin as a base resin as determined by GPC relative to standard polystyrene is usually 1 or more and 5 or less, preferably 1 or more and 3 or less, and more preferably 1 or more and 2 or less.

The Mw and the Mn of a resin in the present description are values measured using gel permeation chromatography (GPC) under the following conditions.
GPC column: two G2000HXL, one G3000HXL, one G4000HXL (all manufactured by Tosoh Corporation) Column temperature: 40° C.
Elution solvent: tetrahydrofuran
Flow rate: 1.0 mL/min
Sample concentration: 1.0% by mass
Amount of sample injected: 100 μL
Detector: differential refractometer
Standard substance: monodisperse polystyrene

The content of the resin is preferably 70% by mass or more, more preferably 75% by mass or more, and still more preferably 80% by mass based on the total solid content of the radiation-sensitive resin composition.

### <Other Resins>

The radiation-sensitive resin composition of the present embodiment may contain a resin having a higher content by mass of fluorine atoms than the base resin as described above (hereinafter also referred as “high fluorine-containing resin”) as other resin. When the radiation-sensitive resin composition contains the high fluorine-containing resin, the high fluorine-containing resin can be localized in the surface layer of a resist film compared to the base resin, and as a result, the state of the surface of the resist film and the component distribution in the resist film can be controlled to a desired state.

The high fluorine-containing resin preferably has, for example, one or more of the structural unit A through the structural unit E in the above-described base resin, as necessary, and have a structural unit represented by the following formula (f0) (hereinafter also referred to as “structural unit F”).

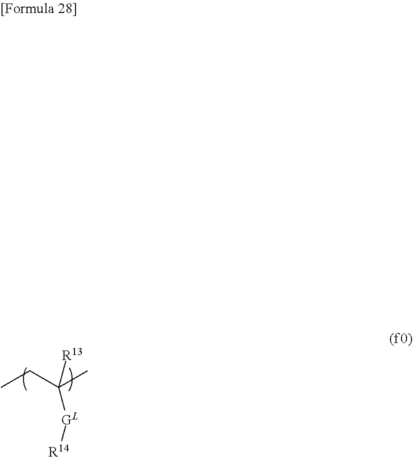

In the above formula (f0), R¹³ is a hydrogen atom, a methyl group, or a trifluoromethyl group. G is a single bond, an oxygen atom, a sulfur atom, —COO—, —SO₂ONH—, —CONH—, or —OCONH—. R¹⁴ is a monovalent fluorinated chain hydrocarbon group having 1 to 20 carbon atoms or a monovalent fluorinated alicyclic hydrocarbon group having 3 to 20 carbon atoms.

As the R¹³, a hydrogen atom and a methyl group are preferable from the viewpoint of the copolymerizability of a monomer that affords the structural unit F, and a methyl group is more preferable.

As the G^{L}, a single bond and —COO— are preferable from the viewpoint of the copolymerizability of a monomer that affords the structural unit F, and —COO— is more preferable.

Examples of the monovalent fluorinated chain hydrocarbon group having 1 to 20 carbon atoms represented by R¹⁴ include groups in which some or all of the hydrogen atoms in the linear or branched chain alkyl group having 1 to 20 carbon atoms are substituted with fluorine atoms.

Examples of the monovalent fluorinated alicyclic hydrocarbon group having 3 to 20 carbon atoms represented by R¹⁴ include monovalent fluorinated alicyclic hydrocarbon groups having 3 to 20 carbon atoms in which some or all of the hydrogen atoms of a mono- or polycyclic hydrocarbon group are substituted with fluorine atoms.

As the R¹⁴, fluorinated chain hydrocarbon groups are preferable, fluorinated alkyl groups are more preferable, and a 2,2,2-trifluoroethyl group, a 1,1,1,3,3,3-hexafluoropropyl group, a 5,5,5-trifluoro-1,1-diethylpentyl group, and a 1,1,1,2,2,3,3-heptafluoro-6-methylheptan-4-yl group are still more preferable.

When the high fluorine-containing resin contains the structural unit F, the content of the structural unit F is preferably 50 mol % or more, more preferably 60 mol % or more, and still more preferably 70 mol % or more based on all structural units constituting the high fluorine-containing resin. The content is preferably 100 mol % or less, more preferably 95 mol % or less, and still more preferably 90 mol % or less. When the content of the structural unit F is adjusted to within the above range, the content by mass of fluorine atoms in the high fluorine-containing resin can more appropriately be adjusted and the localization in the surface layer of a resist film can be further promoted.

The high fluorine-containing resin may have a fluorine atom-containing structural unit represented by the following formula (f-1) (hereinafter also referred to as structural unit f) in addition to the structural unit F. When the high fluorine-containing resin has the structural unit f, solubility in an alkaline developer is improved, and the occurrence of development defects can be suppressed.

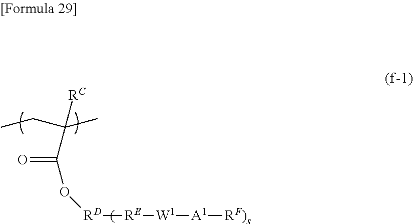

The structural unit f is roughly divided into two cases: a case where it has (x) an alkali-soluble group, and a case where it has (y) a group that is dissociated by the action of an alkali to increase the solubility in an alkaline developer (hereinafter also simply referred to as “alkali-dissociable group”). Commonly in (x) and (y), in the above formula (f-1), R^{C} is a hydrogen atom, a fluorine atom, a methyl group, or a trifluoromethyl group. R^{D} is a single bond, a hydrocarbon group having 1 to 20 carbon atoms with the valency of (s+1), a structure in which an oxygen atom, a sulfur atom, —NR^{dd}—, a carbonyl group, —COO— or —CONH— is connected to the terminal on R^{E} side of the hydrocarbon group, or a structure in which some of the hydrogen atoms in the hydrocarbon group are substituted with organic groups having a heteroatom. R^{dd} is a hydrogen atom or a monovalent hydrocarbon group having 1 to 10 carbon atoms. s is an integer of 1 to 3.

When the structural unit f has (x) an alkali-soluble group, RF is a hydrogen atom, and A¹ is an oxygen atom, —COO—* or —SO₂O—*. * indicates a site that bonds to R^{F}. W¹ is a single bond, a hydrocarbon group having 1 to 20 carbon atoms, or a divalent fluorinated hydrocarbon group. When A¹ is an oxygen atom, W¹ is a fluorinated hydrocarbon group having a fluorine atom or a fluoroalkyl group on the carbon atom to which A¹ is bonded. R^{E} is a single bond or a divalent organic group having 1 to 20 carbon atoms. When s is 2 or 3, a plurality of R^{E}'s, W¹'s, A¹'s, and R^{F}'s may be the same or different, respectively. When the structural unit f has (x) an alkali-soluble group, affinity to an alkaline developer can be increased, and development defects can be suppressed. As the structural unit f having (x) an alkali-soluble group, a case where A¹ is an oxygen atom and W¹ is a 1,1,1,3,3,3-hexafluoro-2,2-methanediyl group is particularly preferable.

When the structural unit f has (y) an alkali-dissociable group, R^{F} is a monovalent organic group having 1 to 30 carbon atoms, and A¹ is an oxygen atom, —NR^{aa}, —COO—* or —SO₂O—*. R^{aa} is a hydrogen atom or a monovalent hydrocarbon group having 1 to 10 carbon atoms. * indicates a site that bonds to R^{F}. W¹ is a single bond or a divalent fluorinated hydrocarbon group having 1 to 20 carbon atoms. R^{E} is a single bond or a divalent organic group having 1 to 20 carbon atoms. When A¹ is —COO—* or —SO₂O—*, W¹ or R^{F} has a fluorine atom on a carbon atom bonded to A¹ or on a carbon atom adjacent thereto. When A¹ is an oxygen atom, W¹ and R^{E} are single bonds, R^{D} is a structure in which a carbonyl group is bonded to a terminal on the R^{E} side of a hydrocarbon group having 1 to 20 carbon atoms, and R^{F} is an organic group having a fluorine atom. When s is 2 or 3, a plurality of R^{E}'s, W¹'s, A¹'s, and R^{F}'s may be the same or different, respectively. When the structural unit f has (y) an alkali-dissociable group, the surface of a resist film changes from hydrophobic to hydrophilic in an alkali development step. As a result, the affinity to a developer can be greatly increased, and development defects can be more efficiently suppressed. As the structural unit f having (y) an alkali-dissociable group, a structural unit in which A¹ is —COO—*, and R^{F}, W¹, or both of them have a fluorine atom is particularly preferable.

As the R^{C}, a hydrogen atom and a methyl group are preferable from the viewpoint of the copolymerizability of a monomer that affords the structural unit f, and a methyl group is more preferable.

When R^{E} is a divalent organic group, a group having a lactone structure is preferable, a group having a polycyclic lactone structure is more preferable, and a group having a norbornanelactone structure is still more preferable.

When the high fluorine-containing resin has the structural unit f, the content of the structural unit f is preferably 10 mol % or more, more preferably 20 mol % or more, still more preferably 30 mol % or more, and further preferably 35 mol % or more based on all structural units constituting the high fluorine-containing resin. The upper limit of the content is preferably 90 mol % or less, more preferably 75 mol % or less, still more preferably 60 mol % or less. When the content ratio of the structural unit f is adjusted to within the above range, the water repellency of a resist film during immersion exposure can be further improved.

The Mw of the high fluorine-containing resin is preferably 1,000 or more, more preferably 2,000 or more, still more preferably 3,000 or more, and particularly preferably 5,000 or more. The Mw is preferably 50,000 or less, more preferably 30,000 or less, still more preferably 20,000 or less, and particularly preferably 15,000 or less.

The Mw/Mn of the high fluorine-containing resin is usually 1 or more, and more preferably 1.1 or more. The Mw/Mn is usually 5 or less, preferably 3 or less, more preferably 2.5 or less, and still more preferably 2.2 or less.

The content of the high fluorine-containing resin is preferably 1 part by mass or more, more preferably 2 parts by mass or more, and still more preferably 3 parts by mass or more based on 100 parts by mass of the base resin (when a radiation-sensitive acid generating resin and a resin are contained, the total amount of them is taken as the basis). The content is preferably 20 parts by mass or less, more preferably 15 parts by mass or less, and still more preferably 10 parts by mass or less. When the content of the high fluorine-containing resin is adjusted to within the above range, the high fluorine-containing resin can be more effectively localized in the surface layer of a resist film, and as a result, the elusion of a top portion of a pattern is controlled during development and the rectangularity of a pattern can be enhanced. The radiation-sensitive resin composition may contain one type or two or more types of high fluorine-containing resins.

### (Method for Synthesizing High Fluorine-Containing Resin)

The high fluorine-containing resin can be synthesized by the same method as the method for synthesizing a base resin described above.

### <Onium Salt>

The onium salt is a component that contains an organic acid anion moiety and an onium cation moiety and generates an acid through exposure to light. When at least part of the onium cation moiety in the onium salt contains an aromatic ring structure having a fluorine atom, it is possible to achieve increased sensitivity due to improvement in acid generation efficiency and exhibition of CDU performance due to acid diffusion controllability.

The mode of incorporation of the onium salt in the radiation-sensitive resin composition is not particularly limited, but the onium salt is preferably at least one member selected from the group consisting of a radiation-sensitive acid generating resin containing a structural unit having the organic acid anion moiety and the onium cation moiety, a radiation-sensitive acid generator containing the organic acid anion moiety and the onium cation moiety, and an acid diffusion controlling agent containing the organic acid anion moiety and the onium cation moiety and being to generate an acid having a pKa higher than an acid to be generated from the radiation-sensitive acid generator through irradiation with radiation. Differences between these functions will be described below.

The acid generated through the exposure to the onium salt is considered to have two functions in the radiation-sensitive resin composition depending on the strength of the acid. Examples of the first function include a function that causes the acid generated through the exposure to dissociate an acid-dissociable group of a structural unit when the resin contains the structural unit having the acid-dissociable group, to generate a carboxy group or the like. An onium salt having the first function is referred to as a radiation-sensitive acid generator. Examples of the second function include a function that controls, by salt exchange, the diffusion of the acid generated from the radiation-sensitive acid generator in the unexposed area without substantially dissociating the acid-dissociable group of the resin under a pattern formation condition using the radiation-sensitive resin composition. An onium salt having the second function is referred to as an acid diffusion controlling agent. The acid generated from the acid diffusion controlling agent can be said to be an acid relatively weaker (acid having a higher pKa) than the acid to be generated from the radiation-sensitive acid generator. Whether an onium salt functions as a radiation-sensitive acid generator or an acid diffusion controlling agent depends on the energy required for dissociating the acid-dissociable group of the resin and the acidity of the onium salt. The mode of incorporation of the radiation-sensitive acid generator in the radiation-sensitive resin composition may be a mode in which the onium salt structure is present alone as a compound (released from a polymer), a mode in which the onium salt structure is incorporated as a part of a polymer, or both of these modes. A form in which an onium salt structure is incorporated as a part of a polymer is particularly referred to as a radiation-sensitive acid generating resin.

When the radiation-sensitive resin composition contains the radiation-sensitive acid generator or a radiation-sensitive acid generating resin, the polarity of the resin in an exposed area increases, and as a result, when the developer is an aqueous alkaline solution, the resin in the exposed area is soluble in the developer, and on the other hand, when the developer is an organic solvent, the resin in the exposed area is hardly soluble in the developer.

When the radiation-sensitive resin composition contains the acid diffusion controlling agent, diffusion of an acid in an unexposed area can be controlled, and a resist pattern further superior in pattern developability and CDU performance can be formed.

In the radiation-sensitive resin composition, it is just required that the onium cation moiety in at least one member selected from the group consisting of the radiation-sensitive acid generating resin, the radiation-sensitive acid generator, and the acid diffusion controlling agent contains an aromatic ring structure having a fluorine atom.

Even in any mode of incorporation of the onium salt, the organic acid anion moiety preferably has at least one type of anion selected from the group consisting of a sulfonate anion, a carboxylate anion, and a sulfonimide anion. The onium cation is preferably at least one selected from the group consisting of a sulfonium cation and an iodonium cation. When the onium salt has a combination of these structures, the above-described function can be efficiently exhibited.

Examples of the acid to be generated through the exposure include acids that generate sulfonic acid, carboxylic acid, and sulfonimide through exposure with correspondence to the organic acid anion.

Examples of an onium salt that affords a sulfonic acid through exposure include:
(1) a compound in which one or more fluorine atoms or fluorinated hydrocarbon groups are bonded to a carbon atom adjacent to a sulfonate anion, and
(2) a compound in which neither fluorine atom nor fluorinated hydrocarbon group is bonded to a carbon atom adjacent to a sulfonate anion.

Examples of an onium salt that affords a carboxylic acid through exposure include:
(3) a compound in which one or more fluorine atoms or fluorinated hydrocarbon groups are bonded to a carbon atom adjacent to a carboxylic acid anion, and
(4) a compound in which neither fluorine atom nor fluorinated hydrocarbon group is bonded to a carbon atom adjacent to a carboxylic acid anion.

Among them, as the radiation-sensitive acid generator or the radiation-sensitive acid generating resin, those corresponding to the above (1) are preferable. As the acid diffusion controlling agent, those corresponding to the above (2), (3), or (4) are preferable, and those corresponding to the above (2) or (4) are particularly preferable.

### <Radiation-Sensitive Acid Generator>

The radiation-sensitive acid generator contains an organic acid anion moiety and an onium cation moiety. The radiation-sensitive acid generator is preferably represented by the following formula (pd-1) or (pd-2).

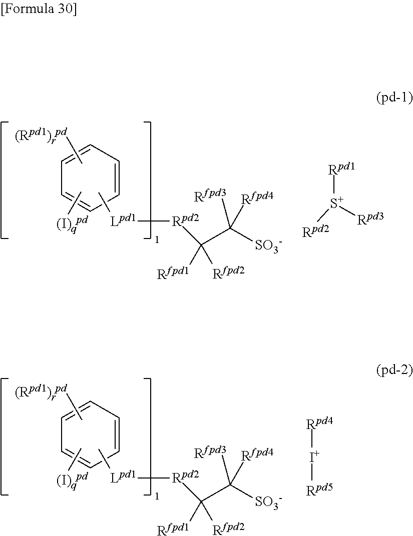

In the formulas (pd-1) and (pd-2), L^{pd1} is a single bond, an ether linkage, an ester linkage, or an alkylene group having 1 to 6 carbon atoms optionally containing an ether linkage or an ester linkage. The alkylene group may be linear, branched, or cyclic.

R^{pd1} is a hydroxy group, a carboxy group, a fluorine atom, a chlorine atom, a bromine atom, an amino group, an alkyl group having 1 to 20 carbon atoms, an alkoxy group having 1 to 20 carbon atoms, an alkoxycarbonyl group having 2 to 10 carbon atoms, an acyloxy group having 2 to 20 carbon atoms, an alkylsulfonyloxy group having 1 to 20 carbon atoms, —NR^{pd6}—C(═O)—R^{pd7}, or —NR^{pd6}—C(═O)—O—R^{pd7}. The alkyl group having 1 to 20 carbon atoms, the alkoxy group having 1 to 20 carbon atoms, or the fluorine atom may be substituted with or replaced by a chlorine atom, a bromine atom, a hydroxy group, an amino group, or an alkoxy group having 1 to 10 carbon atoms. R^{pd6} is a hydrogen atom or an alkyl group having 1 to 6 carbon atoms and optionally containing a halogen atom, a hydroxy group, an alkoxy group having 1 to 6 carbon atoms, an acyl group having 2 to 6 carbon atoms or an acyloxy group having 2 to 6 carbon atoms. R^{pd7} is an alkyl group having 1 to 16 carbon atoms, an alkenyl group having 2 to 16 carbon atoms, or an aryl group having 6 to 12 carbon atoms, and these groups may contain a halogen atom, a hydroxy group, an alkoxy group having 1 to 6 carbon atoms, an acyl group having 2 to 6 carbon atoms, or an acyloxy group having 2 to 6 carbon atoms. The alkyl group, alkoxy group, alkoxycarbonyl group, acyloxy group, acyl group, and alkenyl group may be linear, branched, or cyclic.

Among them, a hydroxy group, —NR^{pd6}—C(═O)—R^{pd7}, a fluorine atom, a chlorine atom, a bromine atom, a methyl group, and a methoxy group are preferable as R^{pd1}.

p^{pd} is an integer satisfying 0≤p^{pd}≤3. R^{pd2} is a hydrogen atom or a monovalent organic group having 1 to 20 carbon atoms when p^{pd} is 0. When p^{pd} is 1, R^{pd2} is a single bond or a divalent linking group having 1 to 20 carbon atoms, and when p^{pd} is 2 or 3, R^{pd2} is a trivalent or tetravalent linking group having 1 to 20 carbon atoms and the linking group may contain an oxygen atom, a sulfur atom, or a nitrogen atom. Examples of the monovalent organic group in the case in which p is 0 include the same groups as those disclosed above as examples of the hydrocarbon group constituting the fluorinated hydrocarbon group, and monovalent groups in which some of the methylene groups constituting the hydrocarbon group are replaced by an ether group or an ester group.

R^{fpd1} to R^{fpd4} are each independently a hydrogen atom, a fluorine atom, or a trifluoromethyl group, and at least one of them is a fluorine atom or a trifluoromethyl group. In particular, it is preferable that both R^{fpd3} and R^{fpd4} are fluorine atoms.

R^{pd1}, R^{pd2}, R^{pd3}, R^{pd4} and R^{pd5} are each independently a monovalent hydrocarbon group having 1 to 20 carbon atoms and optionally containing a heteroatom. R^{pd1}, R^{pd2}, and R^{pd3} may contain one or more fluorine atoms, and R^{pd4} and R^{pd5} may contain one or more fluorine atoms. Any two of R^{pd1}, R^{pd2}, and R^{pd3} may be bonded to each other to form a ring together with the sulfur atom to which they are bonded. The monovalent hydrocarbon group may be linear, branched, or cyclic, and examples thereof include the same groups as those disclosed as examples of the hydrocarbon group constituting the fluorinated hydrocarbon group. Some or all of the hydrogen atoms of these groups may be replaced by a hydroxy group, a carboxy group, a halogen atom, a cyano group, an amide group, a nitro group, a mercapto group, a sultone group, a sulfone group, or a sulfonium salt-containing group.

q^{pd} and r^{pd} are integers satisfying 0≤q^{pd}≤5, 0≤r^{pd}≤3, and 0≤q^{pd}+r^{pd}≤5. q^{pd} is preferably an integer satisfying 1≤q^{pd}≤3, and more preferably 2 or 3. r^{pd} is preferably an integer satisfying 0≤r^{pd}≤2.

Examples of the organic acid anion moieties of the radiation-sensitive acid generators represented by the formulas (pd-1) and (pd-2) include, but are not limited to, those shown below. While all of those shown below are organic acid anion moieties having an iodine-substituted aromatic ring structure, organic acid anion moieties having no iodine-substituted aromatic ring structure that can be suitably employed include structures in which the iodine atoms in the formulas shown below are replaced by an atom or group other than an iodine atom such as a hydrogen atom or other substituent.

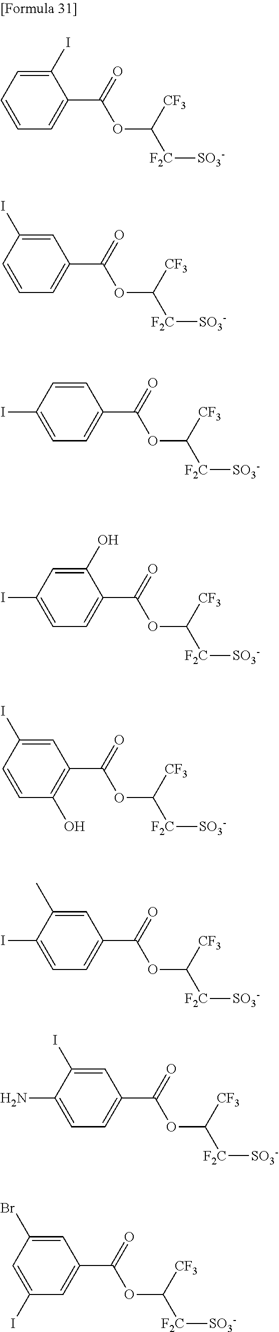

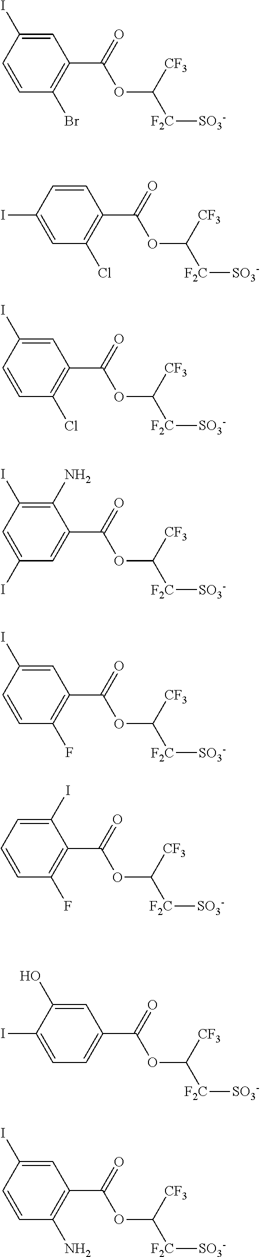

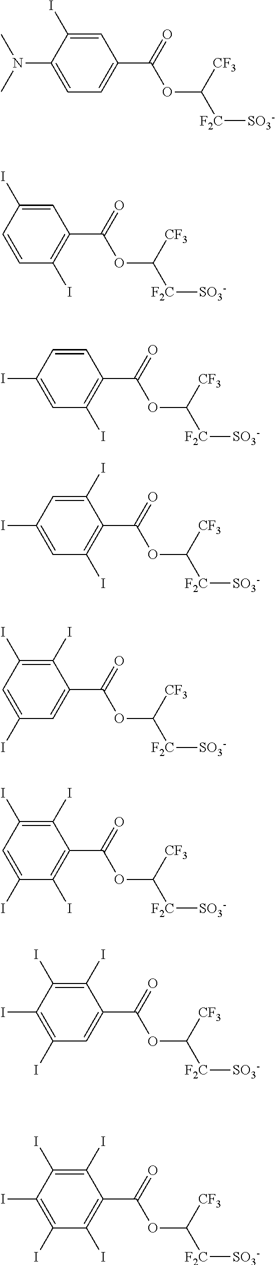

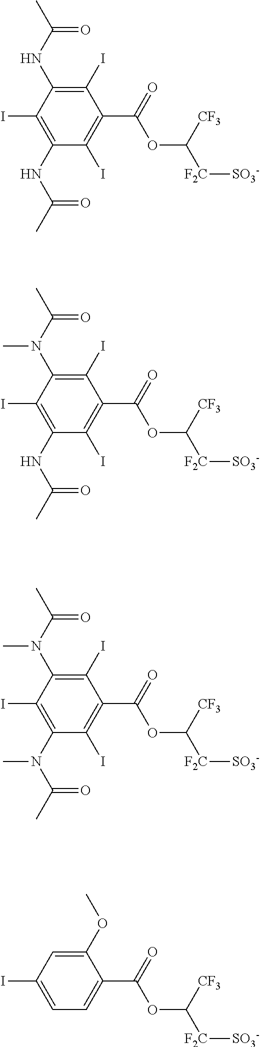

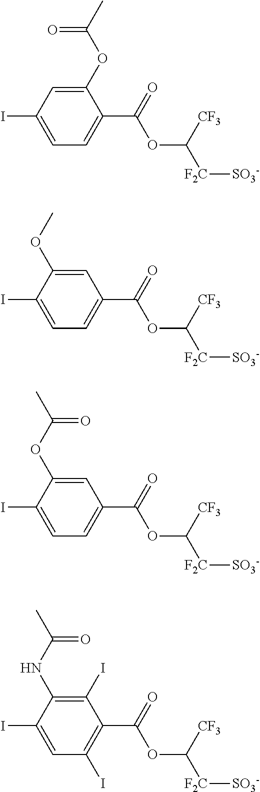

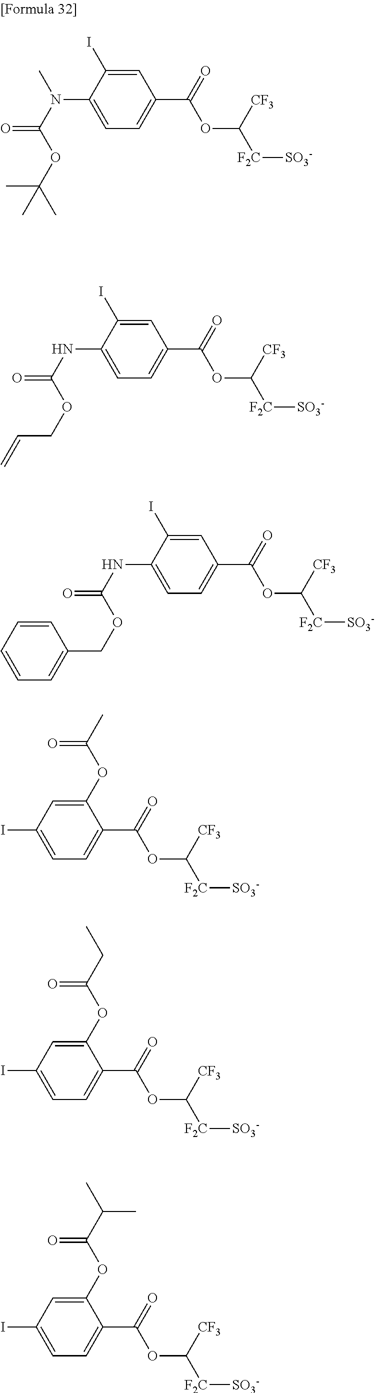

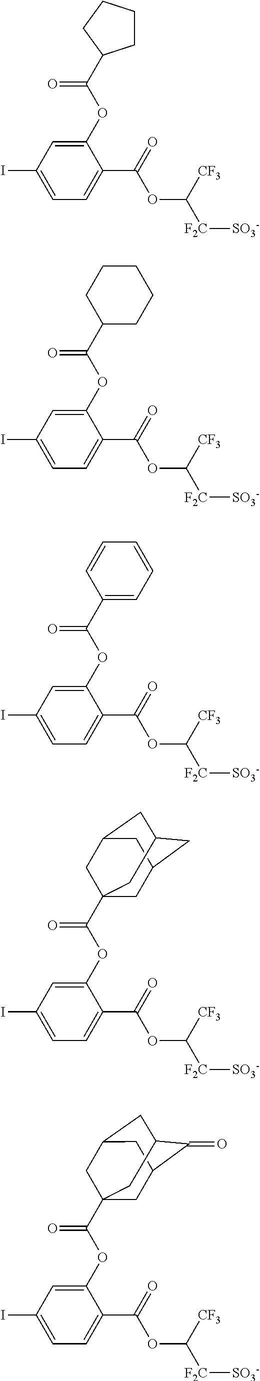

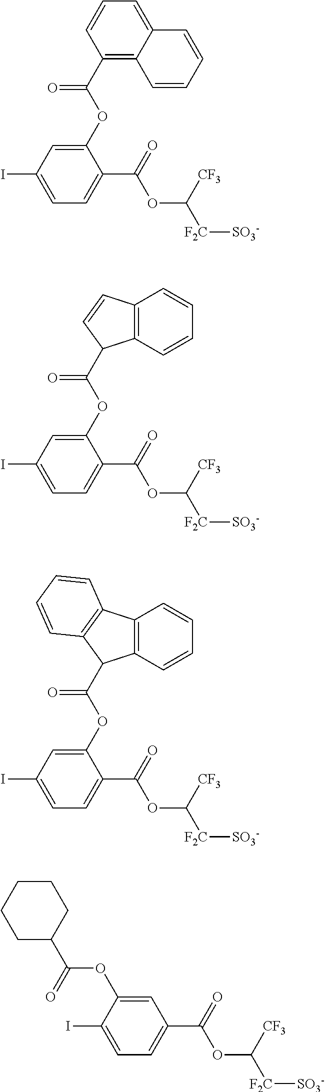

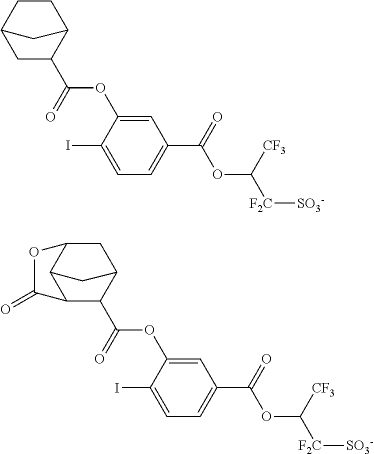

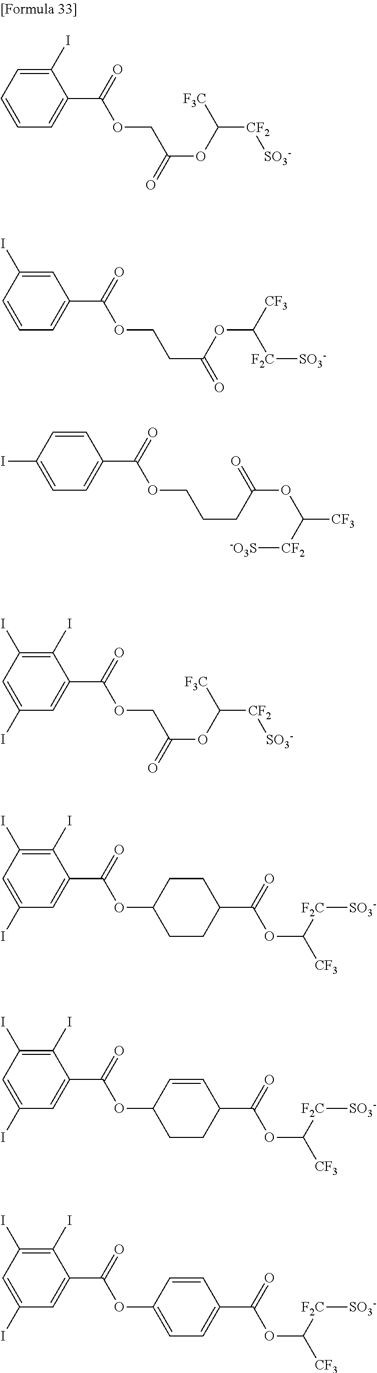

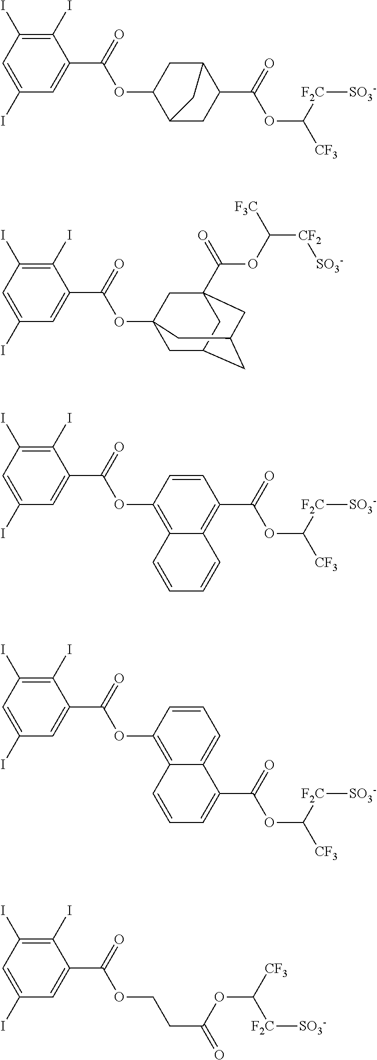

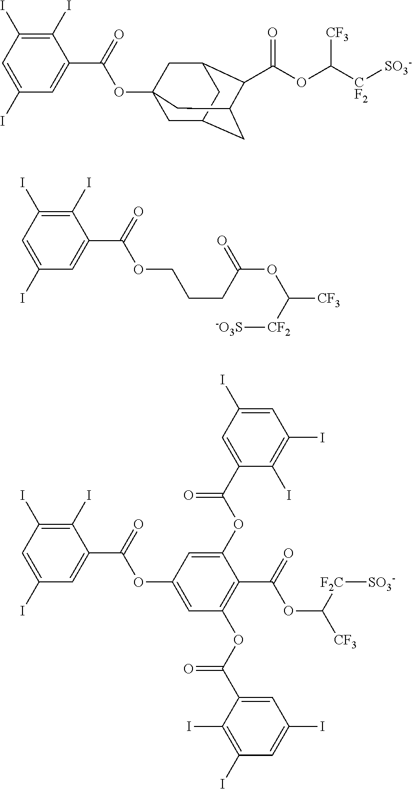

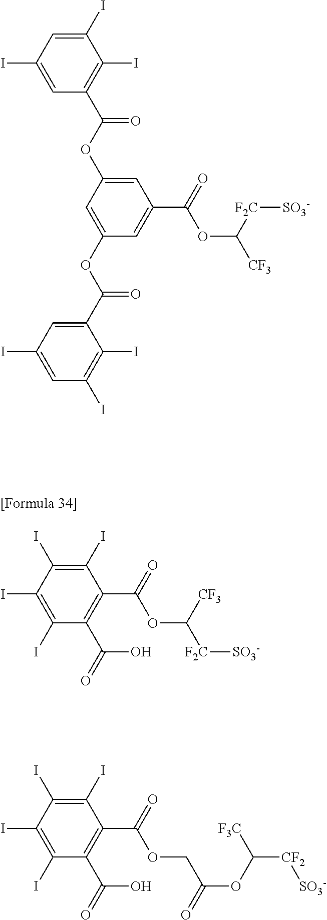

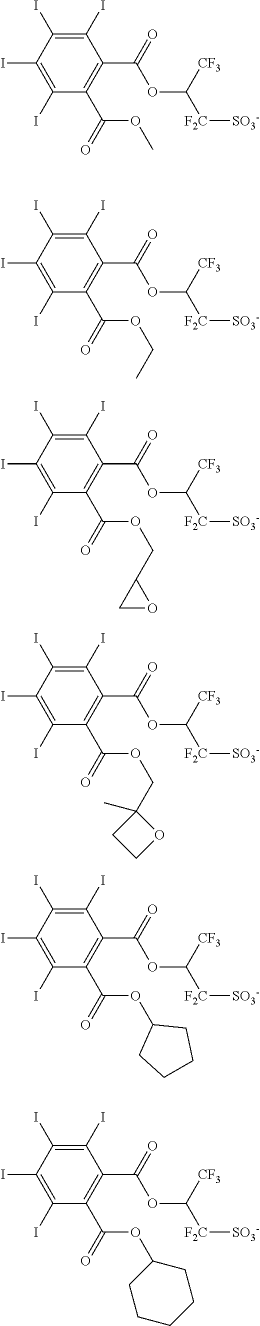

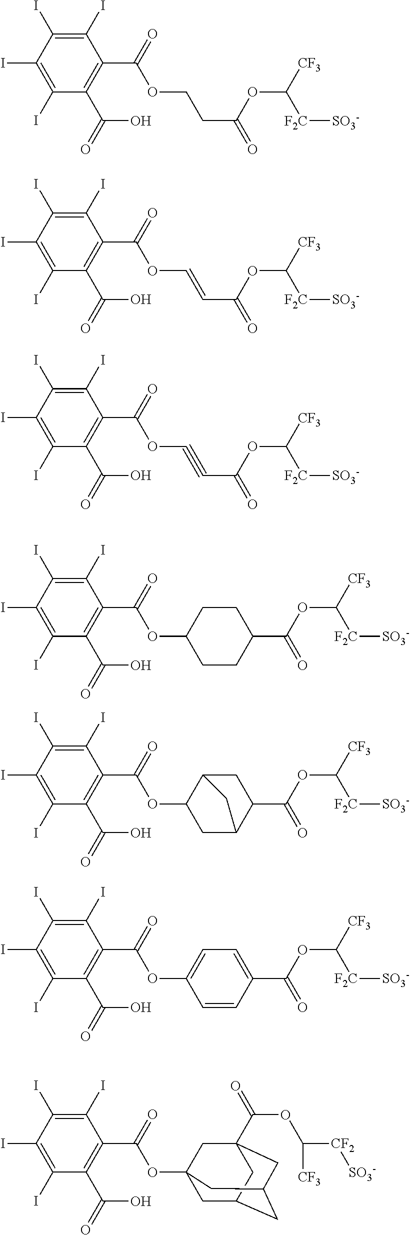

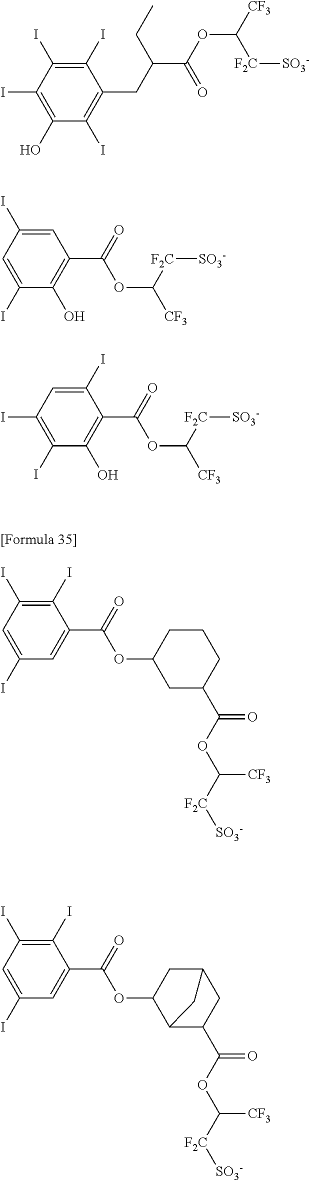

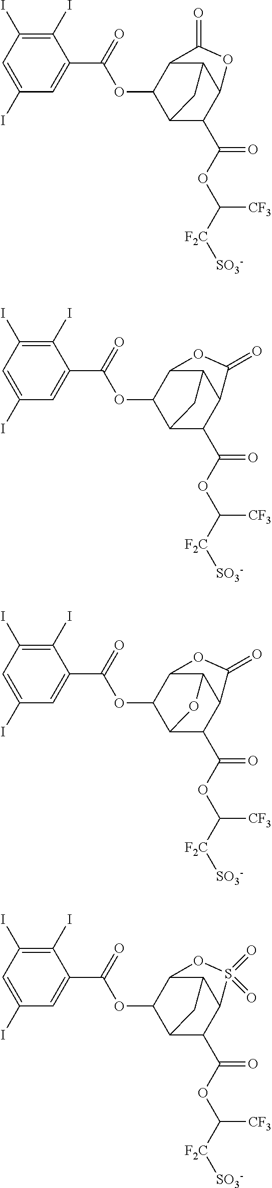

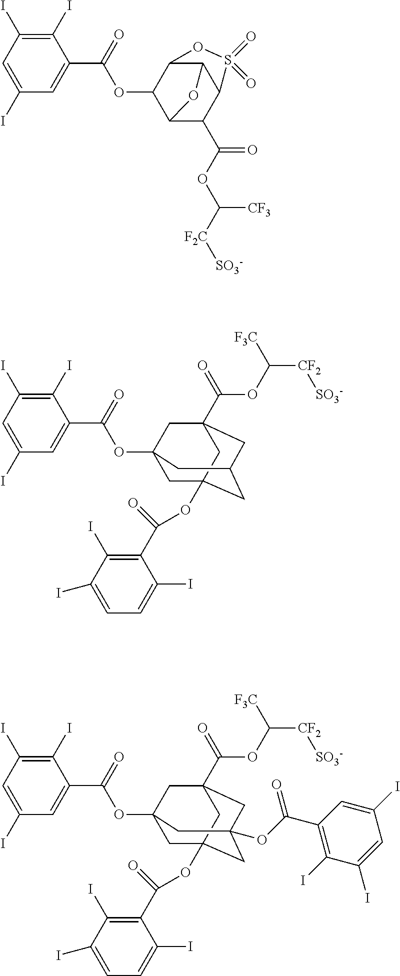

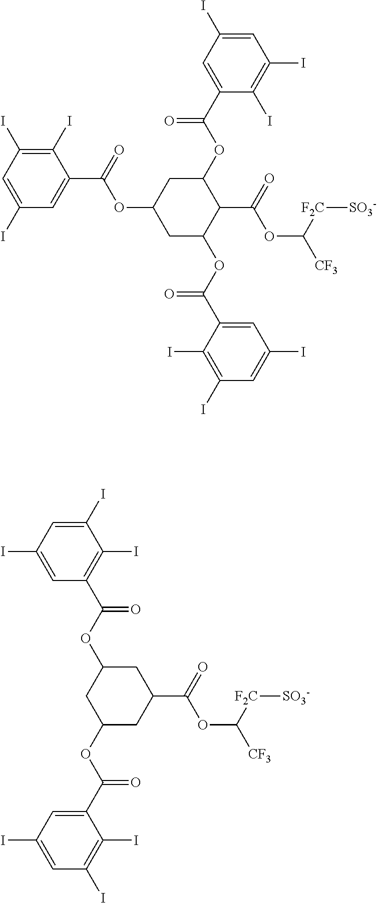

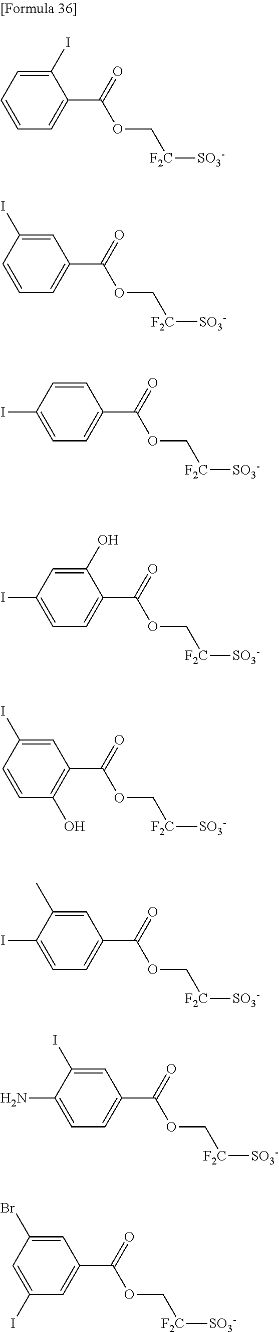

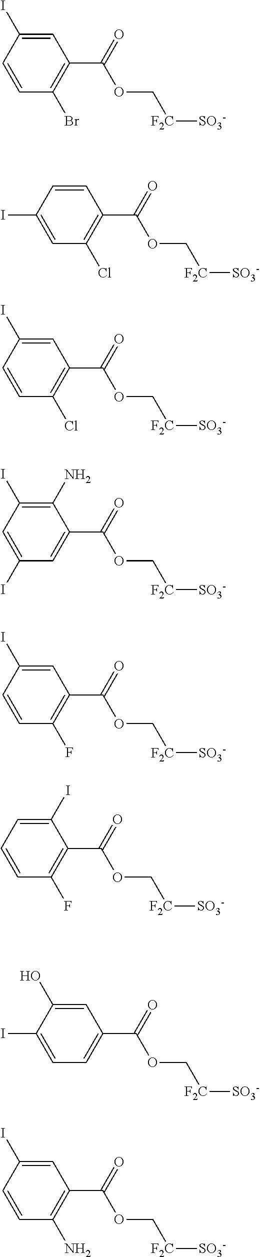

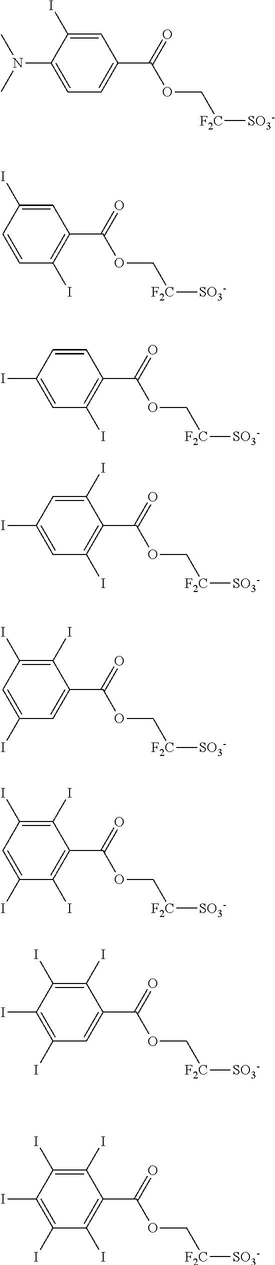

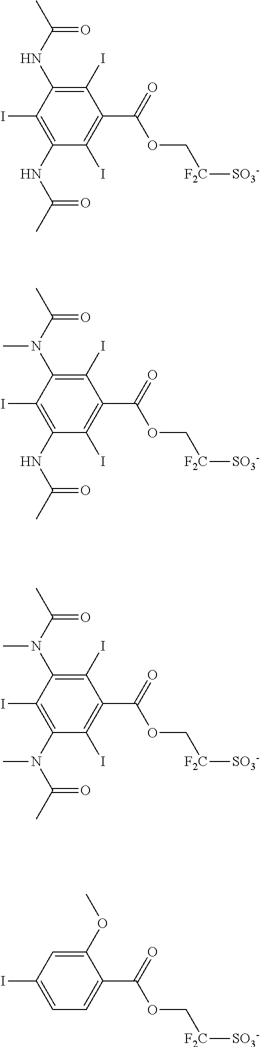

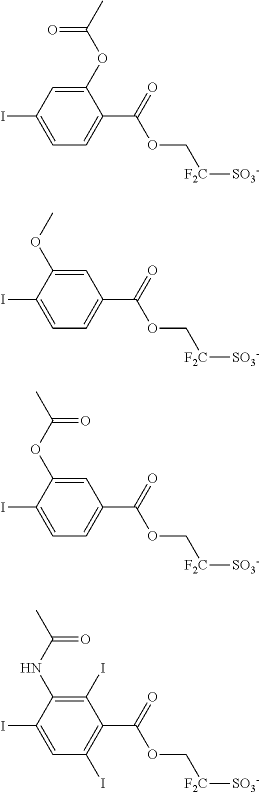

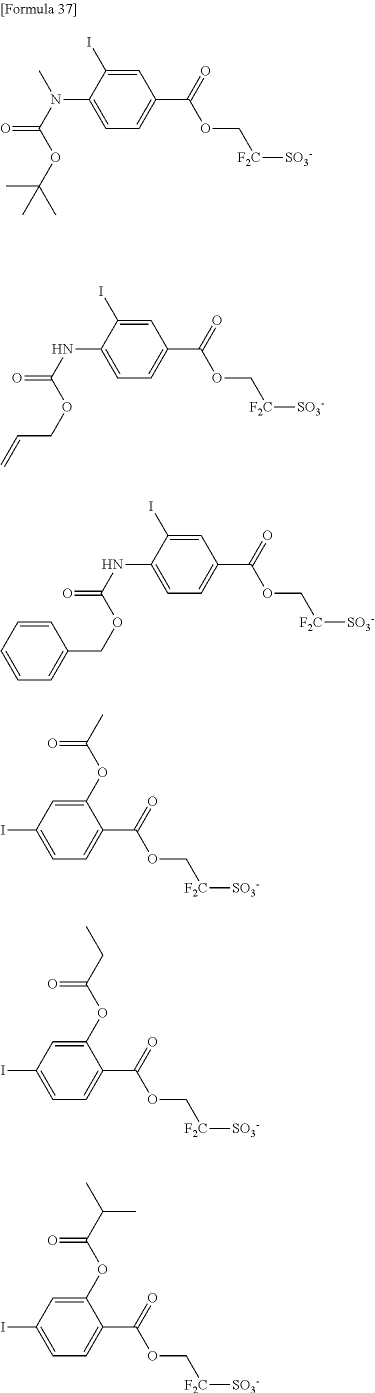

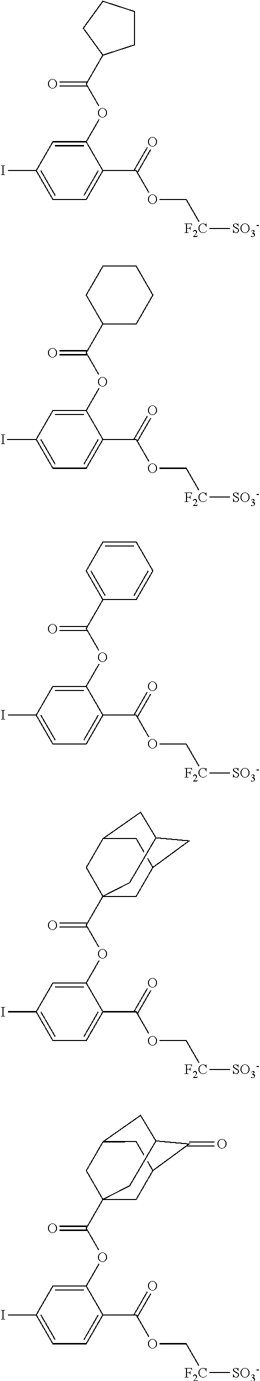

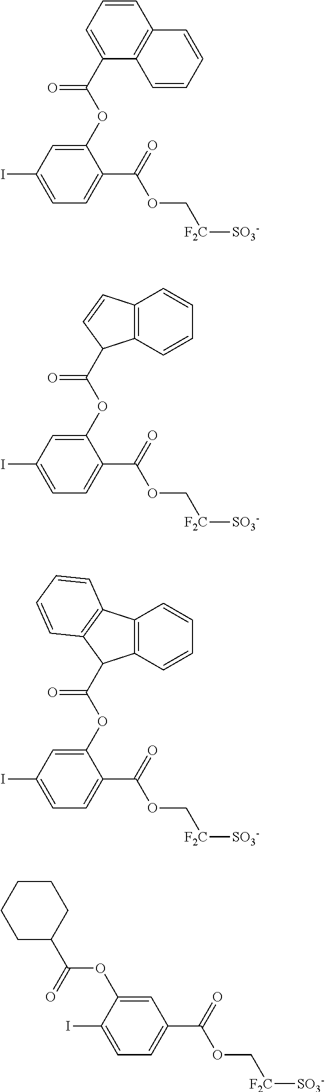

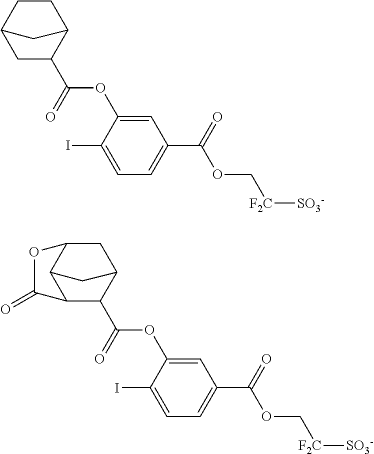

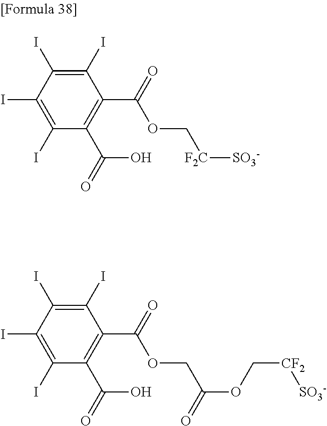

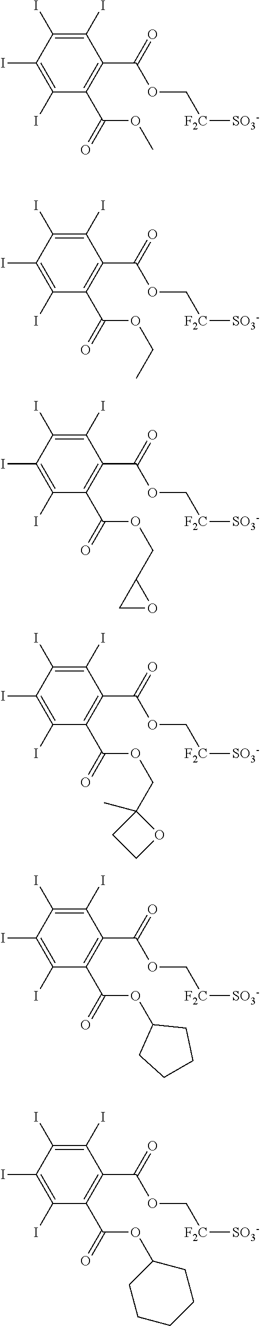

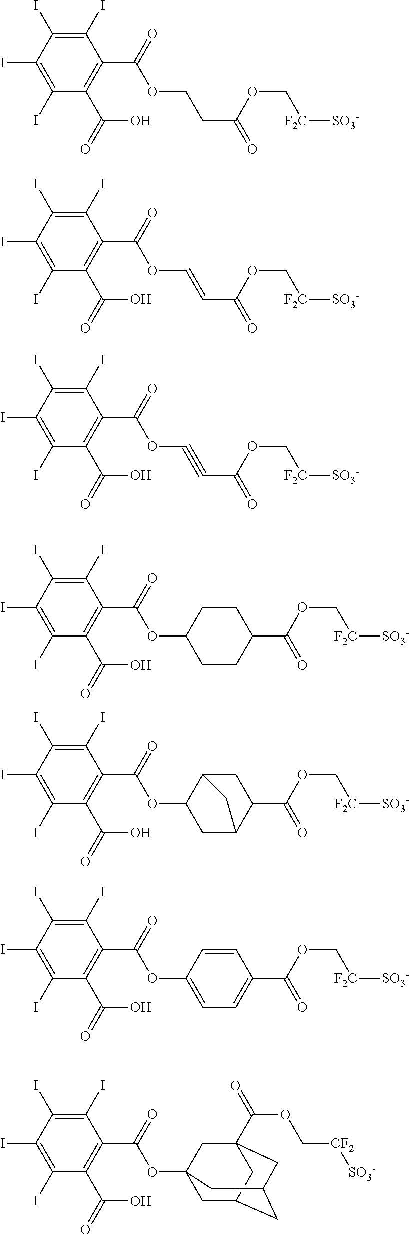

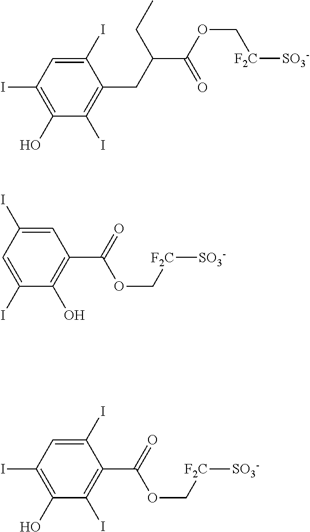

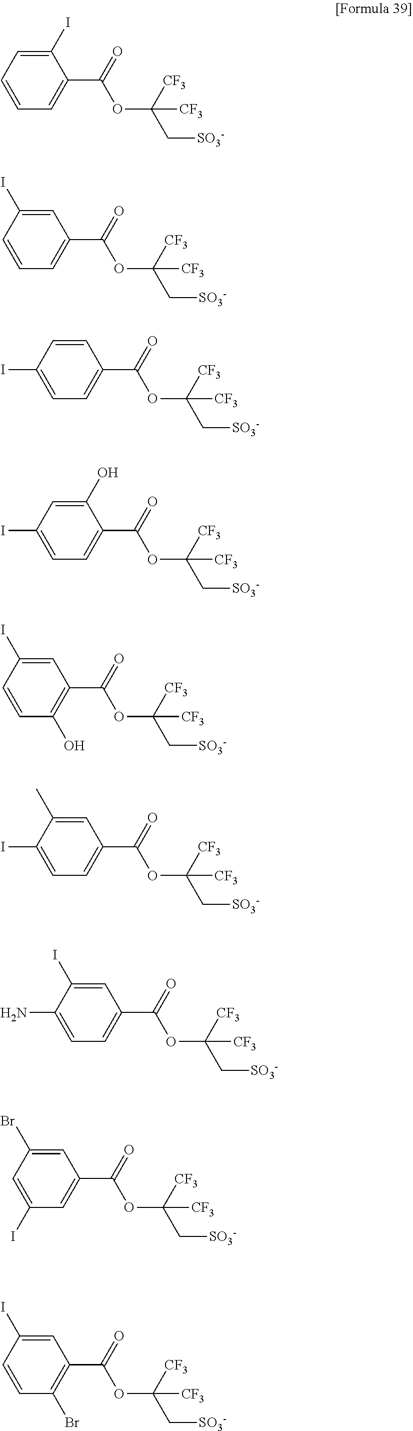

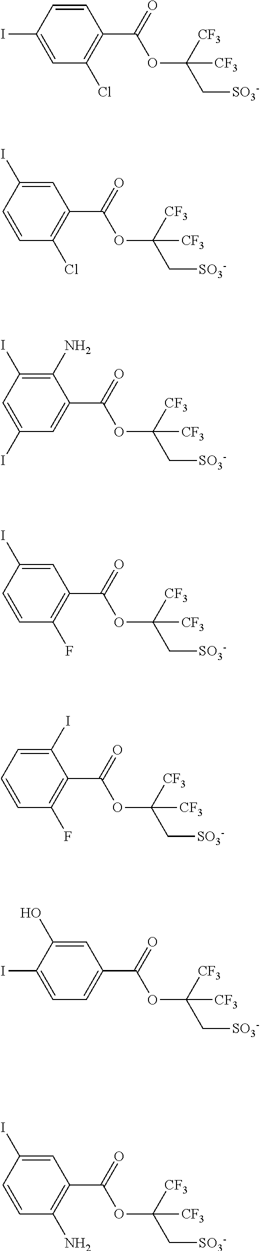

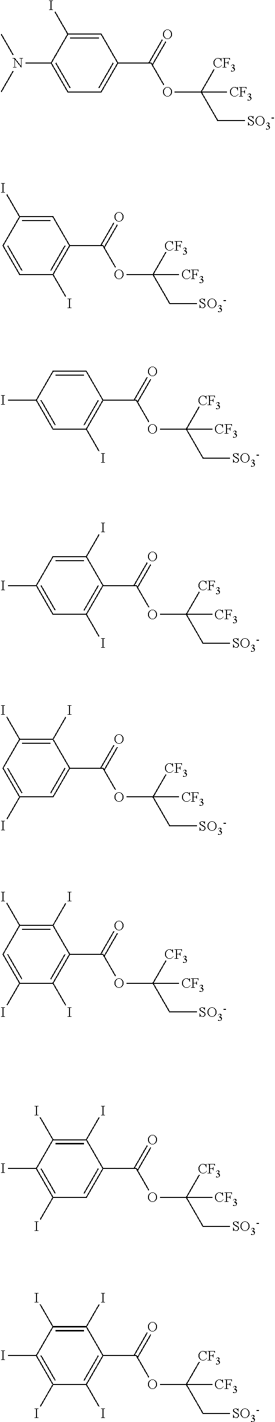

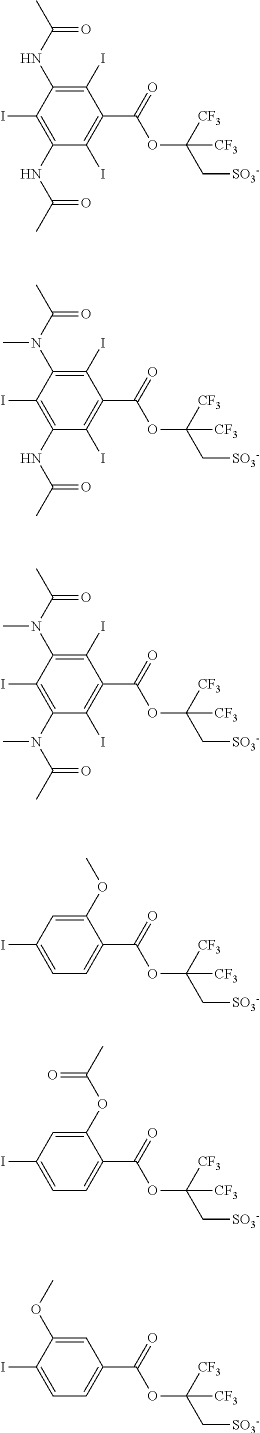

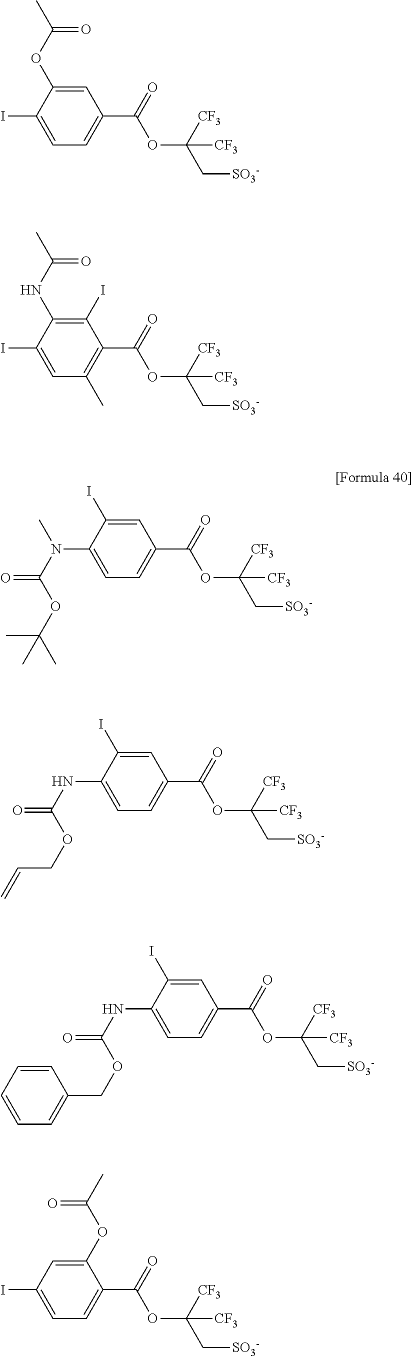

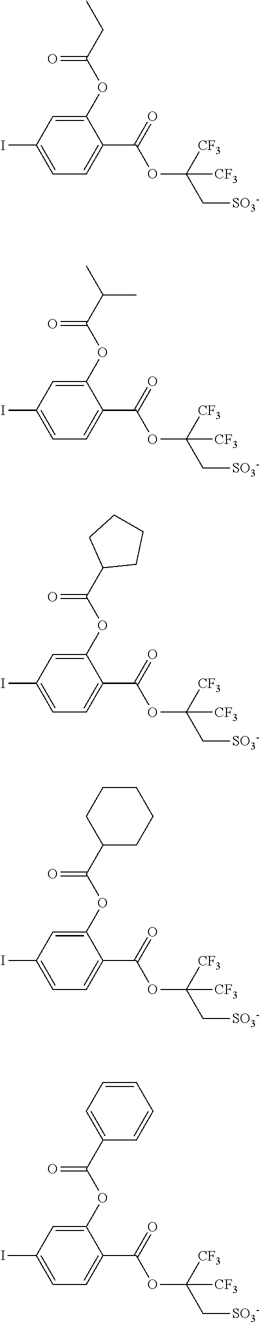

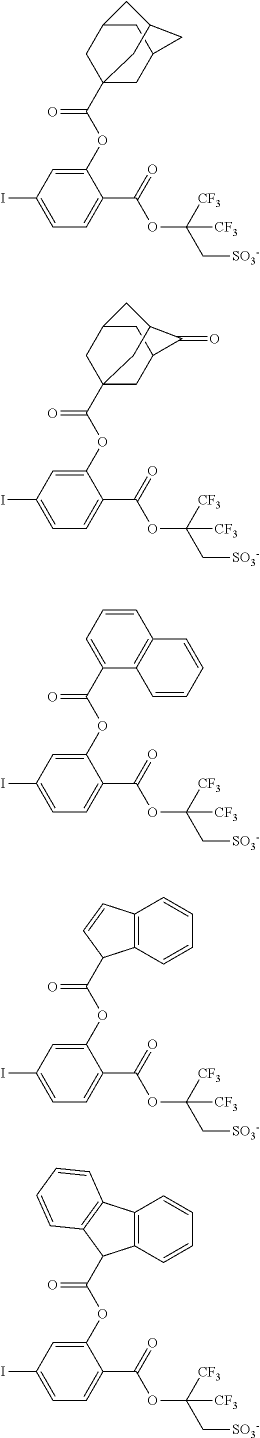

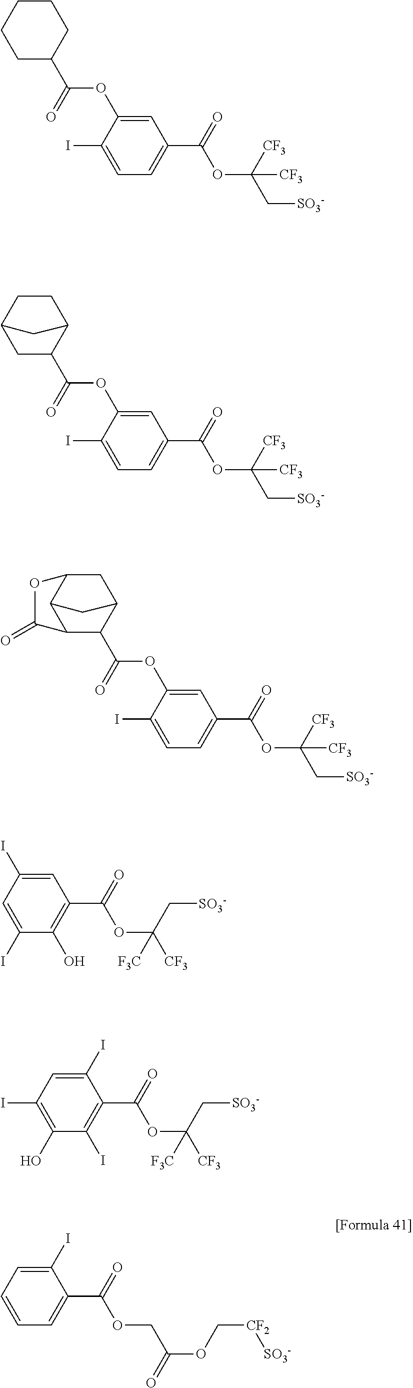

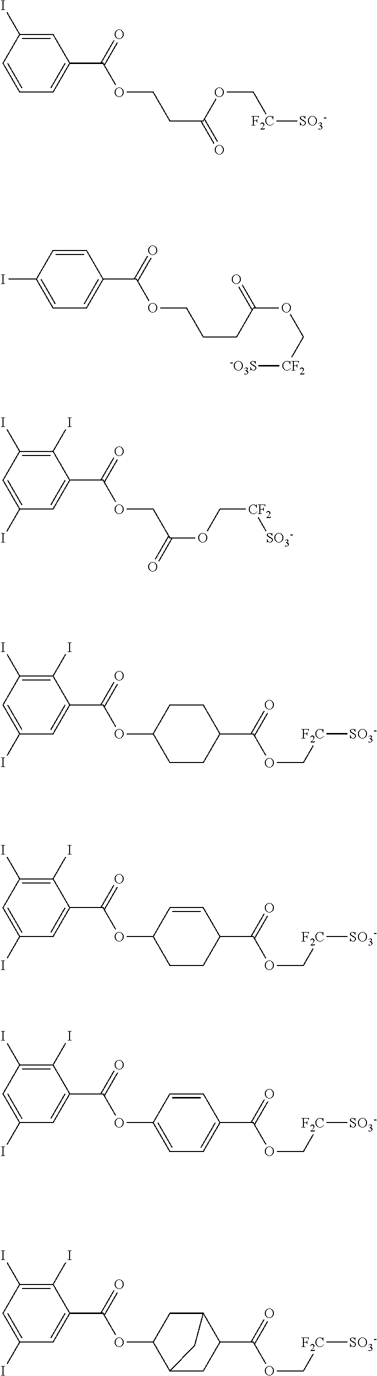

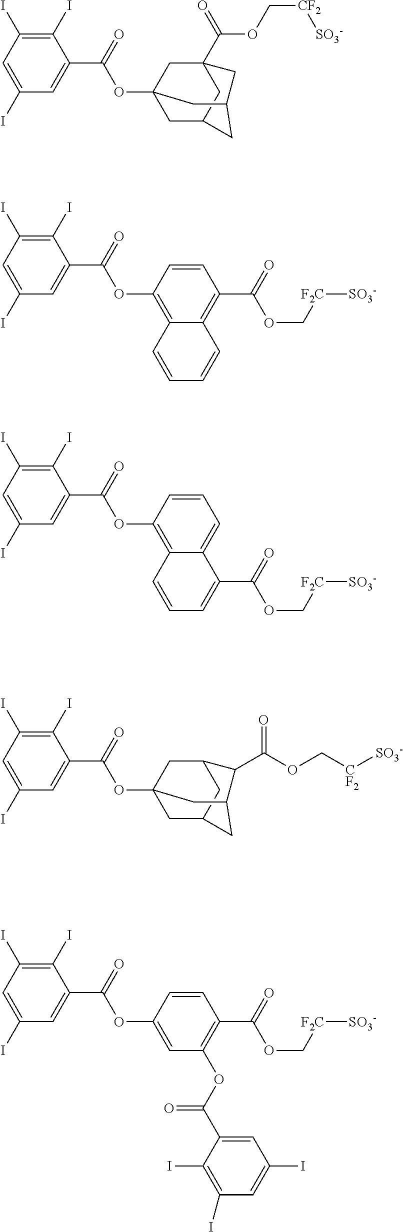

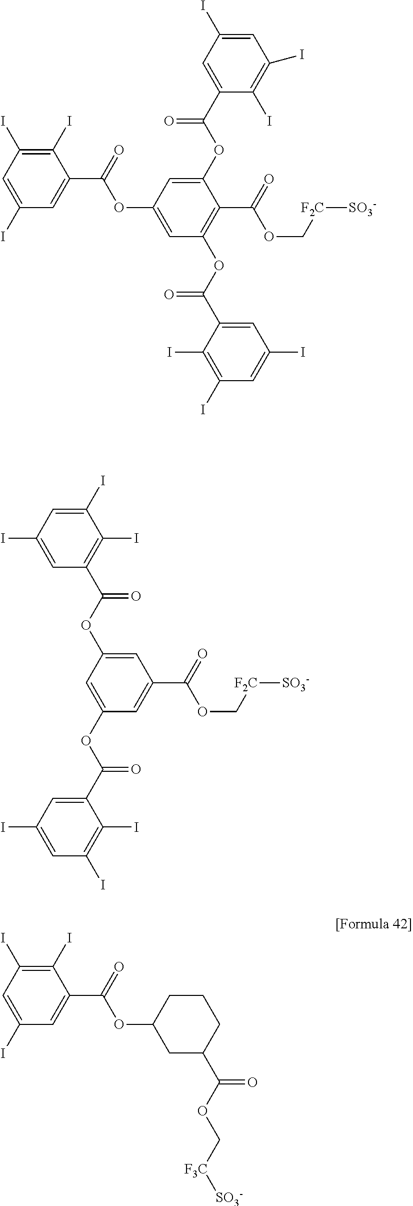

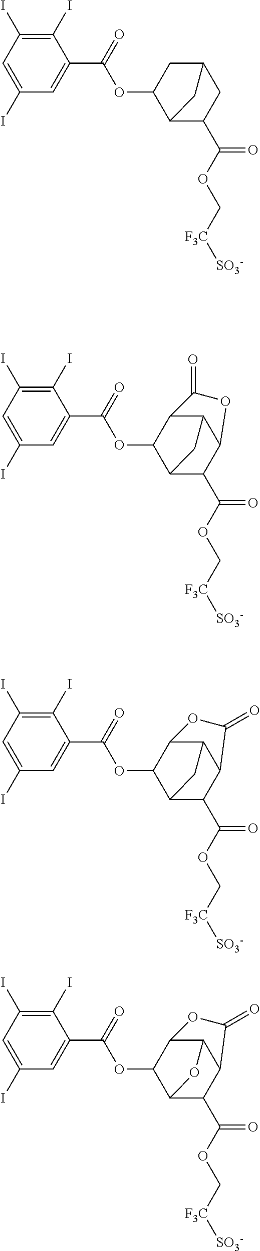

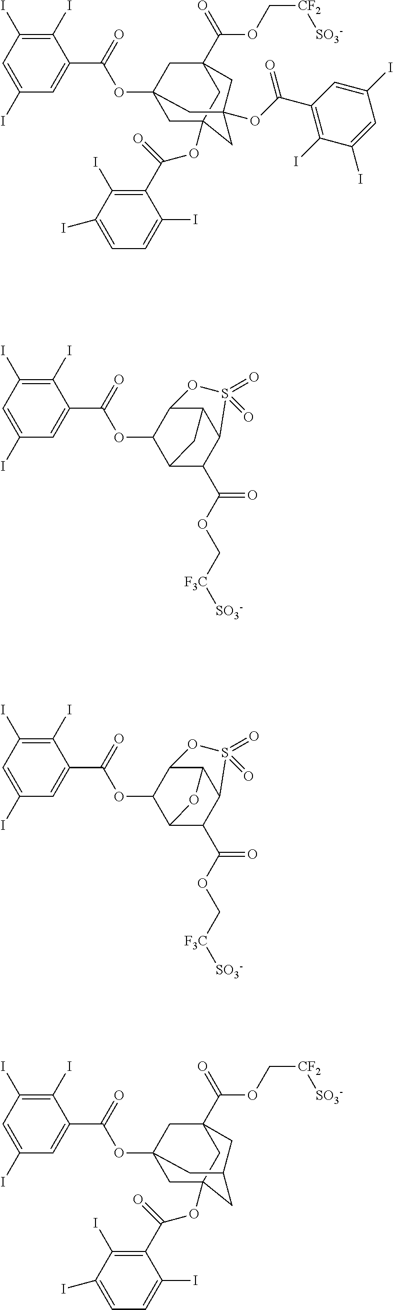

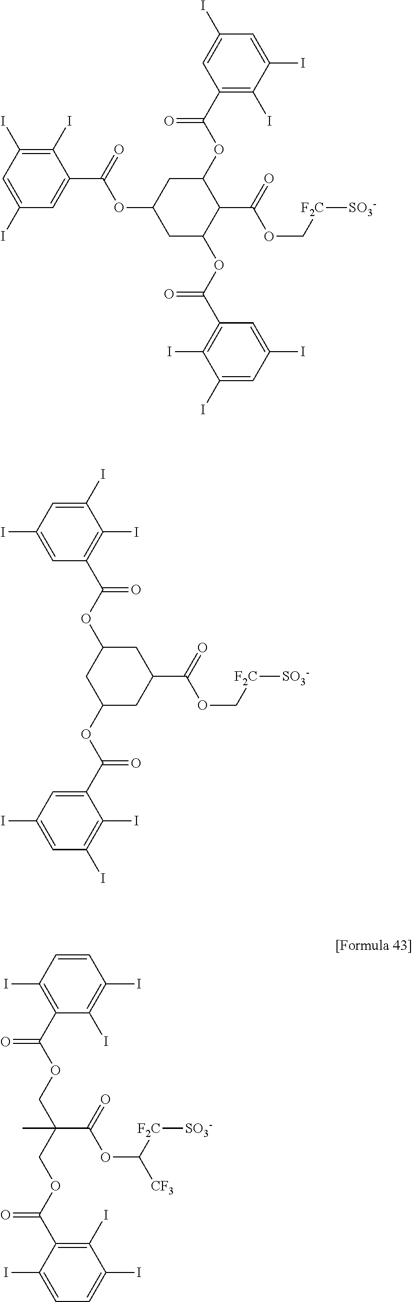

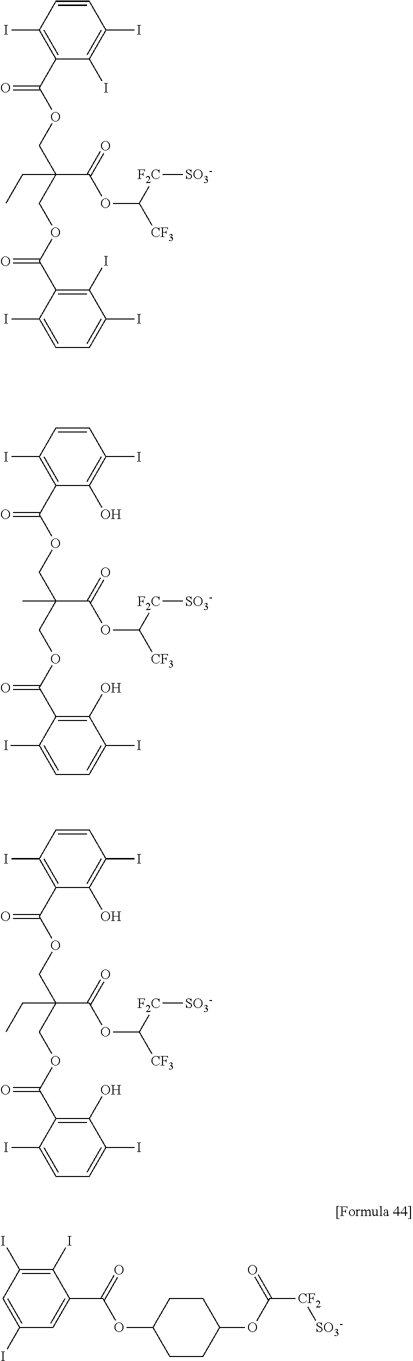

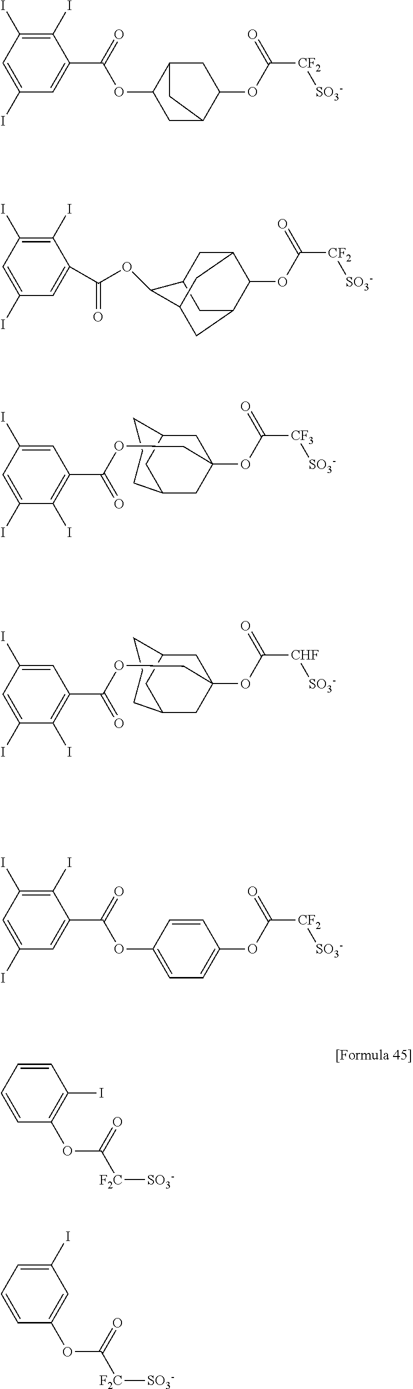

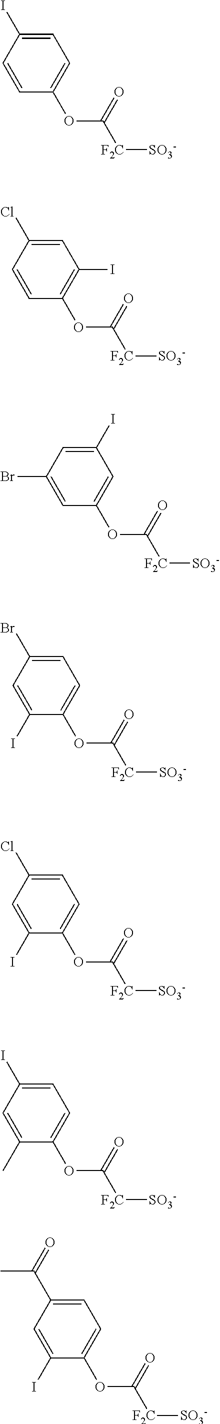

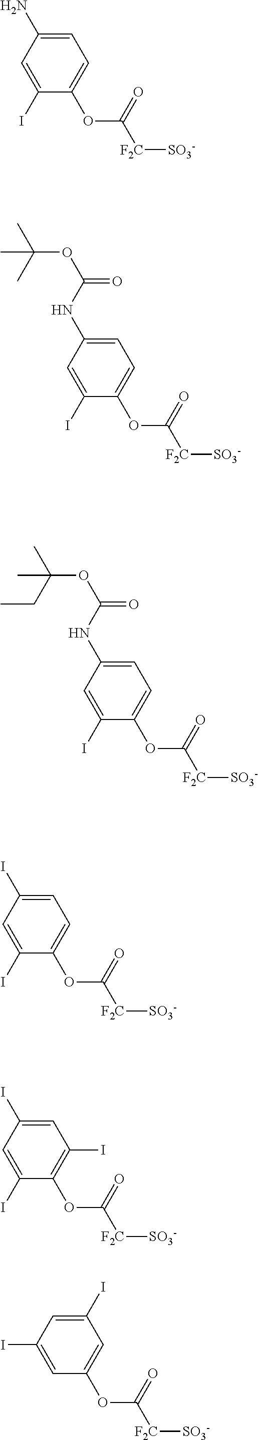

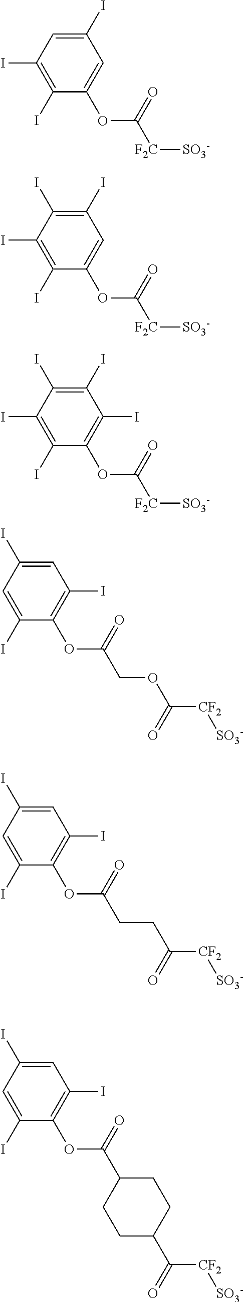

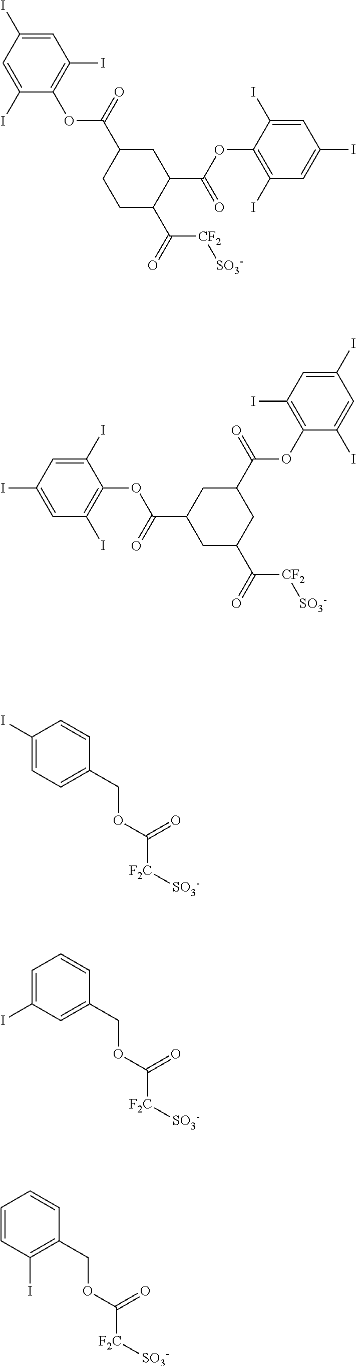

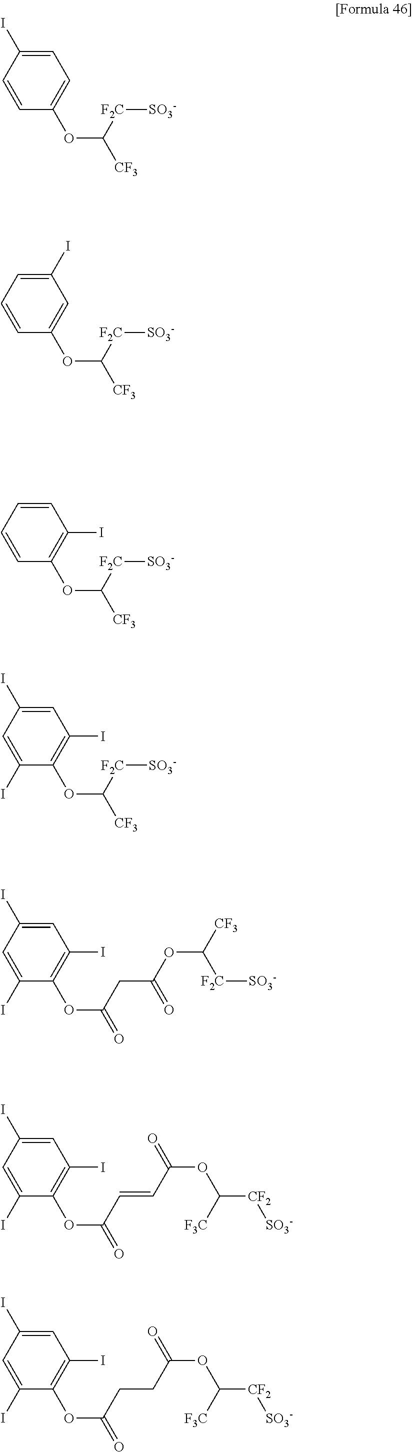

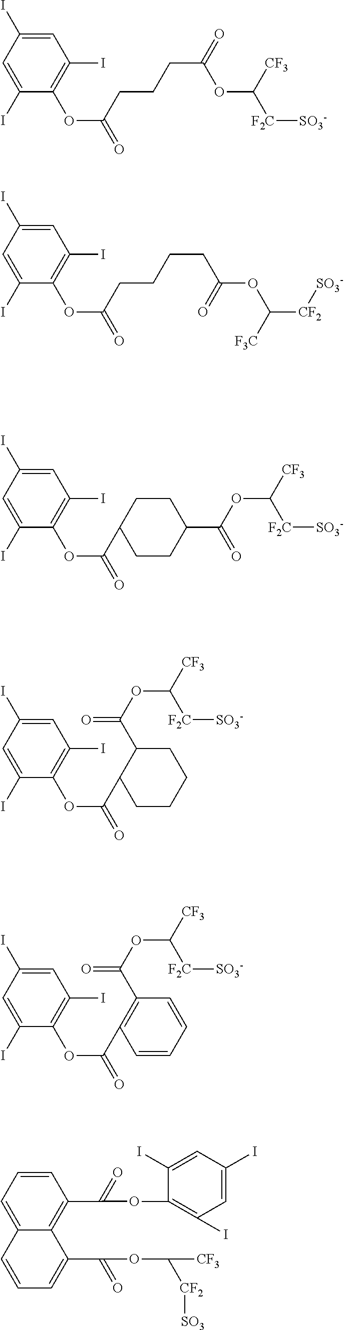

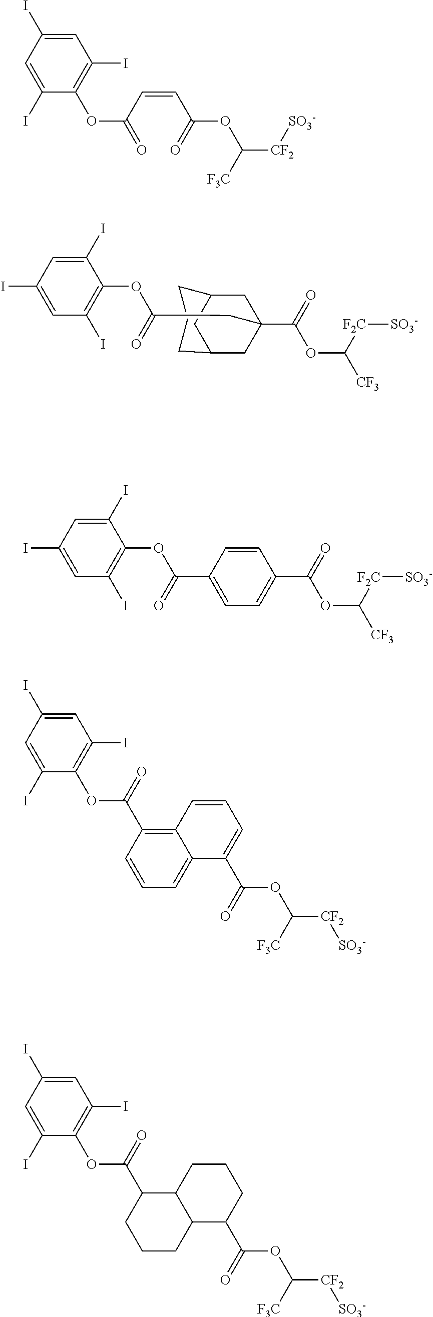

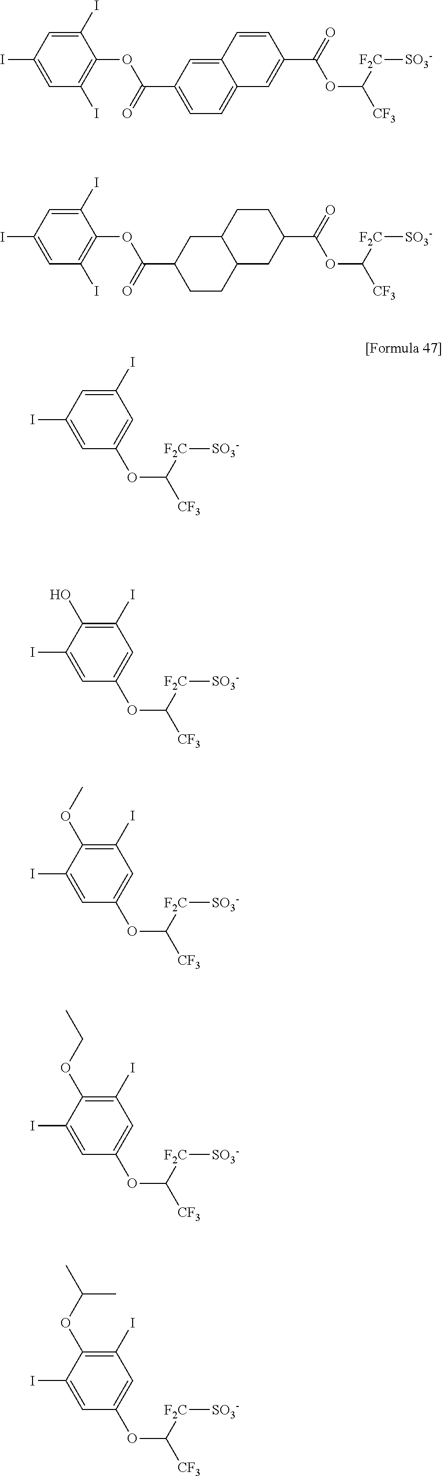

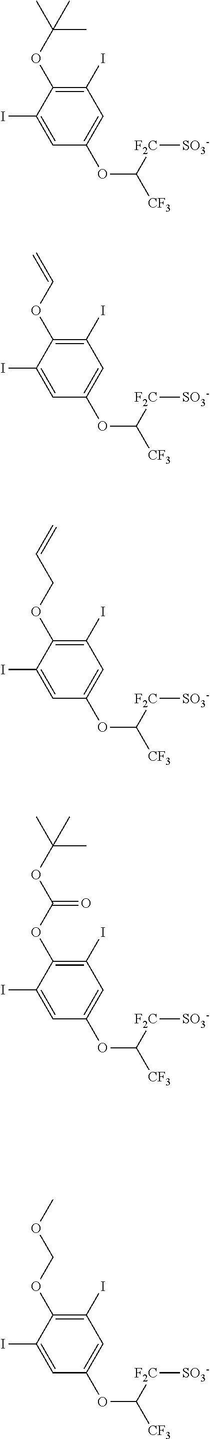

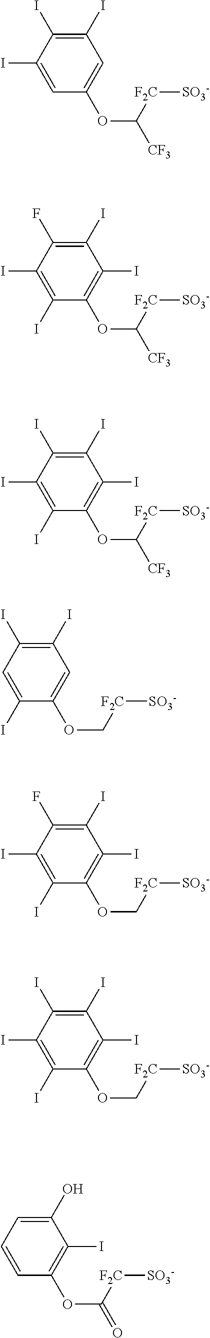

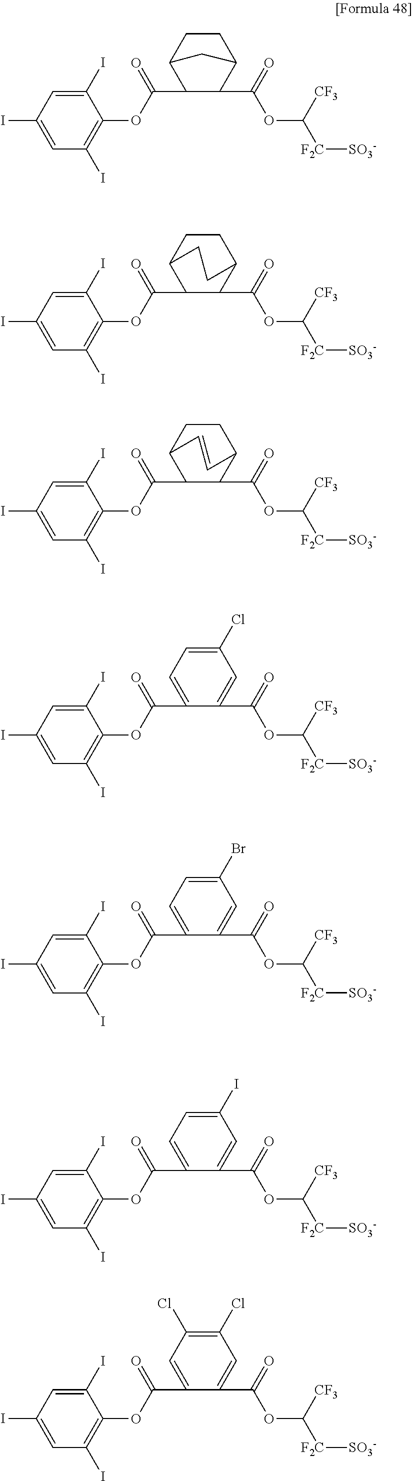

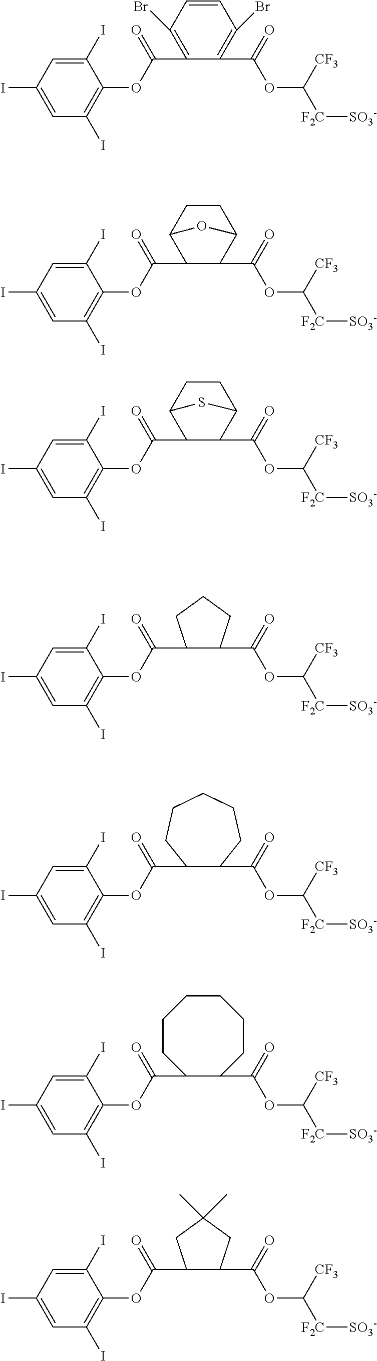

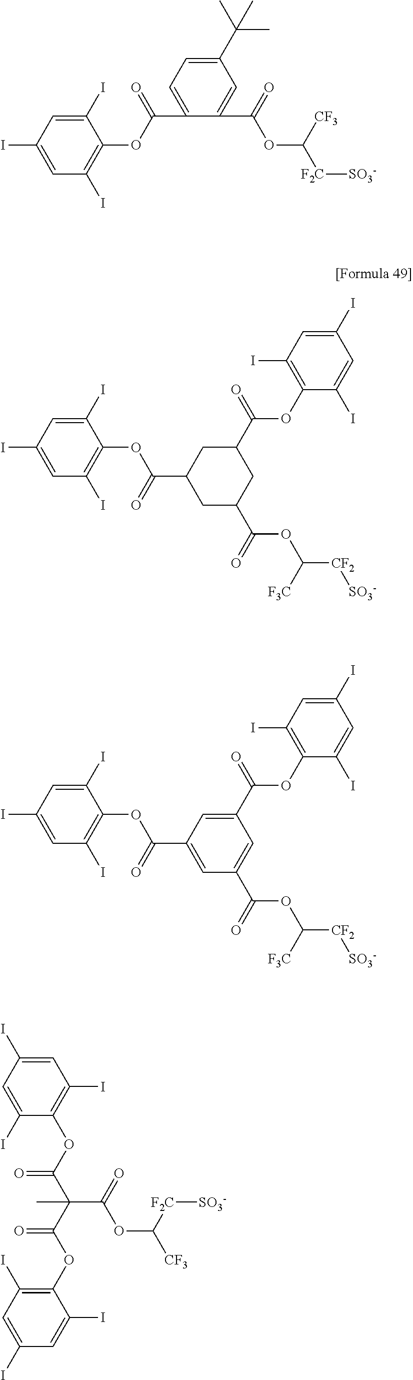

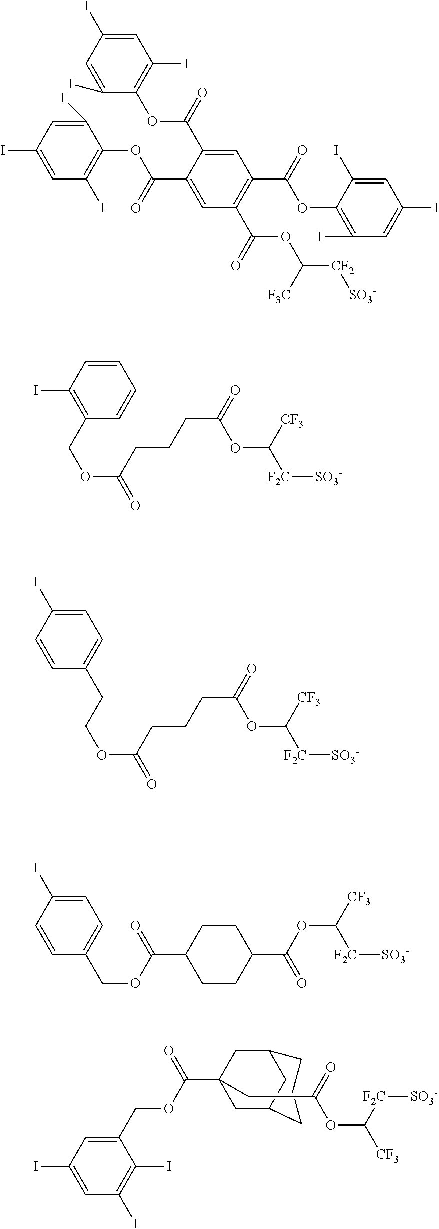

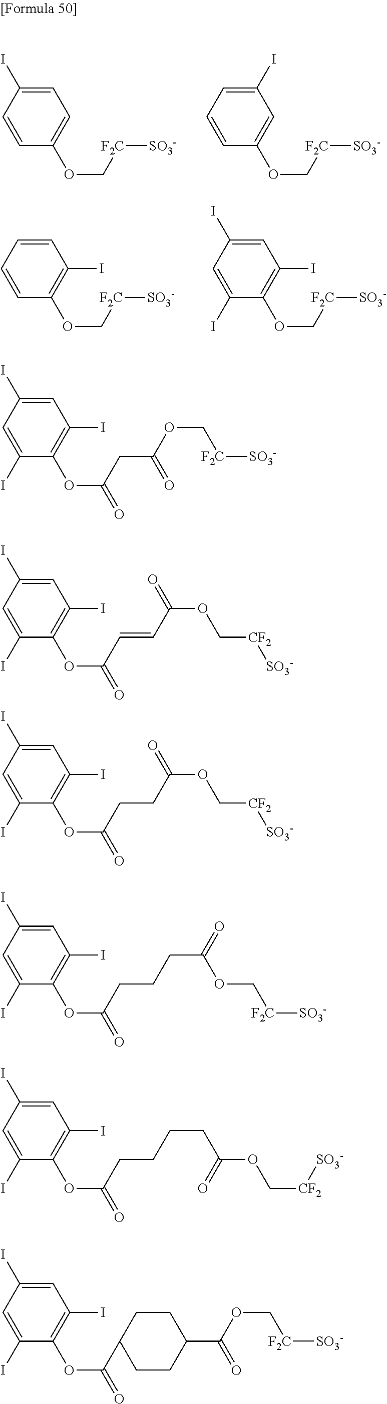

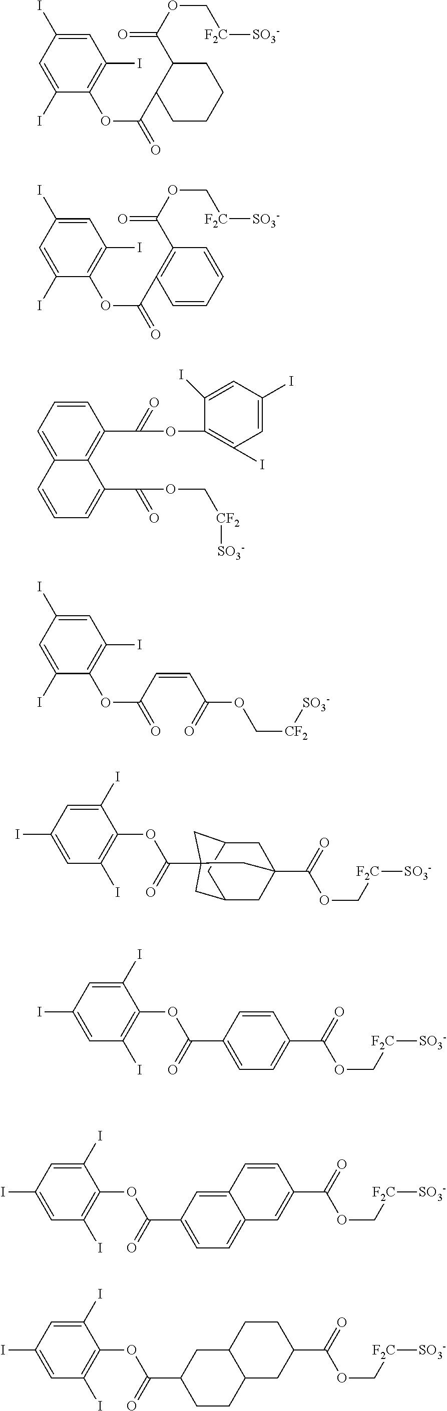

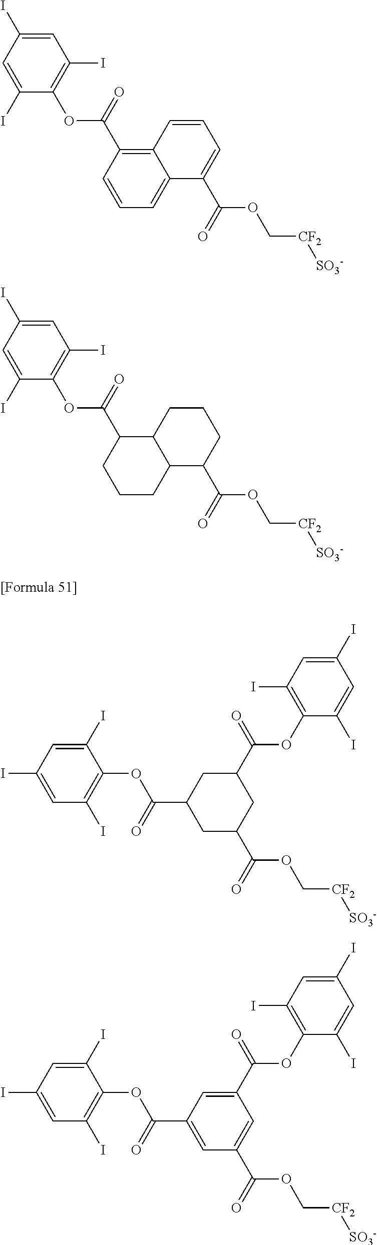

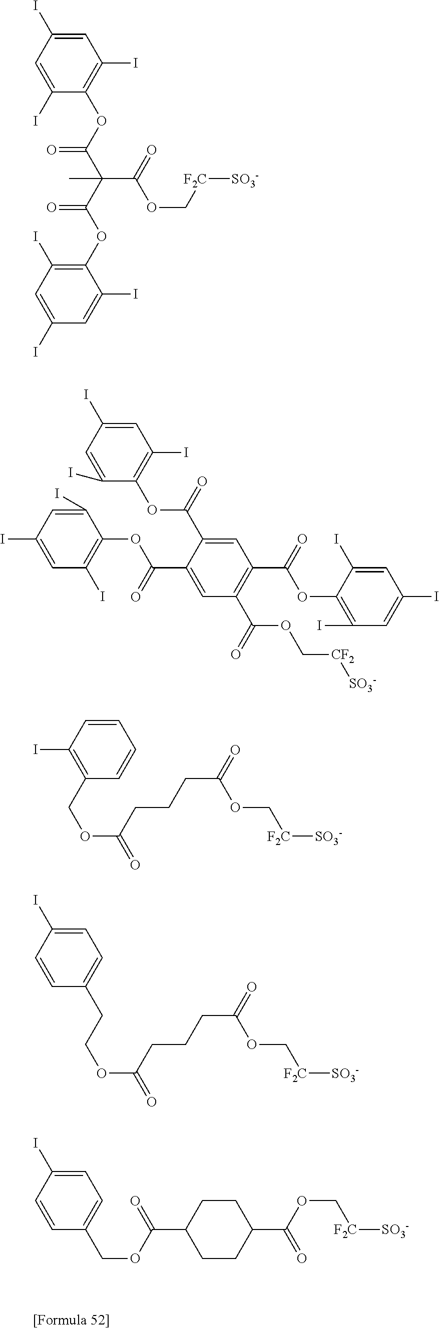

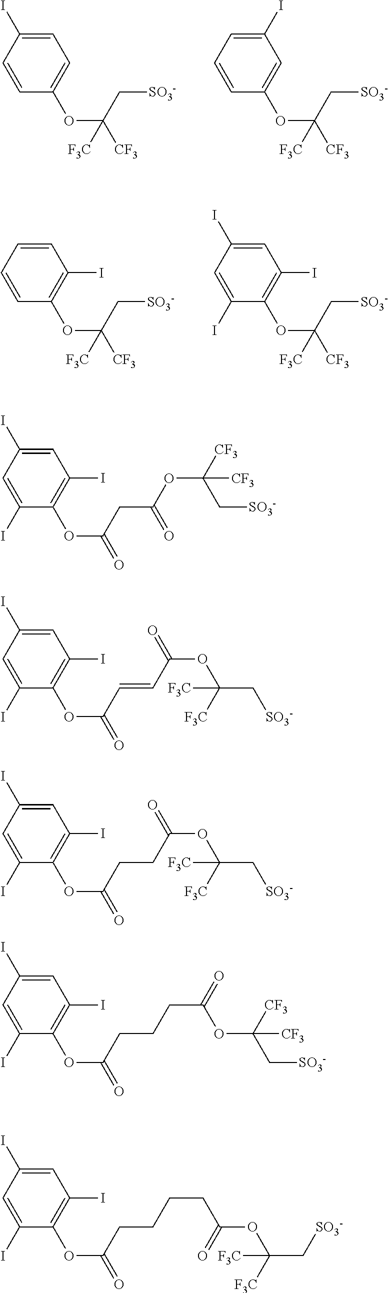

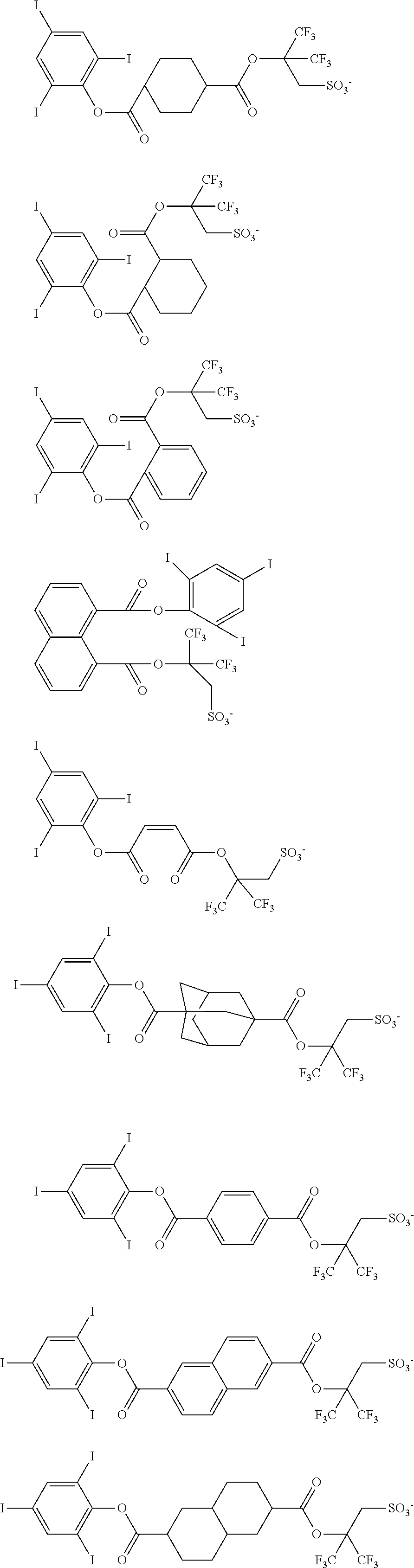

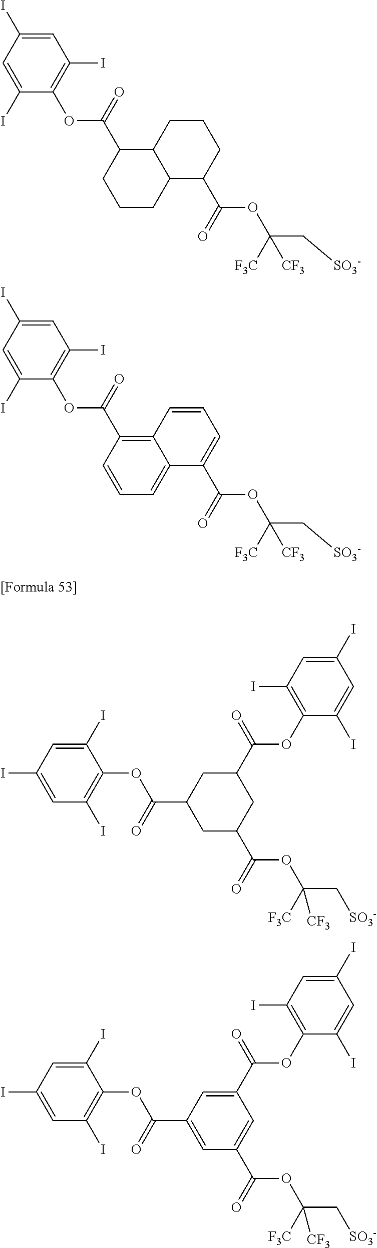

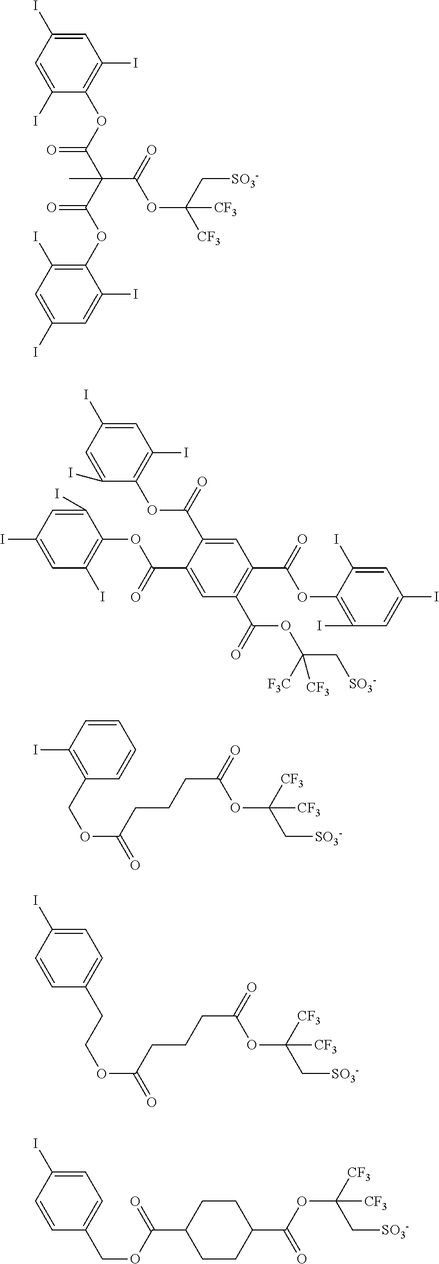

As the onium cation moieties in the radiation-sensitive acid generators represented by the formulas (pd-1) and (pd-2), an onium cation moiety in a structural unit of a radiation-sensitive acid generating resin can be suitably employed.

The radiation-sensitive acid generators represented by the above formulas (pd-1) and (pd-2) can also be synthesized by a known method, particularly by a salt exchange reaction.

These radiation-sensitive acid generators may be used singly or two or more thereof may be used in combination. The content of the radiation-sensitive acid generator is preferably 3 parts by mass or more, more preferably 5 parts by mass or more, and still more preferably 7 parts by mass or more based on 100 parts by mass of the base resin (when a radiation-sensitive acid generating resin and a resin described below are contained, the total amount of them is taken as the basis). The content is preferably 20 parts by mass or less, more preferably 15 parts by mass or less, and still more preferably 13 parts by mass or less based on 100 parts by mass of the resin. This makes it possible to exhibit superior sensitivity or CDU performance when forming a resist pattern.

### <Acid Diffusion Controlling Agent>

The acid diffusion controlling agent contains an organic acid anion moiety and an onium cation moiety and generates an acid having a higher pKa than an acid to be generated from the radiation-sensitive acid generator through irradiation with radiation. The acid diffusion controlling agent is preferably represented by the following formula (S-1) or (S-2).

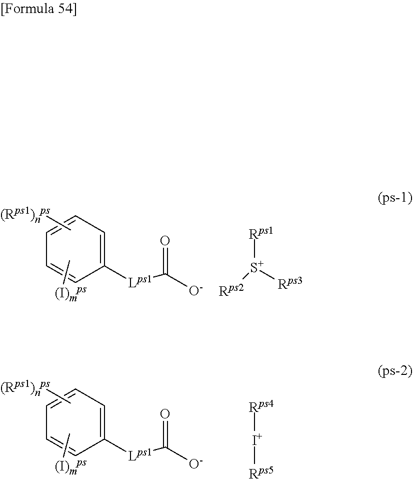

In the formulas (ps-1) and (ps-2), R^{ps1} is a hydrogen atom, a hydroxy group, a fluorine atom, a chlorine atom, an amino group, a nitro group, a cyano group, an alkyl group having 1 to 6 carbon atoms, an alkoxy group having 1 to 6 carbon atoms, an acyloxy group having 2 to 6 carbon atoms, an alkylsulfonyloxy group having 1 to 4 carbon atoms, —NR^{ps1A}—C(═O)—R^{ps1B}, or —NR^{ps1A}—C(═O)—O—R^{ps1B}. The alkyl group having 1 to 6 carbon atoms, the alkoxy group having 1 to 6 carbon atoms, the acyloxy group having 2 to 6 carbon atoms, and the alkylsulfonyloxy group having 1 to 4 carbon atoms may be substituted with a halogen atom. R^{ps1A} is a hydrogen atom or an alkyl group having 1 to 6 carbon atoms, and R^{ps1B} is an alkyl group having 1 to 6 carbon atoms or an alkenyl group having 2 to 8 carbon atoms.

The alkyl group having 1 to 6 carbon atoms may be linear, branched, or cyclic, and examples thereof include a methyl group, an ethyl group, a n-propyl group, an isopropyl group, a cyclopropyl group, a n-butyl group, an isobutyl group, a sec-butyl group, a tert-butyl group, a cyclobutyl group, a n-pentyl group, a cyclopentyl group, a n-hexyl group, and a cyclohexyl group. Examples of the alkyl moiety of the alkoxy group having 1 to 6 carbon atoms, the acyloxy group having 2 to 7 carbon atoms, and the alkoxycarbonyl group having 2 to 7 carbon atoms include those the same as those disclosed as examples of the alkyl group, and examples of the alkyl moiety of the alkylsulfonyloxy group having 1 to 4 carbon atoms include those having 1 to 4 carbon atoms among the examples of the above-described alkyl group. The alkenyl group having 2 to 8 carbon atoms may be linear, branched, or cyclic, and examples thereof include a vinyl group, a 1-propenyl group, and a 2-propenyl group. Among them, a hydrogen atom, a fluorine atom, a chlorine atom, a hydroxy group, an amino group, an alkyl group having 1 to 3 carbon atoms, an alkoxy group having 1 to 3 carbon atoms, an acyloxy group having 2 to 4 carbon atoms, —NR^{ps1A}—C(═O)—R^{ps1B}, and —NR^{ps1A}—C(═O)—O—R^{ps1B} are preferable as R^{ps1}.

R^{ps1}, R^{ps2}, R^{ps3}, R^{ps4}, and R^{ps5} are each independently a monovalent hydrocarbon group having 1 to 20 carbon atoms and optionally containing a heteroatom. R^{ps1}, R^{ps2}, and R^{ps3} contain one or more fluorine atoms, and R^{ps4} and R^{ps5} contain one or more fluorine atoms. Any two of R^{ps1}, R^{ps2}, and R^{ps3} may be bonded to each other to form a ring together with the sulfur atom to which they are bonded. The monovalent hydrocarbon group may be linear, branched, or cyclic, and examples thereof include an alkyl group having 1 to 12 carbon atoms, an alkenyl group having 2 to 12 carbon atoms, an alkynyl group having 2 to 12 carbon atoms, an aryl group having 6 to 20 carbon atoms, and an aralkyl group having 7 to 12 carbon atoms. Some or all of the hydrogen atoms of these groups may be replaced by a hydroxy group, a carboxy group, a halogen atom, a cyano group, an amide group, a nitro group, a mercapto group, a sultone group, a sulfone group, or a sulfonium salt-containing group, and some of the carbon atoms of these groups may be replaced by an ether linkage, an ester linkage, a carbonyl group, a carbonate group, or a sulfonic acid ester linkage.

L^{ps1} is a single bond or a divalent organic group having 1 to 20 carbon atoms. Examples of the divalent linking group include groups formed by combining an ether linkage, a carbonyl group, an ester linkage, an amide linkage, a sultone ring, a lactam ring, a carbonate linkage, a carboxy group and a divalent hydrocarbon group, and the divalent hydrocarbon group may be substituted with a halogen atom, a hydroxy group or a carboxy group. Examples of the divalent hydrocarbon group include alkylene groups having 1 to 12 carbon atoms, cycloalkylene groups having 3 to 12 carbon atoms, or arylene groups having 6 to 10 carbon atoms, and examples thereof include the same groups as those disclosed as examples of X^{c2} in the formulas (2) and (3).

m^{ps} and n^{ps} are integers satisfying 0≤m^{ps}≤5, 0≤n^{ps}≤3, and 0≤m^{ps}+n^{ps}≤5, and integers satisfying 1≤m^{ps}≤3 and 0≤n^{ps}≤2 are preferable.

Examples of the anion of the acid diffusion controlling agent represented by the above formula (ps-1) or (ps-2) include, but are not limited to, those shown below. While all of those shown below are organic acid anion moieties having an iodine-substituted aromatic ring structure, organic acid anion moieties having no iodine-substituted aromatic ring structure that can be suitably employed include structures in which the iodine atoms in the formulas shown below are replaced by an atom or group other than an iodine atom such as a hydrogen atom or other substituent.

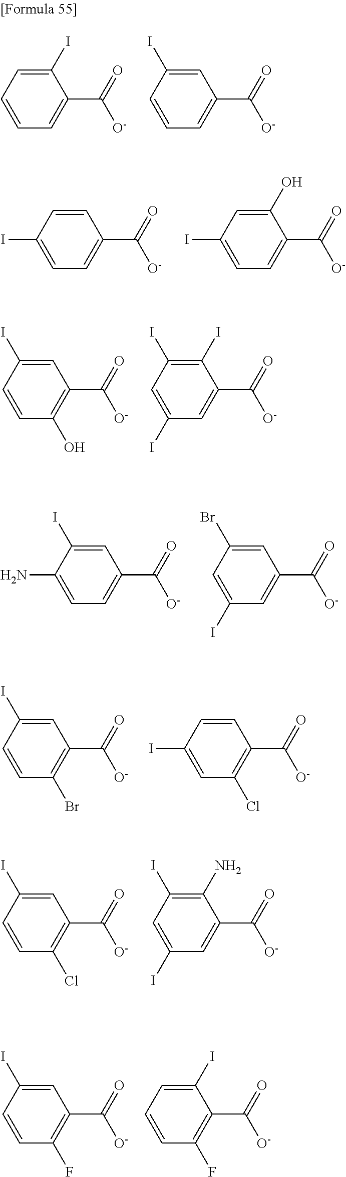

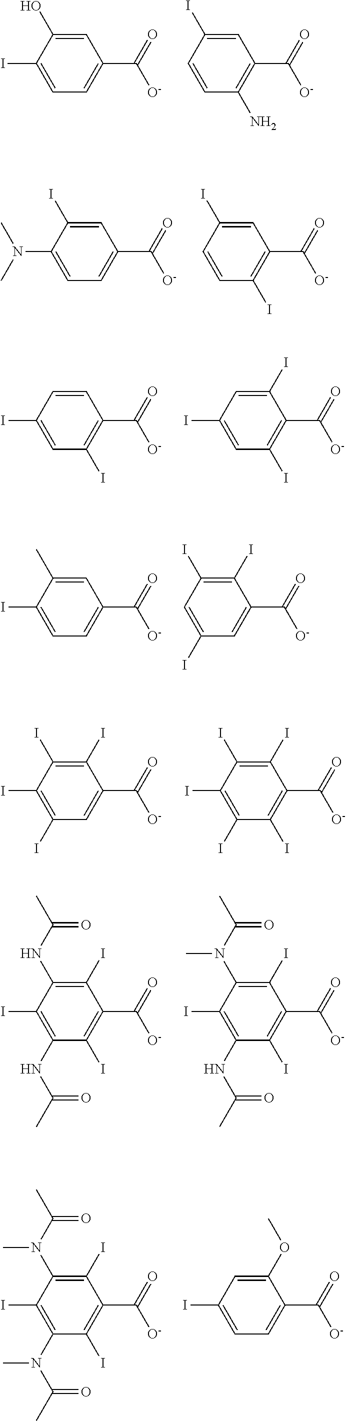

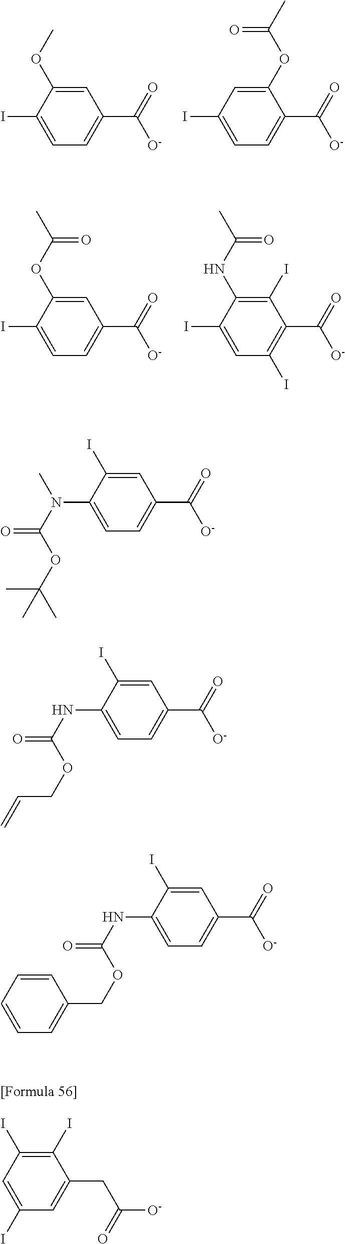

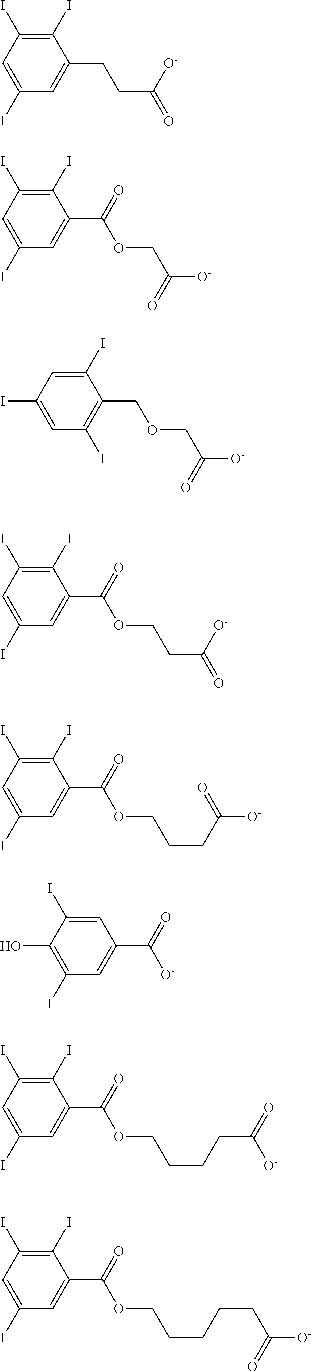

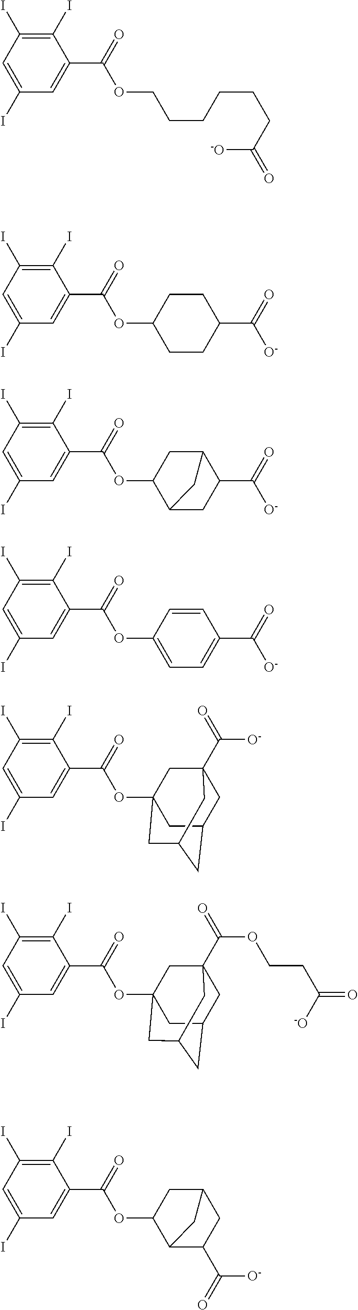

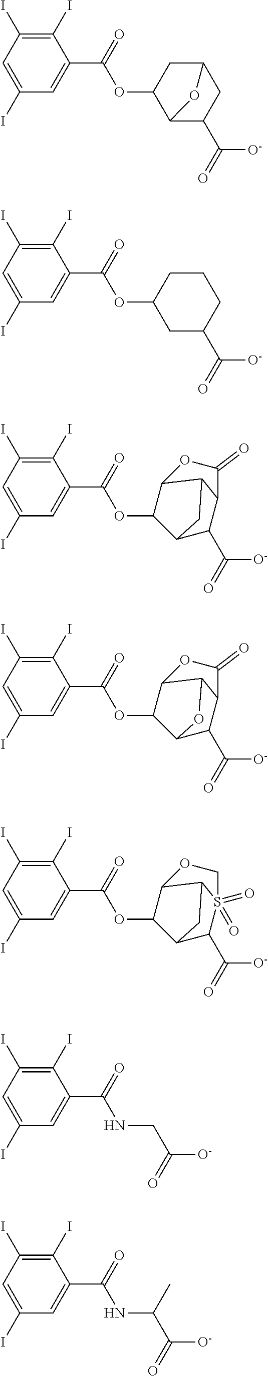

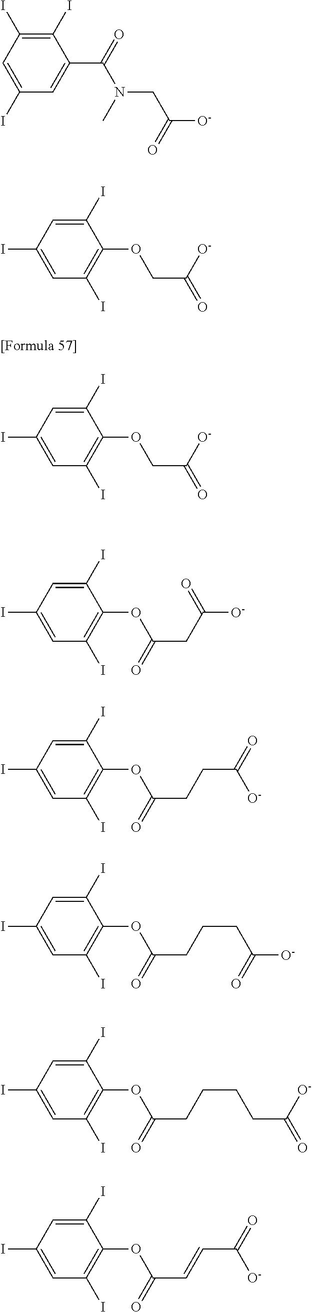

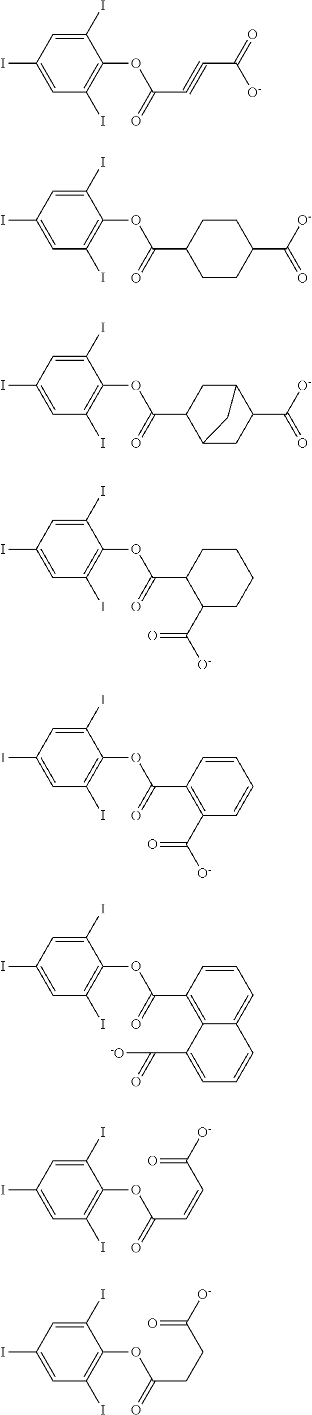

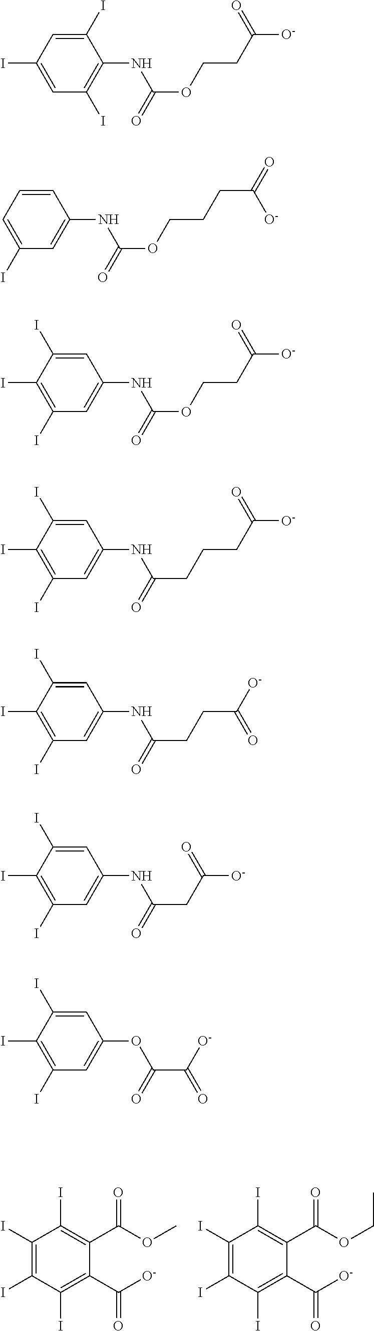

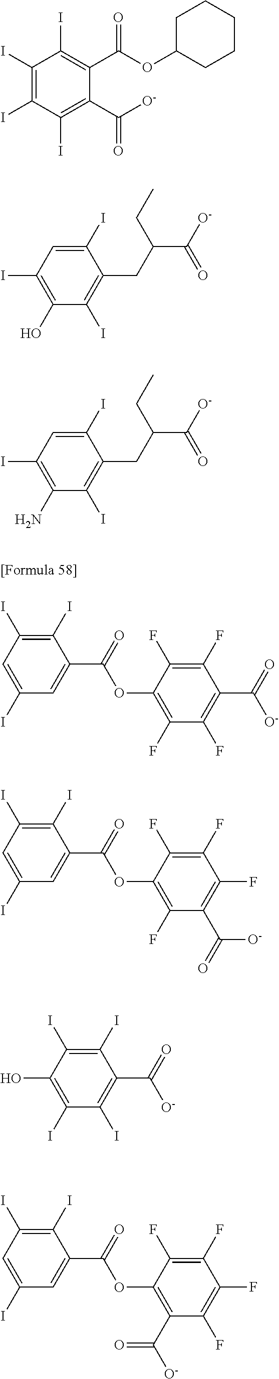

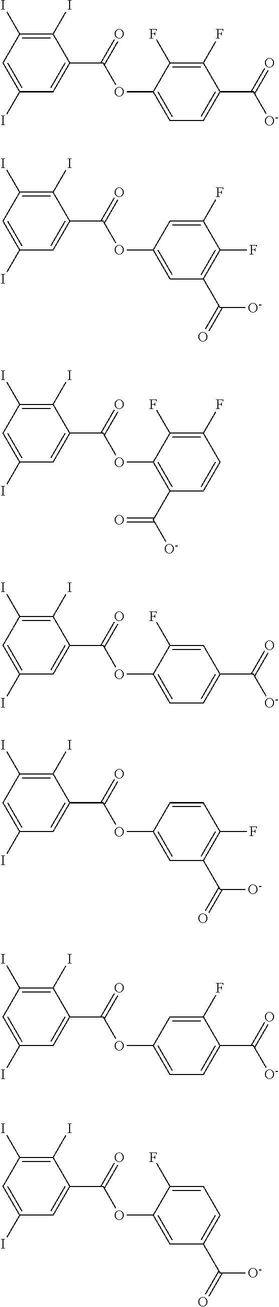

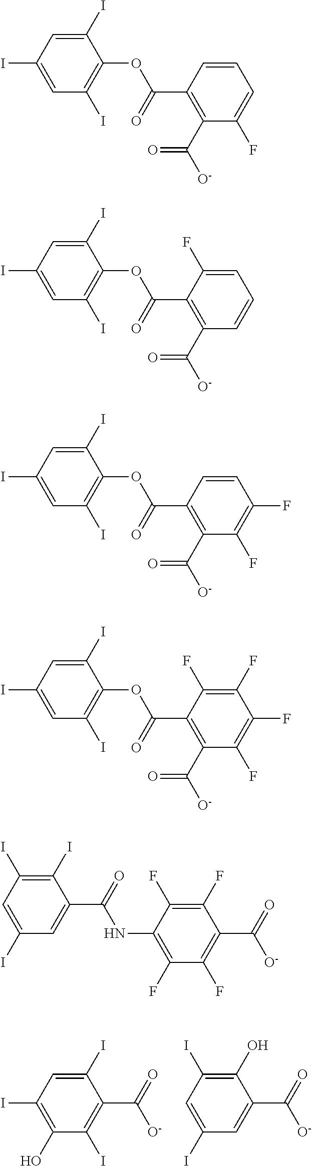

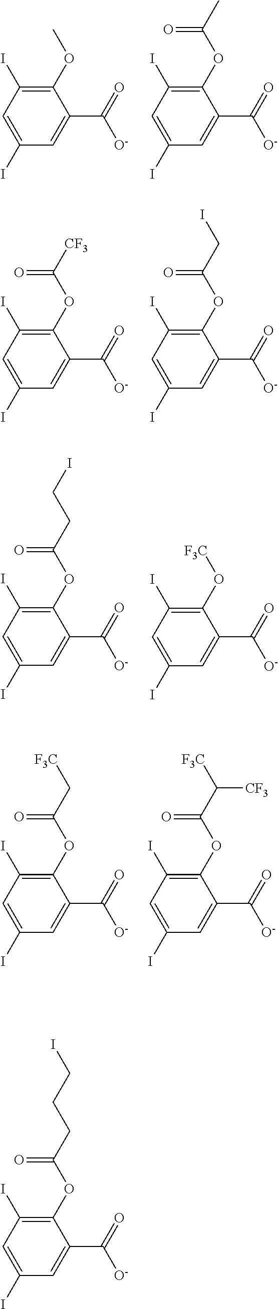

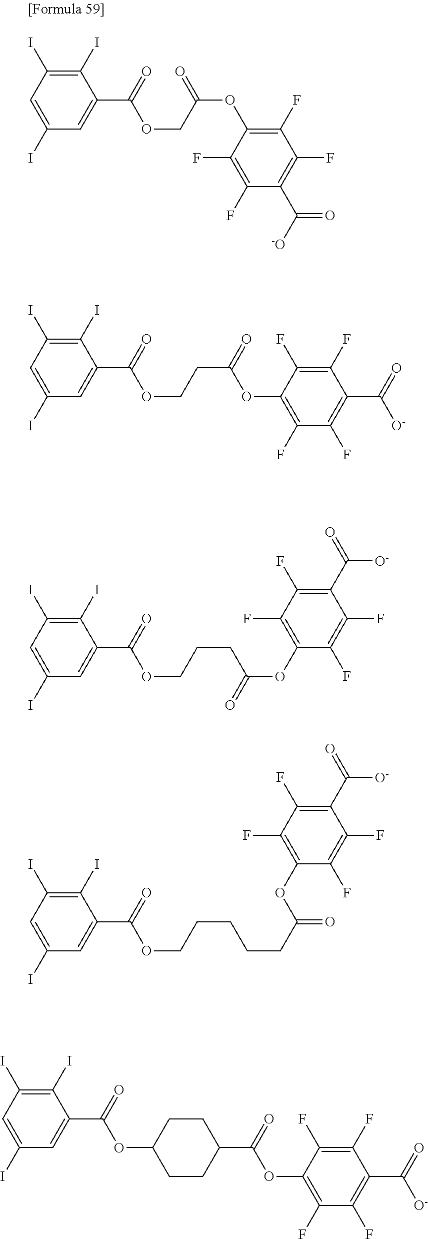

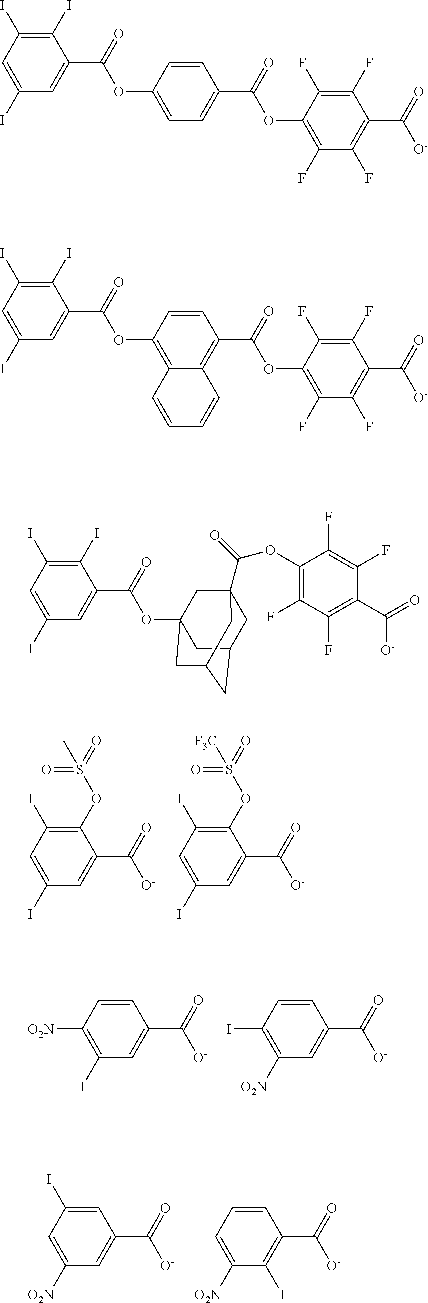

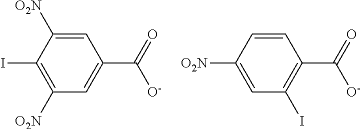 [SMILES: O=C([O-])c1cc([N+](=O)[O-])c(I)c([N+](=O)[O-])c1 ; O=C([O-])c1ccc([N+](=O)[O-])cc1I]

As the onium cation moieties in the acid diffusion controlling agents represented by the formulas (ps-1) and (ps-2), an onium cation moiety in a structural unit of a radiation-sensitive acid generating resin can be suitably employed.

The acid diffusion controlling agents represented by the above formulas (ps-1) and (ps-2) can also be synthesized by a known method, particularly by a salt exchange reaction.

These acid diffusion controlling agents may be used singly or two or more of them may be used in combination. The content of the acid diffusion controlling agent is preferably 10 parts by mass or more, more preferably 15 parts by mass or more, and still more preferably 20 parts by mass or more based on the content of the radiation-sensitive acid generator (when a radiation-sensitive acid generating resin is contained, the total amount with the content of the structural units in 100 parts by mass of the radiation-sensitive acid generating resin is taken as the basis). The content is preferably 100 mass % or less, more preferably 80 mass % or less, and still more preferably 60 mass % or less. This makes it possible to exhibit superior sensitivity or CDU performance when forming a resist pattern.

### <Compound>

The radiation-sensitive resin composition preferably contains, as a quencher, a compound having a structure in which an alkoxycarbonyl group is bonded to a nitrogen atom. Thanks to containing this compound, the diffusion length of a generated acid can be appropriately controlled and pattern-forming performance and CDU performance can be improved.

### <Solvent>

The radiation-sensitive resin composition according to the present embodiment contains a solvent. The solvent is not particularly limited as long as it is a solvent capable of dissolving or dispersing at least a base resin (the radiation-sensitive acid generating resin and at least one of the resins) and additives which are contained as desired.

Examples of the solvent include an alcohol-based solvent, an ether-based solvent, a ketone-based solvent, an amide-based solvent, an ester-based solvent, and a hydrocarbon-based solvent.

Examples of the alcohol-based solvent include:
a monoalcohol-based solvent having 1 to 18 carbon atoms, such as iso-propanol, 4-methyl-2-pentanol, 3-methoxybutanol, n-hexanol, 2-ethylhexanol, furfuryl alcohol, cyclohexanol, 3,3,5-trimethylcyclohexanol, and diacetone alcohol;
a polyhydric alcohol having 2 to 18 carbon atoms, such as ethylene glycol, 1,2-propylene glycol, 2-methyl-2,4-pentanediol, 2,5-hexanediol, diethylene glycol, dipropylene glycol, triethylene glycol, and tripropylene glycol; and
a partially etherized polyhydric alcohol-based solvent resulting from etherification of some of the hydroxy groups of the above-described polyhydric alcohol-based solvent.

Examples of the ether-based solvent include:
a dialkyl ether-based solvent, such as diethyl ether, dipropyl ether, and dibutyl ether;
a cyclic ether-based solvent, such as tetrahydrofuran and tetrahydropyran;
an aromatic ring-containing ether-based solvent, such as diphenyl ether and anisole (methyl phenyl ether); and
a polyhydric alcohol ether-based solvent resulting from etherification of hydroxy groups of the above-described polyhydric alcohol-based solvent.

Examples of the ketone-based solvent include a chain ketone-based solvent, such as acetone, butanone, and methyl-iso-butyl ketone;
a cyclic ketone-based solvent, such as cyclopentanone, cyclohexanone, and methylcyclohexanone; and 2,4-pentanedione, acetonylacetone, and acetophenone.

Examples of the amide-based solvent include a cyclic amide-based solvent, such as N,N′-dimethylimidazolidinone and N-methylpyrrolidone; and
a cyclic amide-based solvent, such as N-methylformamide, N,N-dimethylformamide, N,N-diethylformamide, acetamide, N-methylacetamide, N,N-dimethylacetamide, and N-methylpropionamide.

Examples of the ester-based solvent include:
a monocarboxylate ester-based solvent, such as n-butyl acetate and ethyl lactate;
a partially etherized polyhydric alcohol acetate-based solvent, such as diethylene glycol mono-n-butyl ether acetate, propylene glycol monomethyl ether acetate, and dipropylene glycol monomethyl ether acetate;
a lactone-based solvent, such as γ-butyrolactone and valerolactone;
a carbonate-based solvent, such as diethyl carbonate, ethylene carbonate, and propylene carbonate; and
a polyhydric carboxylic acid diester-based solvent, such as propylene glycol diacetate, methoxy triglycol acetate, diethyl oxalate, ethyl acetoacetate, ethyl lactate, and diethyl phthalate.

Examples of the hydrocarbon-based solvent include:
an aliphatic hydrocarbon-based solvent, such as n-hexane, cyclohexane, and methylcyclohexane; and
an aromatic hydrocarbon-based solvent, such as benzene, toluene, di-iso-propylbenzene, and n-amylnaphthalene.

Among them, ester-based solvents and ketone-based solvents are preferable, polyhydric alcohol partial ether acetate-based solvents, cyclic ketone-based solvents, and lactone-based solvents are more preferable, and propylene glycol monomethyl ether acetate, cyclohexanone, and γ-butyrolactone are still more preferable. The radiation-sensitive resin composition may contain one type or two or more types of solvent.

### <Other Optional Components>

The radiation-sensitive resin composition may contain other optional components in addition to the components described above. Examples of the other optional components include a crosslinking agent, a localization enhancing agent, a surfactant, an alicyclic backbone-containing compound, and a sensitizer. Such other optional components may be used singly or two or more types thereof may be used in combination.

In the case of using a radiation-sensitive acid generating resin containing a repeating unit A having an acid-dissociable group represented by the formula (1) and a repeating unit B containing an organic acid anion moiety and a sulfonium cation moiety containing an aromatic ring structure having a fluorine atom,
the radiation-sensitive resin composition preferably further contain at least one agent selected from the group consisting of:
a radiation-sensitive acid generator containing an organic acid anion moiety and an onium cation moiety; and
an acid diffusion controlling agent containing an organic acid anion moiety and an onium cation moiety and being to generate an acid having a pKa higher than that of an acid to be generated from the radiation-sensitive acid generator through irradiation with radiation.

In addition, in the case of using a radiation-sensitive resin composition comprising:
a radiation-sensitive acid generating resin containing a repeating unit A having an acid-dissociable group represented by the formula (1) and a repeating unit C having an organic acid anion moiety and an onium cation moiety,
an onium salt containing an organic acid anion moiety and a sulfonium cation containing an aromatic ring structure having a fluorine atom, and
a solvent,
the onium salt preferably comprises at least one agent selected from the group consisting of:
a radiation-sensitive acid generator containing an organic acid anion moiety and an onium cation moiety, and
an acid diffusion controlling agent containing an organic acid anion moiety and an onium cation moiety and being to generate an acid having a pKa higher than that of an acid to be generated from the radiation-sensitive acid generator through irradiation with radiation; and
at least one of the onium cation moiety constituting the radiation-sensitive acid generator and the onium cation moiety constituting the acid diffusion controlling agent is preferably a sulfonium cation containing an aromatic ring structure having a fluorine atom.

### <Method for Preparing Radiation-Sensitive Resin Composition>

The radiation-sensitive resin composition can be prepared, for example, by mixing a base resin (at least one of a radiation-sensitive acid generating resin and a resin) and a solvent, and if necessary, other optional component at a prescribed ratio. The radiation-sensitive resin composition is preferably filtered through, for example, a filter having a pore size of about 0.05 μm after mixing. The solid concentration of the radiation-sensitive resin composition is usually from 0.1% by mass to 50% by mass, preferably from 0.5% by mass to 30% by mass, and more preferably from 1% by mass to 20% by mass.

### <Pattern Forming Method>

The method for forming a resist pattern according to the present invention comprises:
step (1) of applying the radiation-sensitive resin composition directly or indirectly to a substrate to form a resist film (hereinafter also referred to as “resist film formation step”);
step (2) of exposing the resist film to light (hereinafter also referred to as “exposure step”); and
step (3) of developing the exposed resist film (hereinafter also referred to as “development step”).

In accordance with this method for forming a pattern, a high-quality resist pattern can be formed because of the use of the radiation-sensitive resin composition superior in sensitivity and CDU performance in an exposure step. Hereinbelow, each of the steps will be described.

### [Resist Film Forming Step]

In this step (step (1)), a resist film is formed from the radiation-sensitive resin composition. Examples of the substrate on which the resist film is formed include conventionally known substrates such as a silicon wafer, silicon dioxide, and a wafer coated with aluminum. An organic or inorganic antireflective film disclosed in, for example, JP-B-6-12452 or JP-A-59-93448 may be formed on the substrate. Examples of a method for applying the composition include spin coating, cast coating, and roll coating. After the application, prebaking (PB) may be performed to volatilize the solvent in the coating film, as necessary. The PB temperature is usually 60° C. to 140° C., and preferably 80° C. to 120° C. The PB time is usually 5 seconds to 600 seconds, and preferably 10 seconds to 300 seconds. The thickness of the resist film to be formed is preferably 10 nm to 1,000 nm, and more preferably 10 nm to 500 nm.

In the case of performing immersion exposure, regardless of the presence or absence of a water repellent polymer additive such as the high fluorine-containing resin in the radiation-sensitive resin composition, a protective film for immersion insoluble in an immersion liquid may be provided on the formed resist film for the purpose of avoiding direct contact between the immersion liquid and the resist film. As the protective film for immersion, either a solvent-removable protective film that is to be removed by a solvent before the development step (see, for example, JP-A-2006-227632) or a developer-removable protective film that is to be removed simultaneously with the development in the development step (see, for example, WO 2005 069076 and WO 2006 035790) may be used. However, from the viewpoint of throughput, it is preferable to use a developer-removable protective film for immersion.

When the subsequent exposure step is performed using radiation having a wavelength of 50 nm or less, it is preferable to use a resin having the structural units b1, b2, the structural unit c, and, as necessary, a structural unit d as the base resin in the composition.

### [Exposure Step]

In this step (the step (2)), the resist film formed in the resist film forming step, namely the step (1), is irradiated with radiation through a photomask (as the case may be, through an immersion medium such as water) to be exposed. Examples of the radiation to be used for the exposure include an electromagnetic wave including visible ray, ultraviolet ray, far ultraviolet ray, extreme ultraviolet ray (EUV), X ray, and γ ray; an electron beam; and a charged particle radiation such as a ray. Among them, far ultraviolet ray, electron beam, and EUV are preferable, ArF excimer laser light (wavelength: 193 nm), KrF excimer laser light (wavelength: 248 nm), electron beam, and EUV are more preferable, and an electron beam and EUV having a wavelength of 50 nm or less, which are positioned as next-generation exposure technology, are still more preferable.

When the exposure is performed by immersion exposure, examples of the immersion liquid to be used include water and a fluorine-based inert liquid. The immersion liquid is preferably a liquid that is transparent to an exposure wavelength and has a temperature coefficient of refractive index as small as possible to minimize the distortion of an optical image projected onto the film. Particularly, when an exposure light source is ArF excimer laser light (wavelength: 193 nm), water is preferably used from the viewpoint of availability and ease of handling in addition to the above-described viewpoints. When water is used, an additive that reduces the surface tension of water and increases the surface activity may be added in a small proportion. This additive is preferably one that does not dissolve the resist film on a wafer and has negligible influence on an optical coating at an under surface of a lens. The water to be used is preferably distilled water.

After the exposure, post exposure baking (PEB) is preferably carried out to promote the dissociation of the acid-dissociable group of the resin or the like due to the acid generated from the radiation-sensitive acid generator through the exposure in the exposed area of the resist film. As a result of the PEB, there is produced a difference in solubility in the developer between the exposed area and the unexposed area. The PEB temperature is usually 50° C. to 180° C., and preferably 80° C. to 130° C. The PEB time is usually 5 seconds to 600 seconds, and preferably 10 seconds to 300 seconds.

### [Development Step]

In this step (the step (3)), the resist film exposed in the exposure step, namely the step (2), is developed. Thus, a prescribed resist pattern can be formed. In a common procedure, after the development, the film is washed with a rinsing liquid such as water or alcohol and dried.

Examples of the developer to be used for the development include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, and 1,5-diazabicyclo-[4.3.0]-5-nonene. Among them, an aqueous TMAH solution is preferable, and a 2.38% by mass aqueous TMAH solution is more preferable.

In the case of organic solvent development, examples of the solvent include organic solvents such as hydrocarbon-based solvents, ether-based solvents, ester-based solvents, ketone-based solvents, and alcohol-based solvents, and solvents containing an organic solvent. Examples of the organic solvent include one type or two or more types of solvent among the solvents listed as the solvent for the radiation-sensitive resin composition. Among them, ester-based solvents and ketone-based solvents are preferable. As the ester-based solvents, acetate-based solvents are preferable, and n-butyl acetate and amyl acetate are more preferable. As the ketone-based solvents, chain ketones are preferable, and 2-heptanone is more preferable. The content of the organic solvent in the developer is preferably 80% by mass or more, more preferably 90% by mass or more, still more preferably 95% by mass, and particularly preferably 99% by mass. Examples of the components other than the organic solvent in the developer include water and silicon oil.

Examples of a development method include a method in which a substrate is immersed in a bath filled with a developer for a certain period of time (dipping method), a method in which a developer is allowed to be present on a surface of a substrate due to surface tension and to stand for a certain period of time (puddle method), a method in which a developer is sprayed onto a surface of a substrate (spray method), and a method in which a developer is discharged onto a substrate that is rotated at a constant speed while a developer discharge nozzle is scanned at a constant speed (dynamic dispensing method).

### EXAMPLES

Hereinafter, the present invention will specifically be described with reference to synthesis examples, examples, and comparative examples, but is not limited to the following examples. Methods for measuring various physical property values will be described below.

### [Mw and Mn]

The Mw and the Mn of polymers were measured by gel permeation chromatography (GPC) using GPC columns manufactured by Tosoh Corporation (“G2000HXL” x 2, “G3000HXL” x 1, “G4000HXL” x 1) under the following conditions.
Eluant: tetrahydrofuran (manufactured by Wako Pure Chemical Industries, Ltd.)
Flow rate: 1.0 mL/min
Sample concentration: 1.0% by mass
Amount of sample injected: 100 μL
Column temperature: 40° C.
Detector: differential refractometer
Standard substance: monodisperse polystyrene

The structures of the sulfonium salt or iodonium salt acid generators PAG1 to PAG4 used for the resist materials of Examples are given below.

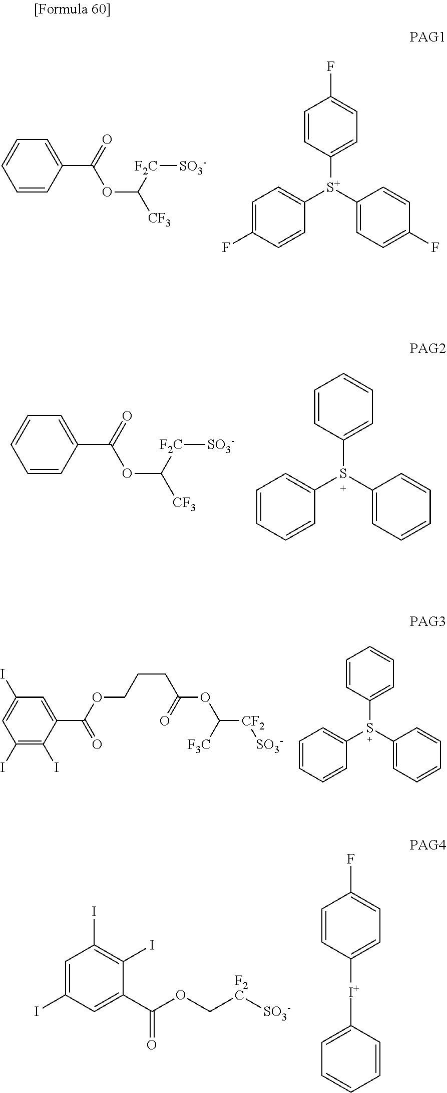

### [Synthesis Example]Synthesis of Base Polymers (P-1 to P-12)

The respective monomers were combined and subjected to a copolymerization reaction in a tetrahydrofuran (THF) solvent, and the reaction products were crystallized in methanol, and washed repeatedly with hexane, then isolated, and dried. Thus, base polymers (P-1 to P-12) having the compositions shown below were obtained. The composition of the obtained base polymers was confirmed by 1H-NMR, and the Mw and the dispersion degree (Mw/Mn) were confirmed by GPC (solvent: THF, standard: polystyrene).
P-1: Mw=9,200, Mw/Mn=1.8
P-2: Mw=9,100, Mw/Mn=1.8
P-3: Mw=9,300, Mw/Mn=1.7
P-4: Mw=9,200, Mw/Mn=1.8
P-5: Mw=8,900, Mw/Mn=1.8
P-6: Mw=9,200, Mw/Mn=1.8
P-7: Mw=9,200, Mw/Mn=1.8
P-8: Mw=9,200, Mw/Mn=1.8
P-9: Mw=9,200, Mw/Mn=1.8
P-10: Mw=9,200, Mw/Mn=1.8
P-11: Mw=9,700, Mw/Mn=1.7
P-12: Mw=9,400, Mw/Mn=1.8

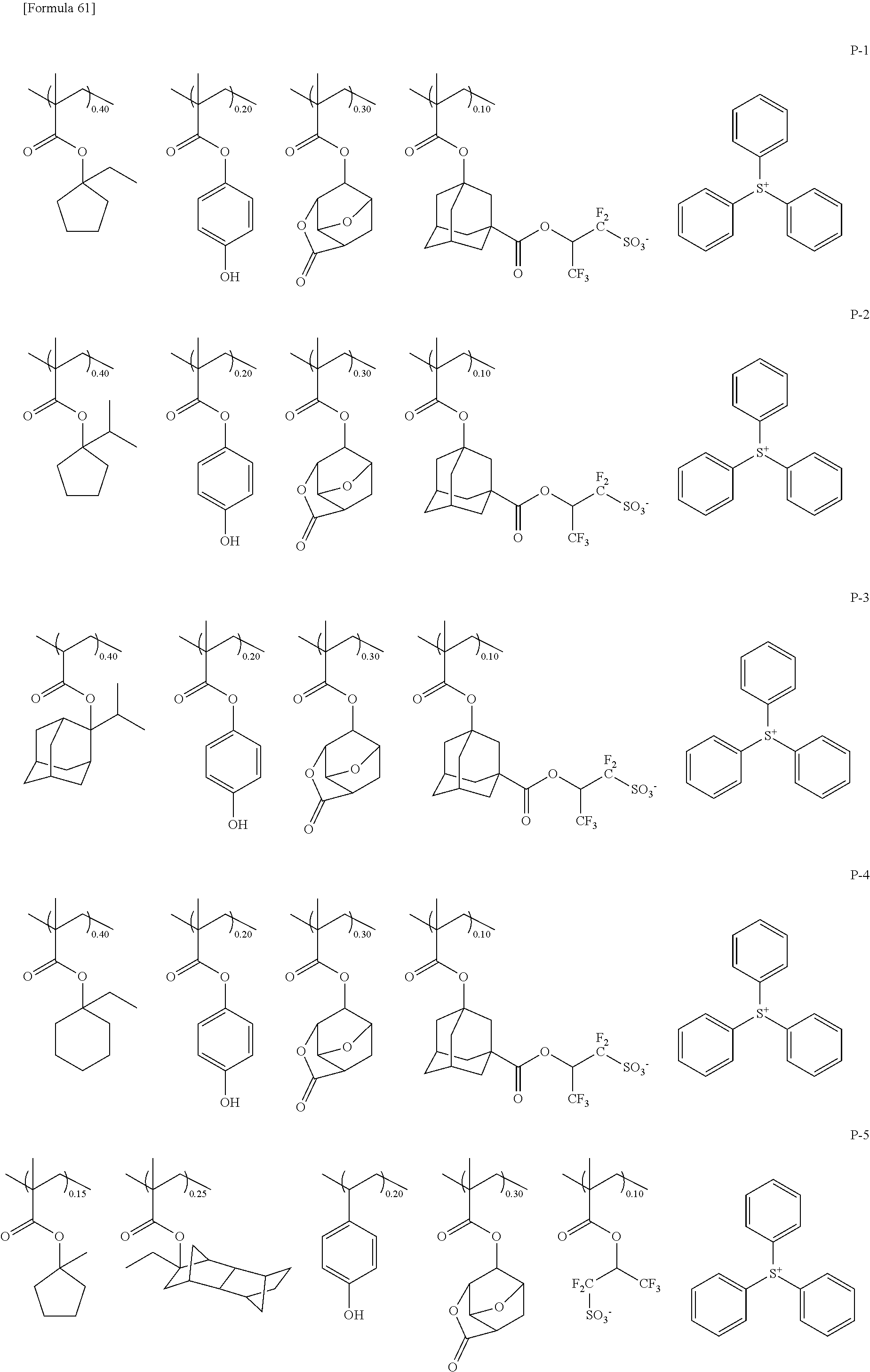

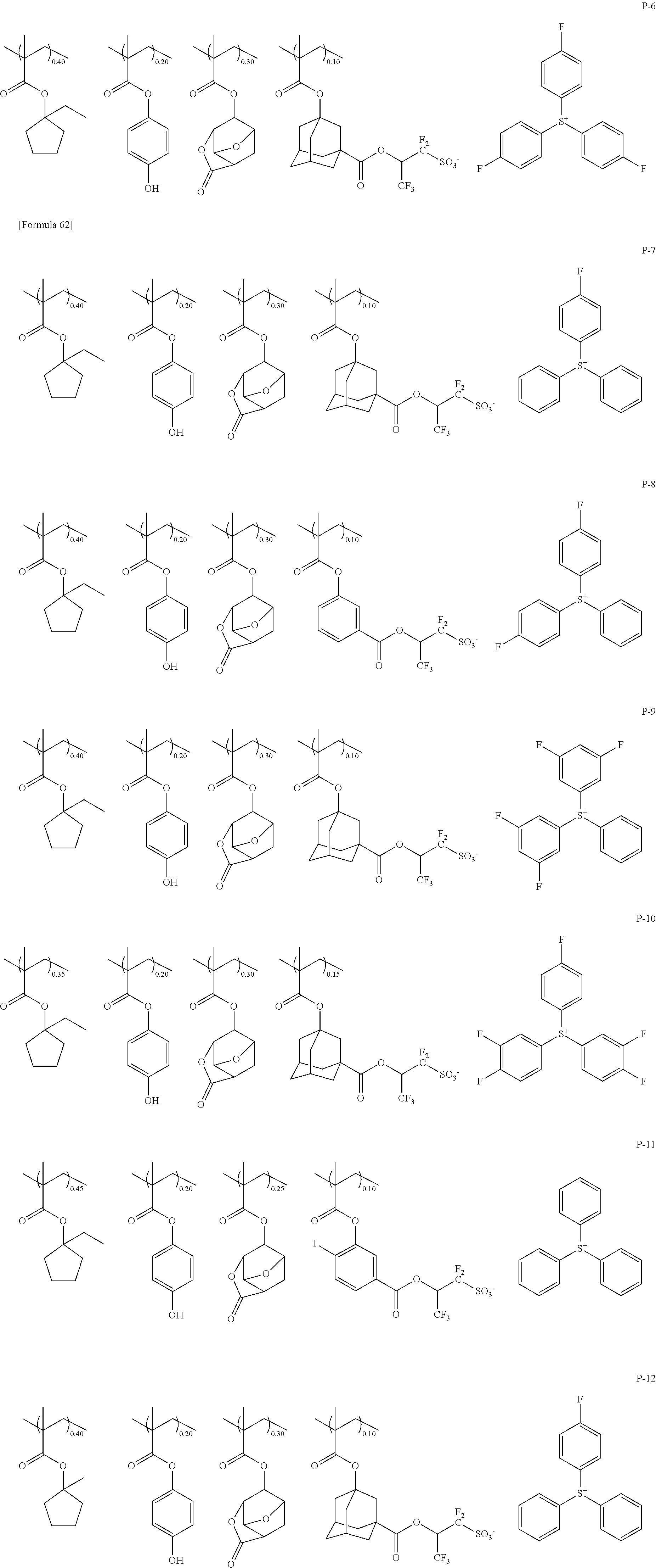

### Examples and Comparative Examples

A resist material was prepared by filtering a solution obtained by dissolving components in the composition given in Table 2 in a solvent obtained by dissolving 100 ppm of FC-4430 manufactured by 3M as a surfactant through a 0.2 μm-sized filter.

In Table 1, the components are as follows.
Organic solvent: PGMEA (propylene glycol monomethyl ether acetate)
GBL (γ-butyrolactone)
CHN (cyclohexanone)
PGME (propylene glycol monomethyl ether)
DAA (diacetone alcohol)

Acid diffusion controlling agents 1 to 3 (see the following structural formulas)

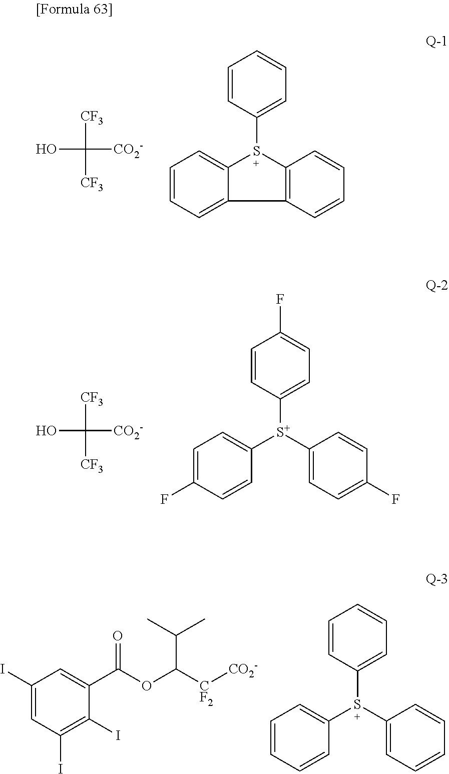

High fluorine-containing resin F-1: Mw=8,900, Mw/Mn=2.0

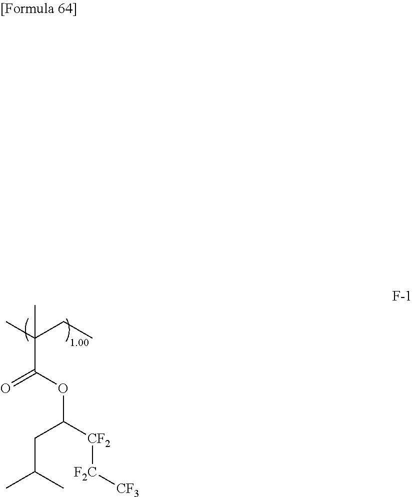

### [EUV Exposure Evaluation]

### Examples 1 to 15 and Comparative Examples 1 to 3

Each of the radiation-sensitive resin compositions shown in Table 1 was spin-coated on a Si substrate formed in a film thickness of 20 nm from a silicon-containing spin-on hard mask SHB-A940 (silicon content: 43 mass %) manufactured by Shin-Etsu Chemical Co., Ltd. Prebaking was carried out at 105° C. for 60 seconds using a hot plate to prepare a resist film having a thickness of 60 nm. This was exposed to light using an EUV scanner NXE3300 (NA 0.33, σ 0.9/0.6, quadrupole illumination, hole pattern mask with a pitch of 46 nm on wafer and a bias of +20%) manufactured by ASML. PEB was carried out on a hot plate at 100° C. for 60 seconds. Development was carried out with a 2.38 mass % aqueous TMAH solution for 30 seconds, affording a hole pattern having a dimension of 23 nm. The exposure dose applied when a pattern was formed with a hole dimension of 23 nm was measured and taken as sensitivity. In addition, dimensions of 50 holes were measured using a critical dimension-SEM (CG5000) manufactured by Hitachi High-Technologies Corporation to determine CDU (dimensional variation 3σ). The results are shown in Table 1.

TABLE 1

| | Base | | Acid diffusion | | | | |
|---|---|---|---|---|---|---|---|
| | resin | PAG | controlling | | Additive | | |
| | (parts by | (parts by | agent | Solvent | (parts by | Sensitivity | CDU |
| | mass) | mass) | (parts by mass) | (parts by mass) | mass) | [mJ/cm²] | [nm] |
| Example 1 | P-1 | PAG1 | Q-1 | PGMEA/DAA | F-1 | 14 | 2.5 |
| | (100) | (8.0) | (4.0) | (2,000/500) | (3.0) | | |
| Example 2 | P-2 | PAG1 | Q-1 | PGMEA/DAA | F-1 | 14 | 2.5 |
| | (100) | (8.0) | (4.0) | (2,000/500) | (3.0) | | |
| Example 3 | P-3 | PAG1 | Q-1 | PGMEA/DAA | F-1 | 13 | 2.5 |
| | (100) | (8.0) | (4.0) | (2,000/500) | (3.0) | | |
| Example 4 | P-4 | PAG1 | Q-1 | PGMEA/DAA | F-1 | 14 | 2.5 |
| | (100) | (8.0) | (4.0) | (2,000/500) | (3.0) | | |
| Example 5 | P-5 | PAG1 | Q-1 | PGMEA/DAA | F-1 | 13 | 2.5 |
| | (100) | (8.0) | (4.0) | (2,000/500) | (3.0) | | |
| Example 6 | P-1 | PAG2 | Q-2 | PGMEA/DAA | F-1 | 14 | 2.5 |
| | (100) | (8.0) | (4.0) | (2,000/500) | (3.0) | | |
| Example 7 | P-6 | PAG2 | Q-1 | PGMEA/DAA | F-1 | 14 | 2.5 |
| | (100) | (8.0) | (4.0) | (2,000/500) | (3.0) | | |
| Example 8 | P-7 | PAG2 | Q-1 | PGMEA/DAA | F-1 | 15 | 2.5 |
| | (100) | (8.0) | (4.0) | (2,000/500) | (3.0) | | |
| Example 9 | P-8 | PAG3 | Q-1 | PGMEA/DAA | F-1 | 15 | 2.3 |
| | (100) | (8.0) | (4.0) | (2,000/500) | (3.0) | | |
| Example 10 | P-9 | PAG2 | Q-1 | PGMEA/DAA | F-1 | 14 | 2.5 |
| | (100) | (8.0) | (4.0) | (2,000/500) | (3.0) | | |
| Example 11 | P-10 | PAG2 | Q-1 | PGMEA/DAA | F-1 | 13 | 2.5 |
| | (100) | (8.0) | (4.0) | (2,000/500) | (3.0) | | |
| Example 12 | P-6 | PAG1 | Q-3 | PGMEA/CHN/PGME | F-1 | 14 | 2.3 |
| | (100) | (7.0) | (3.5) | (400/2,000/100) | (3.0) | | |
| Example 13 | P-11 | PAG1 | Q-2 | PGMEA/CHN/PGME | F-1 | 13 | 2.3 |
| | (100) | (7.0) | (3.5) | (400/2,000/100) | (3.0) | | |
| Example 14 | P-6 | PAG2 | Q-2 | PGMEA/CHN/PGME | F-1 | 14 | 2.5 |
| | (100) | (7.0) | (3.5) | (400/2,000/100) | (3.0) | | |
| Example 15 | P-6 | PAG1 | Q-2 | PGMEA/CHN/PGME | F-1 | 13 | 2.5 |
| | (100) | (6.0) | (3.5) | (400/2,000/100) | (3.0) | | |
| Comparative | P-12 | PAG2 | Q-1 | PGMEA/GBL | F-1 | 16 | 2.5 |
| Example 1 | (100) | (8.0) | (4.0) | (2,200/300) | (3.0) | | |
| Comparative | P-1 | PAG4 | Q-1 | PGMEA/GBL | F-1 | 16 | 2.3 |
| Example 2 | (100) | (8.0) | (4.0) | (2,200/300) | (3.0) | | |
| Comparative | P-1 | PAG2 | Q-1 | PGMEA/GBL/PGME | F-1 | 16 | 2.5 |
| Example 3 | (100) | (8.0) | (4.0) | (2,200/200/100) | (3.0) | | |

The evaluation conducted for the resist patterns formed through the EUV exposure revealed that the radiation-sensitive resin compositions of Examples had good sensitivity and CDU performance.

### INDUSTRIAL APPLICABILITY

According to the radiation-sensitive resin composition and the method for forming a resist pattern described above, a resist pattern having good sensitivity to exposure light and excellent CDU performance can be formed. Therefore, these can be suitably used for a machining process and the like of a semiconductor device in which micronization is expected to further progress in the future.

1: A radiation-sensitive resin composition comprising:
a radiation-sensitive acid generating resin comprising: a repeating unit A comprising an acid-dissociable group represented by formula (1); and a repeating unit B comprising an organic acid anion moiety and a sulfonium cation moiety which comprises an aromatic ring structure having a fluorine atom; and
a solvent, 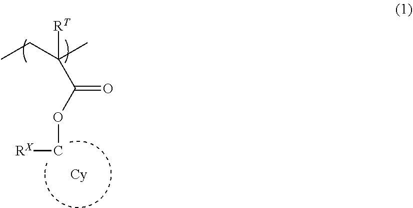
wherein, in the formula (1):
R^{T} is a hydrogen atom, a fluorine atom, a methyl group, or a trifluoromethyl group;
R^{X} is a monovalent hydrocarbon group having 2 to 20 carbon atoms; and
Cy represents an alicyclic structure having 3 to 20 ring members and formed together with a carbon atom to which Cy is bonded.

2: The radiation-sensitive resin composition according to claim 1, further comprising at least one selected from the group consisting of:
a radiation-sensitive acid generator comprising an organic acid anion moiety and an onium cation moiety; and
an acid diffusion controlling agent comprising an organic acid anion moiety and an onium cation moiety and being to generate an acid having a pKa higher than a pKa of an acid to be generated from the radiation-sensitive acid generator through irradiation with radiation.

3: The radiation-sensitive resin composition according to claim 1, wherein the repeating unit B is a repeating unit derived from a monomer represented by formula (2) or from a monomer represented by formula (3), 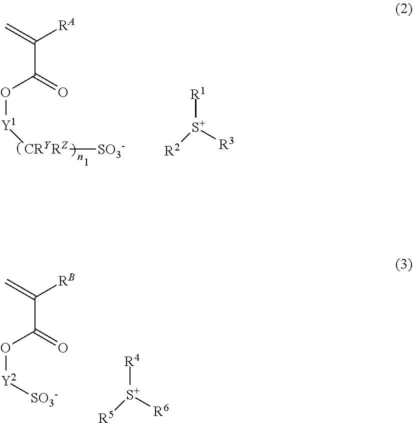
wherein, in the formulas (2) and (3),
R^{A} and R^{B} are each a hydrogen atom, a fluorine atom, a methyl group, or a trifluoromethyl group; and
R^{Y} and R^{Z} are each independently a hydrogen atom, a fluorine atom, or a fluorinated hydrocarbon group, at least one of R^{Y} and R^{Z} is a fluorine atom or a fluorinated hydrocarbon group, and when there are a plurality of R^{Y}s, the plurality of R^{Y}s are the same or different, and when there are a plurality of R^{Z}s, the plurality of R^{Z}s are the same or different;
n₁ is an integer of 1 to 20;
R¹ to R³ are each independently a monovalent hydrocarbon group, and at least one of R¹ to R³ is an aromatic ring having a fluorine atom;
R⁴ to R⁶ are each independently a monovalent hydrocarbon group, and at least one of R⁴ to R⁶ is an aromatic ring having a fluorine atom;
Y¹ is a single bond or —Y¹¹—C(═O)—O—, wherein Y¹¹ is a divalent hydrocarbon group having 1 to 20 carbon atoms and optionally comprising a heteroatom;
Y² is a single bond, a methylene group, an ethylene group, a phenylene group, a fluorinated phenylene group, —O—Y²¹—, —C(═O)—O—Y²¹—, or —C(═O)—NH—Y²¹—, wherein Y²¹ is an alkanediyl group having 1 to 6 carbon atoms, an alkenediyl group having 2 to 6 carbon atoms, or a phenylene group, and optionally comprises a carbonyl group, an ester linkage, an ether linkage, or a hydroxy group, and the alkanediyl group having 1 to 6 carbon atoms, the alkenediyl group having 2 to 6 carbon atoms, and the phenylene group are each optionally substituted with a fluorine atom.

4: A radiation-sensitive resin composition comprising:
a radiation-sensitive acid generating resin comprising: a repeating unit A which comprises an acid-dissociable group represented by formula (1); and a repeating unit C comprising an organic acid anion moiety and an onium cation moiety;
an onium salt comprising: an organic acid anion moiety; and a sulfonium cation comprising an aromatic ring structure having a fluorine atom; and
a solvent, 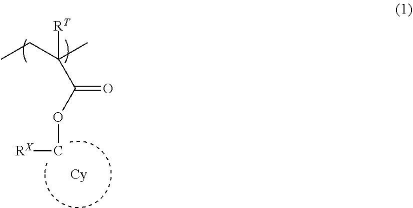
wherein, in the formula (1),
R^{T} is a hydrogen atom, a fluorine atom, a methyl group, or a trifluoromethyl group;
R^{X} is a monovalent hydrocarbon group having 1 to 20 carbon atoms; and
Cy represents a polycyclic alicyclic structure having 4 to 20 ring members formed together with the carbon atom to which Cy is bonded.

5: The radiation-sensitive resin composition according to claim 4, wherein the onium salt comprises at least one selected from the group consisting of:
a radiation-sensitive acid generator comprising an organic acid anion moiety and an onium cation moiety; and
an acid diffusion controlling agent comprising an organic acid anion moiety and an onium cation moiety and being to generate an acid having a pKa higher than a pKa of an acid to be generated from the radiation-sensitive acid generator through irradiation with radiation, and
at least one of the onium cation moiety constituting the radiation-sensitive acid generator and the onium cation moiety constituting the acid diffusion controlling agent is a sulfonium cation comprising an aromatic ring structure having a fluorine atom.

6: The radiation-sensitive resin composition according to claim 1, wherein the radiation-sensitive acid generating resin further comprises a structural unit D comprising a phenolic hydroxy group.

7: The radiation-sensitive resin composition according to claim 1, wherein the repeating unit C is a repeating unit derived from a monomer represented by formula (4) or from a monomer represented by formula (5): 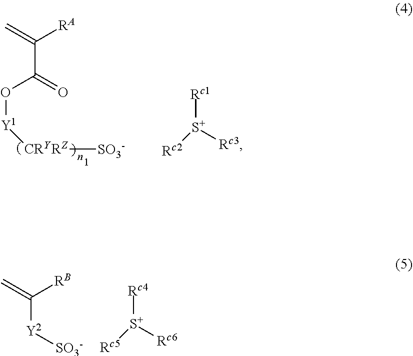
wherein, in the formulas (4) and (5):
R^{A} and R^{B} are each a hydrogen atom, a fluorine atom, a methyl group, or a trifluoromethyl group;
R^{Y} and R^{Z} are each independently a fluorine atom or a fluorinated hydrocarbon group, and at least one of R^{Y} and R^{Z} is a fluorine atom or a fluorinated hydrocarbon group, and when there are a plurality of R^{Y}s, the plurality of R^{Y}s are the same or different, and when there are a plurality of R^{Z}s, the plurality of R^{Z}s are the same or different;
n₁ is an integer of 1 to 20;
R^{c1} to R^{c3} are each independently a monovalent hydrocarbon group;
R^{c4} to R^{c6} are each independently a monovalent hydrocarbon group;
Y¹ is a single bond or —Y¹¹—C(═O)—O—, wherein Y¹¹ is a divalent hydrocarbon group having 1 to 20 carbon atoms and optionally comprising a heteroatom; and
Y² is a single bond, a methylene group, an ethylene group, a phenylene group, a fluorinated phenylene group, —O—Y²¹—, —C(═O)—O—Y²¹—, or —C(═O)—NH—Y²¹—, wherein Y²¹ is an alkanediyl group having 1 to 6 carbon atoms, an alkenediyl group having 2 to 6 carbon atoms, or a phenylene group, and optionally comprises a carbonyl group, an ester linkage, an ether linkage, or a hydroxy group.

8: The radiation-sensitive resin composition according to claim 1, wherein the radiation-sensitive acid generating resin further comprises a structural unit represented by formula (6): 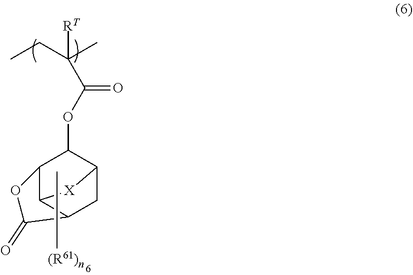
wherein, in the formula (6):
X is a methanediyl group unsubstituted or substituted with R⁶¹, or —O—;
R^{T} is a hydrogen atom, a fluorine atom, a methyl group, or a trifluoromethyl group;
R⁶¹ is a monovalent hydrocarbon group having 1 to 20 carbon atoms, wherein when there are a plurality of R⁶¹s, the plurality of R⁶¹s are the same or different; and
n₆ is an integer of 0 to 3.

9: The radiation-sensitive resin composition according to claim 1, wherein the organic acid anion moiety of at least one member selected from the group consisting of the radiation-sensitive acid generating resin, the radiation-sensitive acid generator, and the acid diffusion controlling agent comprises an iodine-substituted aromatic ring structure.

10: A method for forming a pattern, the method comprising:
directly or indirectly applying the radiation-sensitive resin composition according to claim 1 to a substrate to form a resist film;
exposing the resist film to light; and
developing the exposed resist film with a developer.

11: The method for forming a resist pattern according to claim 10, wherein the exposure is performed using extreme ultraviolet ray or electron beam.
